(19) United States
(12) Patent Application Publication
(10) Pub. No.: US 2022/0409684 A1
(43) Pub. Date: Dec. 29, 2022

(54) CODON OPTIMIZED NEW GENERATION REGULATABLE FUSOGENIC ONCOLYTIC HERPES SIMPLEX VIRUS TYPE 1 VIRUS AND METHODS OF USE

(71) Applicant: THE BRIGHAM AND WOMEN'S HOSPITAL, INC., Boston, MA (US)

(72) Inventor: Feng YAO, Southborough, MA (US)

(73) Assignee: THE BRIGHAM AND WOMEN'S HOSPITAL, INC., Boston, MA (US)

(21) Appl. No.: 17/776,828

(22) PCT Filed: Nov. 13, 2020
(86) PCT No.: PCT/US2020/060372

§ 371 Date: May 13, 2022

Related U.S. Application Data
(60) Provisional application No. 62/936,776, filed on Nov. 18, 2019.

(51) Int. Cl. A61K 35/763; C12N 7/00; A61K 31/65; C07K 14/495; A61K 35/17; A61K 45/06

(52) U.S. Cl. CPC A61K 35/763; C12N 7/00; A61K 31/65; C07K 14/495; A61K 35/17; A61K 45/06; C12N 2710/16621; C12N 2830/006; C12N 2710/16632; C07K 2319/03; C07K 2319/02

(57) ABSTRACT

Malignant tumors that are resistant to conventional therapies represent significant therapeutic challenges. An embodiment of the present invention provides a codon optimized new generation regulatable fusogenic oncolytic herpes simplex virus-1 that is more effective at selective killing target cells, such as tumor cells. In various embodiments presented herein, the oncolytic virus described herein is suitable for treatment of solid tumors, as well as other cancers.

### CROSS-REFERENCE TO RELATED APPLICATIONS

This application claims benefit under 35 U.S.C. § 119(e) of U.S. Provisional Application No. 62/936,776 filed Nov. 18, 2019, the contents of which is incorporated herein by reference in its entirety.

### FIELD OF INVENTION

The present invention is directed compositions and methods of treating cancer using a codon optimized regulatable fusogenic oncolytic herpes simplex virus 1 (HSV-1) virus.

### BACKGROUND

Oncolytic viral therapy entails harnessing the ability of a virus to reproduce in and lyse human cells and directing this viral replication-dependent lysis preferentially toward cancerous cells. Advances in cancer biology, together with a detailed understanding of the roles of host factors and virus-encoded gene products in controlling virus production in infected cells, have facilitated the use of some viruses as potential therapeutic agents against cancer (Aghi and Martuza, 2005; Parato et al., 2005). Herpes simplex virus (HSV) possesses several unique properties as an oncolytic agent (Aghi and Martuza, 2005). It can infect a broad range of cell types, leading to the replication of new virus and cell death. HSV has a short replication cycle (9 to 18 h) and encodes many non-essential genes that, when deleted, greatly restrict the ability of the virus to replicate in non-dividing normal cells. Because of its large genome, multiple therapeutic genes can be packaged into the genome of oncolytic recombinants.

The use of a replication-conditional strain of HSV-1 as an oncolytic agent was first reported for the treatment of malignant gliomas (Martuza et al., 1991). Since then, various efforts have been made in an attempt to broaden their therapeutic efficacy and increase the replication specificity of the virus in tumor cells. Not surprisingly, however, deletion of genes that impair viral replication in normal cells also leads to a marked decrease in the oncolytic activity of the virus for the targeted tumor cells (Advani et al., 1998; Chung et al., 1999). Currently, no oncolytic viruses that are able to kill only tumor cells while leaving normal cells intact are available. Consequently, the therapeutic doses of existing oncolytic viruses are significantly restricted (Aghi and Martuza, 2005). The availability of an oncolytic virus whose replication can be tightly controlled and adjusted pharmacologically would offer greatly increased safety and therapeutic efficacy. Such a regulatable oncolytic virus would minimize unwanted replication in adjacent and distant tissues as well as undesirable progeny virus overload in the target area after the tumor has been eliminated. This regulatory feature would also allow the oncolytic activity of the virus to be quickly shut down should adverse effects be detected (Aghi and Martuza, 2005; Shen and Nemunaitis, 2005). Work described herein presents a new generation of regulatable fusogenic variant of an oncolytic HSV that is significantly more effective at killing cancer cells than other oncolytic HSV viruses.

### SUMMARY OF THE INVENTION

In the current invention, we first describe the use of the HSV-2 immediate-early promoter to drive highly efficient gene expression from a reporter gene in the context of HSV-1 recombinant virus. Second, we constructed a mammalian cell expressing plasmid encoding the codon-optimized dominant-negative TGF-β mutant, mmTGF-β2-7 M developed, under the control of the modified HSV-2 ICP4 promoter. Third, we established a shuttle vector that allows efficient insertion of gene of interests into the intergenic region of the HSV-1 UL26 and UL27 genes by homologous recombination. Fourth, we constructed a tetracycline-regulatable fusogenic HSV-1 oncolytic virus that encodes mmTGF-β2-7 M under the control of the tetO-containing HSV-2 ICP4 promoter. To facilitate the secretion of mmTGF-β2-7 M, the codon-optimized mmTGF-β2-7 M is fused with the signal peptide of HSV-1 gD gene.

Accordingly, one aspect described herein provides an oncolytic Herpes Simplex Virus (HSV) comprising recombinant DNA, wherein the recombinant DNA comprises (a) a gene comprising a 5′ untranslated region and a HSV-1, or HSV-2, VP5 gene that is operably linked to an VP5 promoter comprising a TATA element; (b) a tetracycline operator sequence positioned between 6 and 24 nucleotides 3′ to said TATA element, wherein the VP5 gene lies 3′ to said tetracycline operator sequence; (c) a gene sequence encoding tetracycline repressor operably linked to an HSV immediate-early promoter, wherein the gene sequence is located at the ICP0 locus; (d) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; (e) a gene sequence encoding a functional ICP34.5 protein; and (f) a gene sequence operably linked to a modified HSV promoter, wherein the gene is located in an intergenic region of UL26 and UL27 genes, wherein said oncolytic HSV does not encode functional ICP0 and does not contain a ribozyme sequence located in said 5′ untranslated region of VP5.

One aspect described herein provides an oncolytic Herpes Simplex Virus (HSV) comprising recombinant DNA, wherein the recombinant DNA comprises (a) a gene comprising a 5′ untranslated region and a HSV-1, or HSV-2, VP5 gene that is operably linked to an VP5 promoter comprising a TATA element; (b) a tetracycline operator sequence positioned between 6 and 24 nucleotides 3′ to said TATA element, wherein the VP5 gene lies 3′ to said tetracycline operator sequence; (c) a gene sequence encoding tetracycline repressor operably linked to an HSV immediate-early promoter, wherein the gene sequence is located at the ICP0 locus; (d) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; (e) a gene sequence encoding a functional ICP34.5 protein; and (f) a gene sequence operably linked to a modified HSV promoter, wherein the gene is located in an intergenic region of UL21 and UL22 genes, wherein said oncolytic HSV does not encode functional ICP0 and does not contain a ribozyme sequence located in said 5′ untranslated region of VP5.

One aspect described herein provides an oncolytic Herpes Simplex Virus (HSV) comprising recombinant DNA, wherein the recombinant DNA comprises (a) a gene comprising a 5′ untranslated region and a HSV-1, or HSV-2, VP5 gene that is operably linked to an VP5 promoter comprising a TATA element; (b) a tetracycline operator sequence positioned between 6 and 24 nucleotides 3′ to said TATA element, wherein the VP5 gene lies 3′ to said tetracycline operator sequence; (c) a gene sequence encoding tetracycline repressor operably linked to an HSV immediate-early promoter, wherein the gene sequence is located at the ICP0 locus; (d) a variant gene that increases syncytium formation as compared to wild type, wherein the HSV-1, or HSV-2, variant gene is selected from the group consisting of a glycoprotein K (gK) variant; a glycoprotein B (gB) variant; a UL24 variant; and UL20 gene variant; (e) a gene sequence encoding a functional ICP34.5 protein; and (f) a gene sequence operably linked to a modified HSV promoter, wherein the gene is located in an intergenic region of UL21, UL22, UL26 and UL27 genes, wherein said oncolytic HSV does not encode functional ICP0 and does not contain a ribozyme sequence located in said 5′ untranslated region of VP5.

In one embodiment of any aspect herein, the gene sequence of (f) is a LacZ gene sequence.

In one embodiment of any aspect herein, the gene sequence of (f) is a dominant-negative TGF-β mutant sequence.

In one embodiment of any aspect herein, the dominant-negative TGF-β mutant sequence is a mmTGF-β2-7 M fragment sequence.

In one embodiment of any aspect herein, the promoter of (f) is a modified HSV immediate-early promoter, an HCMV immediate-early promoter, or a human elongation alpha promoter.

In one embodiment of any aspect herein, the variant gene is a gK variant gene that encodes an amino acid substitution selected from the group consisting of an Ala to Thr amino acid substitution corresponding to amino acid 40 of SEQ ID NO: 2; an Ala to “x” amino acid substitution corresponding to amino acid 40 of SEQ ID NO: 2, wherein “x” is any amino acid; an Asp to Asn amino acid substitution corresponding to amino acid 99 of SEQ ID NO: 2; a Leu to Pro amino acid substitution corresponding to amino acid 304 of SEQ ID NO: 2; and an Arg to Leu amino acid substitution corresponding to amino acid 310 of SEQ ID NO: 2.

In one embodiment of any aspect herein, the tetracycline operator sequence comprises two Op2 repressor binding sites.

In one embodiment of any aspect herein, the VP5 promoter is an HSV-1 or HSV-2 VP5 promoter.

In one embodiment of any aspect herein, the immediate-early promoter is an HSV-1 or HSV-2 immediate-early promoter.

In one embodiment of any aspect herein, the HSV immediate-early promoter is selected from the group consisting of ICP0 promoter, ICP4 promoter and ICP27 promoter.

In one embodiment of any aspect herein, the recombinant DNA is part of the HSV-1 genome.

In one embodiment of any aspect herein, the recombinant DNA is part of the HSV-2 genome.

In one embodiment of any aspect herein, the oncolytic HSV further comprising a pharmaceutically acceptable carrier.

In one embodiment of any aspect herein, the oncolytic HSV further encodes at least one polypeptide that can increase the efficacy of the oncolytic HSV to induce an anti-tumor-specific immunity.

In one embodiment of any aspect herein, the at least one polypeptide encodes a product selected from the group consisting of interleukin 2 (IL2), interleukin 12 (IL12), interleukin 15 (IL15), an anti-PD-1 antibody or antibody reagent, an anti-PD-L1 antibody or antibody reagent, an anti-OX40 antibody or antibody reagent, a CTLA-4 antibody or antibody reagent, a TIM-3 antibody or antibody reagent, a TIGIT antibody or antibody reagent, a soluble interleukin 10 receptor (IL10R), a fusion polypeptide between a soluble IL10R and IgG-Fc domain, a soluble TGFβ type II receptor (TGFBRII), a fusion polypeptide between a soluble TGFBRII and IgG-Fc domain, an anti-IL10R antibody or antibody reagent, an anti-IL10 antibody or antibody reagent, an anti-TGFBRII antibody or antibody reagent, and an anti-TGFBRII antibody or antibody reagent.

In one embodiment of any aspect herein, the oncolytic HSV the further encodes fusogenic activity.

Another aspect described herein provides an oncolytic Herpes Simplex Virus (HSV) comprising recombinant DNA, wherein the recombinant DNA comprises: (a) a gene comprising a 5′ untranslated region and a HSV-1, or HSV-2, VP5 gene that is operably linked to an VP5 promoter comprising a TATA element; (b) a tetracycline operator sequence positioned between 6 and 24 nucleotides 3′ to said TATA element, wherein the VP5 gene lies 3′ to said tetracycline operator sequence; (c) a gene sequence encoding tetracycline repressor operably linked to an HSV immediate-early promoter, wherein the gene sequence is located at the ICP0 locus; (d) a variant gene that increases syncytium formation as compared to wild type, wherein the HSV-1, or HSV-2, variant gene is selected from the group consisting of a glycoprotein K (gK) variant; a glycoprotein B (gB) variant; a UL24 variant; and UL20 gene variant; (e) a gene sequence encoding a functional ICP34.5 protein; and (f) a dominant-negative TGF-β mutant sequence operably linked to a modified HSV-2 immediate-early promoter, wherein the gene is located in an intergenic region of UL26 and UL27 genes, wherein said oncolytic HSV does not encode functional ICP0 and does not contain a ribozyme sequence located in said 5′ untranslated region of VP5.

In one embodiment of any aspect herein, the oncolytic HSV the further encodes fusogenic activity.

Another aspect described herein provides an oncolytic Herpes Simplex Virus (HSV) comprising recombinant DNA, wherein the recombinant DNA comprises: (a) a gene comprising a 5′ untranslated region and a HSV-1, or HSV-2, VP5 gene that is operably linked to an VP5 promoter comprising a TATA element; (b) a tetracycline operator sequence positioned between 6 and 24 nucleotides 3′ to said TATA element, wherein the VP5 gene lies 3′ to said tetracycline operator sequence; (c) a gene sequence encoding tetracycline repressor operably linked to an HSV immediate-early promoter, wherein the gene sequence is located at the ICP0 locus; (d) a variant gene that increases syncytium formation as compared to wild type, wherein the HSV-1, or HSV-2, variant gene is selected from the group consisting of a glycoprotein K (gK) variant; a glycoprotein B (gB) variant; a UL24 variant; and UL20 gene variant; (e) a gene sequence encoding a functional ICP34.5 protein; and (f) a dominant-negative TGF-β mutant sequence operably linked to a modified HSV-2 immediate-early promoter, wherein the gene is located in an intergenic region of UL21 and UL22 genes, wherein said oncolytic HSV does not encode functional ICP0 and does not contain a ribozyme sequence located in said 5′ untranslated region of VP5.

In one embodiment of any aspect herein, the oncolytic HSV the further encodes fusogenic activity.

Another aspect described herein provides an oncolytic Herpes Simplex Virus (HSV) comprising recombinant DNA, wherein the recombinant DNA comprises: (a) a gene comprising a 5′ untranslated region and a HSV-1, or HSV-2, VP5 gene that is operably linked to an VP5 promoter comprising a TATA element; (b) a tetracycline operator sequence positioned between 6 and 24 nucleotides 3′ to said TATA element, wherein the VP5 gene lies 3′ to said tetracycline operator sequence; (c) a gene sequence encoding tetracycline repressor operably linked to an HSV immediate-early promoter, wherein the gene sequence is located at the ICP0 locus; (d) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; (e) a gene sequence encoding a functional ICP34.5 protein; and (f) a dominant-negative TGF-β mutant sequence operably linked to a modified HSV-2 immediate-early promoter, wherein the gene is located in an intergenic region of UL21, UL22, UL26 and UL27 genes, wherein said oncolytic HSV does not encode functional ICP0 and does not contain a ribozyme sequence located in said 5′ untranslated region of VP5.

Another aspect described herein provides an oncolytic Herpes Simplex Virus (HSV) comprising recombinant DNA, wherein the recombinant DNA does not encode functional ICP0 or ICP34.5 genes; and encodes a functional mmTGF-β2-7 M fragment sequence.

An oncolytic Herpes Simplex Virus (HSV) comprising recombinant DNA, wherein the recombinant DNA does not encode functional ICP0 and ICP34.5 genes; and encodes a functional mmTGF-β2-7 M fragment sequence.

Another aspect described herein provides an oncolytic Herpes Simplex Virus (HSV) comprising recombinant DNA, wherein the recombinant DNA does not encode functional ICP0; and encodes a functional mmTGF-β2-7 M fragment sequence.

Another aspect described herein provides an oncolytic virus encoding a functional mmTGF-β2-7 M fragment sequence.

Another aspect described herein provides a recombinant virus encoding a functional mmTGF-β2-7 M fragment sequence.

Another aspect described herein provides a composition comprising any of the viruses described herein.

In one embodiment of any aspect herein, the composition further comprise a pharmaceutically acceptable carrier.

Another aspect described herein provides a cell expressing any of the viruses or compositions described herein.

In one embodiment of any aspect herein, the cell is mammalian.

In one embodiment of any aspect herein, the cell is a cancer cell or an immune cell.

In one embodiment of any aspect herein, the immune cell is a B cell or T cell.

In one embodiment of any aspect herein, the cell expresses high levels of mmTGF-β32-7 M.

Another aspect described herein provides a method for treating cancer, the method comprising administering the any of the viruses or compositions described herein to a subject having cancer.

In one embodiment of any aspect herein, the cancer is a solid tumor.

In one embodiment of any aspect herein, the tumor is benign or malignant.

In one embodiment of any aspect herein, the subject is diagnosed or has been diagnosed as having cancer is selected from the list consisting of: a carcinoma, a melanoma, a sarcoma, a germ cell tumor, and a blastoma.

In one embodiment of any aspect herein, the subject is diagnosed or has been diagnosed as having a cancer selected from the group consisting of: non-small-cell lung cancer, bladder cancer, breast cancer, brain cancer, colon cancer, prostate cancer, liver cancer, lung cancer, ovarian cancer, skin cancer, head and neck cancer, kidney cancer, and pancreatic cancer.

In one embodiment of any aspect herein, the cancer is metastatic.

In one embodiment of any aspect herein, the method further comprises administering an agent that regulates the tet operator-containing promoter.

In one embodiment of any aspect herein, the agent is doxycycline or tetracycline. In one embodiment of any aspect herein, the agent is administered locally or systemically. In one embodiment of any aspect herein, the systemic administration is oral administration.

In one embodiment of any aspect herein, the virus or composition is administered directly to the tumor.

Another aspect described herein provides a hybrid nucleic acid sequence containing a sequence of a therapeutic antibody and mmTGF-β2-7 M, wherein mmTGF-β2-7 M is fused to a Fe domain of the therapeutic antibody.

In one embodiment of any aspect herein, wherein the therapeutic antibody sequence is a sequence of an immunotherapeutic antibody.

In one embodiment of any aspect herein, the therapeutic antibody sequence is a sequence selected from the list consisting of an anti-PD-1 antibody, an anti-PD-L1 antibody, an anti-Tim3 antibody, an anti-anti-CTLA4 antibody, and anti-TDM-1 antibody, and an anti-TIGIT antibody.

Another aspect described herein provides a polypeptide encoded by any hybrid nucleic acid described herein.

Another aspect described herein provides a vector expressing any of the hybrid nucleic acids or polypeptides described herein.

Another aspect described herein provides a chimeric antigen receptor (CAR) polypeptide comprising at least one of (a) an extracellular domain comprising a dominant-negative TGF-β mutant sequence; (b) a transmembrane domain; (c) a co-stimulatory domain; and (d) an intracellular signaling domain.

Another aspect described herein provides a nucleic acid encoding the any of the CAR polypeptides described herein.

Another aspect described herein provides a mammalian cell comprising: (a) the any CAR polypeptide described herein; or any nucleic acid described herein.

In one embodiment of any aspect herein, the cell is a T cell.

In one embodiment of any aspect herein, the cell is a human cell.

In one embodiment of any aspect herein, the cell further comprises at least a second CAR polypeptide.

In one embodiment of any aspect herein, the at least second CAR polypeptide comprises an extracellular domain comprising a sequence of an immunotherapeutic antibody.

In one embodiment of any aspect herein, the cell is obtained from an individual having or diagnosed as having cancer.

Another aspect described herein provides a method of treating cancer in a subject in need thereof, the method comprising administering any of the cells described herein.

Another aspect described herein provides a method of treating cancer in a subject in need thereof, the method comprising: (a) engineering a T cell to comprise any of the CAR polypeptides described herein, or any nucleic acid described herein on the T cell surface; and (b) administering the engineered T cell to the subject.

In one embodiment of any aspect herein, the engineered T cell further comprises at least a second CAR polypeptide.

In one embodiment of any aspect herein, the method further comprises administering at least one additional anti-cancer therapeutic.

### Definitions

All references cited herein are incorporated by reference in their entirety as though fully set forth.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. Definitions of common terms can be found in Singleton et al., Dictionary of Microbiology and Molecular Biology 3^{rd} ed., J. Wiley & Sons New York, N.Y. (2001); March, Advanced Organic Chemistry Reactions, Mechanisms and Structure 5^{th} ed., J. Wiley & Sons New York, N.Y. (2001); Michael Richard Green and Joseph Sambrook, Molecular Cloning: A Laboratory Manual, 4th ed., Cold Spring Harbor Laboratory Press, Cold Spring Harbor, N.Y., USA (2012); Davis et al., Basic Methods in Molecular Biology, Elsevier Science Publishing, Inc., New York, USA (2012); Jon Lorsch (ed.) Laboratory Methods in Enzymology: DNA, Elsevier, (2013); Frederick M. Ausubel (ed.), Current Protocols in Molecular Biology (CPMB), John Wiley and Sons, (2014); John E. Coligan (ed.), Current Protocols in Protein Science (CPPS), John Wiley and Sons, Inc., (2005); and Ethan M Shevach, Warren Strobe, (eds.) Current Protocols in Immunology (CPI) (John E. Coligan, ADA M Kruisbeek, David H Margulies, John Wiley and Sons, Inc., (2003); each of which provide one skilled in the art with a general guide to many of the terms used in the present application.

“Cancer” as used herein can refer to a hyperproliferation of cells whose unique trait-loss of normal cellular control-results in unregulated growth, lack of differentiation, local tissue invasion, and metastasis, and can be leukemia, lymphoma, multiple myeloma, or a solid tumor. Non-limiting examples of leukemia include acute myeloid leukemia (AML), Chronic myeloid leukemia (CML), Acute lymphocytic leukemia (ALL), and Chronic lymphocytic leukemia (CLL). In one embodiment, the cancer is ALL or CLL. Non-limiting examples of lymphoma include Diffuse large B-cell lymphoma (DLBCL), Follicular lymphoma, Chronic lymphocytic leukemia (CLL), Small lymphocytic lymphoma (SLL), Mantle cell lymphoma (MCL), Marginal zone lymphomas, Burkitt lymphoma, hairy cell leukemia (HCL). In one embodiment, the cancer is DLBCL or Follicular lymphoma. Non-limiting examples of solid tumors include Adrenocortical Tumor, Alveolar Soft Part Sarcoma, Carcinoma, Chondrosarcoma, Colorectal Carcinoma, Desmoid Tumors, Desmoplastic Small Round Cell Tumor, Endocrine Tumors, Endodermal Sinus Tumor, Epithelioid Hemangioendothelioma, Ewing Sarcoma, Germ Cell Tumors (Solid Tumor), Giant Cell Tumor of Bone and Soft Tissue, Hepatoblastoma, Hepatocellular Carcinoma, Melanoma, Nephroma, Neuroblastoma, Non-Rhabdomyosarcoma Soft Tissue Sarcoma (NRSTS), Osteosarcoma, Paraspinal Sarcoma, Renal Cell Carcinoma, Retinoblastoma, Rhabdomyosarcoma, Synovial Sarcoma, and Wilms Tumor. Solid tumors can be found in bones, muscles, or organs, and can be sarcomas or carcinomas. It is contemplated that any aspect of the technology described herein can be used to treat all types of cancers, including cancers not listed in the instant application. As used herein, the term “tumor” refers to an abnormal growth of cells or tissues, e.g., of malignant type or benign type.

As used herein, a “subject” means a human or animal. Usually the animal is a vertebrate such as a primate, rodent, domestic animal or game animal. Primates include, for example, chimpanzees, cynomolgus monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., cancer. A subject can be male or female.

A subject can be one who has been previously diagnosed with or identified as suffering from or having a condition in need of treatment (e.g. cancer) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition or a subject who does not exhibit risk factors.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. cancer. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. ligan-mediated receptor activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Len (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Len, into Tyr or into Ile; Phe into Met, into Leu or into Tyr; Ser into Thr; Thr into Ser; Trp into Tyr; Tyr into Trp; and/or Phe into Val, into Ile or into Len.

In some embodiments, a polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to an assay known in the art or described below herein. A functional fragment can comprise conservative substitutions of the sequences disclosed herein.

In some embodiments, a polypeptide described herein can be a variant of a polypeptide or molecule as described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

As used herein, the term “DNA” is defined as deoxyribonucleic acid. The term “polynucleotide” is used herein interchangeably with “nucleic acid” to indicate a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The term “operably linked,” as used herein, refers to the arrangement of various nucleic acid molecule elements relative to each other such that the elements are functionally connected and are able to interact with each other. Such elements may include, without limitation, a promoter, an enhancer, a polyadenylation sequence, one or more introns and/or exons, and a coding sequence of a gene of interest to be expressed. The nucleic acid sequence elements, when operably linked, can act together to modulate the activity of one another, and ultimately may affect the level of expression of the gene of interest, including any of those encoded by the sequences described above.

The term “vector,” as used herein, refers to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both of which are incorporated herein by reference). Additionally, the techniques described herein and demonstrated in the referenced figures are also instructive with regard to effective vector construction.

The term “oncolytic HSV-1 vector” refers to a genetically engineered HSV-1 virus corresponding to at least a portion of the genome of HSV-1 that is capable of infecting a target cell, replicating, and being packaged into HSV-1 virions. The genetically engineered virus comprises deletions and or mutations and or insertions of nucleic acid that render the virus oncolytic such that the engineered virus replicates in- and kills—tumor cells by oncolytic activity. The virus may be attenuated or non-attenuated. The virus may or may not deliver a transgene—that differs from the HSV viral genome. In one embodiment, the oncolytic HSV-1 vector does not express a transgene to produce a protein foreign to the virus.

As used herein, “UL21” refers to tegument protein UL21 (e.g., from Human alphaherpesvirus 1). Sequences for UL21, are known for a number of species, e.g., HSV-1 UL21 (NCBI Gene ID: 2703372) polypeptide (e.g., NCBI Ref Seq YP_009137095.1). UL21 can refer to HSV-1 UL21, including naturally occurring variants, molecules, and alleles thereof, and can refer to homologs, such as HSV-2.

As used herein, “UL22” refers to envelope glycoprotein H (e.g., from Human alphaherpesvirus 1). Sequences for UL22, are known for a number of species, e.g., HSV-1 UL22 (NCBI Gene ID: 24271466) polypeptide (e.g., NCBI Ref Seq YP_009137096.1). UL22 can refer to HSV-1 UL22, including naturally occurring variants, molecules, and alleles thereof, and can refer to homologs, such as HSV-2.

As used herein, “UL26” refers to a capsid maturation protease (e.g., from Human alphaherpesvirus 1). Sequences for UL26, are known for a number of species, e.g., HSV-1 UL26 (NCBI Gene ID: 2703453) polypeptide (e.g., NCBI Ref Seq YP_009137100.1). UL26 can refer to HSV-1 UL26, including naturally occurring variants, molecules, and alleles thereof, and can refer to homologs, such as HSV-2.

As used herein, “UL27” refers to envelope glycoprotein B (e.g., from Human alphaherpesvirus 1). Sequences for UL27, are known for a number of species, e.g., HSV-1 UL27 (NCBI Gene ID: 24271469) polypeptide (e.g., NCBI Ref Seq YP_009137102.1). UL27 can refer to HSV-1 UL27, including naturally occurring variants, molecules, and alleles thereof, and can refer to homologs, such as HSV-2.

The term “promoter,” as used herein, refers to a nucleic acid sequence that regulates, either directly or indirectly, the transcription of a corresponding nucleic acid coding sequence to which it is operably linked. The promoter may function alone to regulate transcription, or, in some cases, may act in concert with one or more other regulatory sequences such as an enhancer or silencer to regulate transcription of the gene of interest. The promoter comprises a DNA regulatory sequence, wherein the regulatory sequence is derived from a gene, which is capable of binding RNA polymerase and initiating transcription of a downstream (3′-direction) coding sequence. A promoter generally comprises a sequence that functions to position the start site for RNA synthesis. The best-known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one can position the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. Depending on the promoter used, individual elements can function either cooperatively or independently to activate transcription. The promoters described herein may or may not be used in conjunction with an “enhancer,” which refers to a cis-acting regulatory sequence involved in the transcriptional activation of a nucleic acid sequence, such as those for the genes, or portions or functional equivalents thereof, listed herein.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages may be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include, the HCMV immediate-early promoter, the beta-lactamase (penicillinase), lactose and tryptophan (trp) promoter systems.

A “gene,” or a “sequence which encodes” a particular protein, is a nucleic acid molecule which is transcribed (in the case of DNA) and translated (in the case of mRNA) into a polypeptide in vitro or in vivo when placed under the control of one or more appropriate regulatory sequences. A gene of interest can include, but is no way limited to, cDNA from eukaryotic mRNA, genomic DNA sequences from eukaryotic DNA, and even synthetic DNA sequences. A transcription termination sequence will usually be located 3′ to the gene sequence. Typically, a polyadenylation signal is provided to terminate transcription of genes inserted into a recombinant virus.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

The term “transgene” refers to a particular nucleic acid sequence encoding a polypeptide or a portion of a polypeptide to be expressed in a cell into which the nucleic acid sequence is inserted. The term “transgene” is meant to include (1) a nucleic acid sequence that is not naturally found in the cell (i.e., a heterologous nucleic acid sequence); (2) a nucleic acid sequence that is a mutant form of a nucleic acid sequence naturally found in the cell into which it has been inserted; (3) a nucleic acid sequence that serves to add additional copies of the same (i.e., homologous) or a similar nucleic acid sequence naturally occurring in the cell into which it has been inserted; or (4) a silent naturally occurring or homologous nucleic acid sequence whose expression is induced in the cell into which it has been inserted. A “mutant form” or “modified nucleic acid” or “modified nucleotide” sequence means a sequence that contains one or more nucleotides that are different from the wild-type or naturally occurring sequence, i.e., the mutant nucleic acid sequence contains one or more nucleotide substitutions, deletions, and/or insertions. In some cases, the gene of interest may also include a sequence encoding a leader peptide or signal sequence such that the transgene product may be secreted from the cell.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like.

The term “oncolytic activity,” as used herein, refers to cytotoxic effects in vitro and/or in vivo exerted on tumor cells without any appreciable or significant deleterious effects to normal cells under the same conditions. The cytotoxic effects under in vitro conditions are detected by various means as known in prior art, for example, by staining with a selective stain for dead cells, by inhibition of DNA synthesis, or by apoptosis. Detection of the cytotoxic effects under in vivo conditions is performed by methods known in the art.

A “biologically active” portion of a molecule, as used herein, refers to a portion of a larger molecule that can perform a similar function as the larger molecule. Merely by way of non-limiting example, a biologically active portion of a promoter is any portion of a promoter that retains the ability to influence gene expression, even if only slightly. Similarly, a biologically active portion of a protein is any portion of a protein which retains the ability to perform one or more biological functions of the full-length protein (e.g. binding with another molecule, phosphorylation, etc.), even if only slightly.

As used herein, the term “administering,” refers to the placement of a therapeutic or pharmaceutical composition as disclosed herein into a subject by a method or route which results in at least partial delivery of the agent at a desired site. Pharmaceutical compositions comprising agents as disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±10%.

As used herein, the term “comprising” means that other elements can also be present in addition to the defined elements presented. The use of “comprising” indicates inclusion rather than limitation. The term “consisting of” refers to compositions, methods, and respective components thereof as described herein, which are exclusive of any element not recited in that description of the embodiment. As used herein the term “consisting essentially of” refers to those elements required for a given embodiment. The term permits the presence of additional elements that do not materially affect the basic and novel or functional characteristic(s) of that embodiment of the technology.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

With the aforementioned preliminary descriptions and definitions in mind, additional background is provided herein below to provide context for the genesis and development of the inventive vectors, compositions and methods described herein.

### BRIEF DESCRIPTION OF THE DRAWINGS

Exemplary embodiments are illustrated in the referenced figures. It is intended that the embodiments and figures disclosed herein are to be considered illustrative rather than restrictive.

FIG. 1 shows a schematic diagram of the genome of QREOF-lacZ. UL and US represent the unique long and unique short regions of the HSV-1 genome, respectively, which are flanked by their corresponding inverted repeat regions (open boxes). The replacement of the ICP0 coding sequences with DNA sequences encoding tetR (black box) and intron II of the rabbit β-globin gene (obliquely striped box) flanked by ICP0 sequences are shown above the diagram of the HSV-1 genome. ICP5TO indicates HSV-1 UL19 gene that encodes ICP5 under the control of the tetO-bearing HSV-1 ICP5 promoter. An expanded DNA fragment, inserted at the intergenic region of the UL26 gene and UL27 gene, which encodes the lacZ gene under the control of the modified HSV-2 ICP0 promoter (cross-hatched box).

FIG. 2 shows H1299 cells seeded at 7.5×10e5 cells per 60 mm dish. Cells were infected with QREOF-lacZ at an MOI of 0.05 PFU/dish at 48 h post-cell seeding in the presence or absence of doxycycline. Infected cells were stained with X-Gal at 48 h post-infection and photographed.

FIG. 3 shows U2OS cells seeded at 1.5×10e6 cells/dish. At 20 h post-seeding, duplicate dishes of cells were transfected with either 5 ug/dish of pICP6-eGFP or 5 ug/dish of pQUL2627-TGF-DN by lipofectamine 2000. Cell extracts were prepared at 70 h post-transfection. Proteins in transfected cell extracts and medium collected from transfected cells were resolved on SDS-PAGE, followed by immunoblotting with rabbit anti-TGF-β1 antibody (Abeam, ab179695).

FIG. 4 shows effect of blocking TGF-β1 signaling by mmTGF-β2-7 M on the proliferation of U2OS cells. U2OS cells were seeded at 1.5×10e6 cells/dish. At 20 h post-seeding, triplicate dishes of cells were transfected with either 5 ug/dish of pICP6-EGFP or 5 ug/dish of pQUL2627-TGFDN by lipofectamine 2000. Cells were harvested at 76 h post-transfection. Viable cells were counted by trypan blue exclusion and graphed as number of viable cells per dish, expressed as means±SEM.

FIG. 5 shows expression of mmTGF-β2-7 M in QREOF-DNT-infected U2OS cells. U2OS cells in triplicate were either mock-infected or infected with QREOF-DNT, or QREOF-lacZ at an MOI of 3 PFU/cell in the presence of doxycycline. Infected cell extracts were prepared at 18 h post-infection. Proteins in mock-infected and infected cell extracts were resolved on SDS-PAGE, followed by immunoblotting with monoclonal antibodies against HSV-1 ICP27 (Santa Cruz) or rabbit anti-TGF-01 antibody.

FIG. 6 shows a schematic of QREO5F genome.

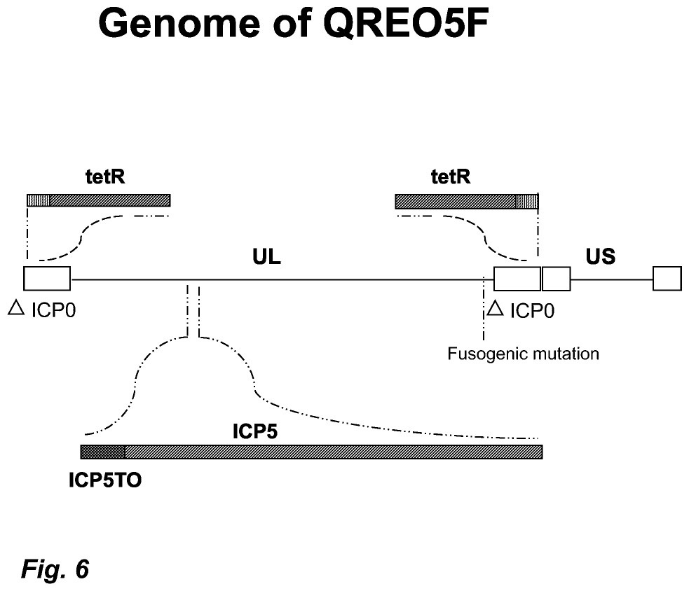

### DESCRIPTION OF THE INVENTION

Oncolytic viruses are genetically modified viruses that preferentially replicate in host cancer cells, leading to the production of new viruses and ultimately, cell death. Herpes simplex virus (HSV) possesses several unique properties as an oncolytic agent. It can infect a broad range of cell types and has a short replication cycle (9 to 18 h). The use of a replication-conditional strain of HSV-1 as an oncolytic agent was first reported for the treatment of malignant gliomas. Since then, various efforts have been made in an attempt to broaden their therapeutic efficacy and increase the replication specificity of the virus in tumor cells. Not surprisingly, however, deletion of genes that impair viral replication in normal cells also leads to a marked decrease in the oncolytic activity of the virus for the targeted tumor cells. Currently, no oncolytic viruses that are able to kill only tumor cells while leaving normal cells intact are available. Consequently, the therapeutic doses of existing oncolytic viruses are significantly restricted. The availability of an oncolytic virus whose replication can be tightly controlled and adjusted pharmacologically would offer greatly increased safety and therapeutic efficacy. Such a regulatable oncolytic virus would minimize the risk of uncontrolled replication in adjacent and distant tissues as well as undesirable progeny virus overload in the target area after the tumor has been eliminated. This regulatory feature would also allow the oncolytic activity of the virus to be quickly shut down should adverse effects be detected.

### Oncolytic HSV

HSV replicates in epithelial cells and fibroblasts and establishes life-long latent infection in neuronal cell bodies within the sensory ganglia of infected individuals. During productive infection, HSV genes fall into three major classes based on the temporal order of their expression: immediate-early (IE), early (E), and late (L) (Roizman, 2001). The HSV-1 viral proteins directly relevant to the current invention are immediate-early regulatory protein, ICP0, and the viral major capsid protein ICP5 or VP5. Although not essential for productive infection, ICP0 is required for efficient viral gene expression and replication at low multiplicities of infection in normal cells and efficient reactivation from latent infection (Cai and Schaffer, 1989; Leib et al., 1989; Yao and Schaffer, 1995). ICP0 is needed to stimulate translation of viral mRNA in quiescent cells (Walsh and Mohr, 2004) and plays a fundamental role in counteracting host innate antiviral response to HSV infection. In brief, it prevents an IFN-induced nuclear block to viral transcription, down regulates TLR2/TLR9-induced inflammatory cytokine response to viral infection, suppresses TNF-α mediated activation of NF-κB signaling pathway, and interferes with DNA damage response to viral infection (Lanfranca et al., 2014). Given that tumor cells are impaired in various cellular pathways, such as DNA repair, interferon signaling, and translation regulation (Barber, 2015; Critchley-Thorne et al., 2009; Kastan and Bartek, 2004; Li and Chen, 2018; Mohr, 2005; Zitvogel et al., 2015), it is not surprising that ICP0 deletion mutants replicate much more efficiently in cancer cells than in normal cells, in particular, quiescent cells and terminally differentiated cells. The oncolytic potential of ICP0 mutants was first illustrated by Yao and Schaffer (Yao and Schaffer, 1995), who showed that the plaque-forming efficiency of an ICP0 null mutant in human osteosarcoma cells (U2OS) is 100- to 200-fold higher than in non-tumorigenic African green monkey kidney cells (Vero). It has been recently shown the defect in stimulator of interferon genes (STING) signaling pathway in U2OS cells leads to its demonstrated ability to efficiently support the growth of ICP0 null mutant (Deschamps and Kalamvoki, 2017).

Using the T-REx™ gene switch technology (Thermo Fisher/Invitrogen, Carlsbad, Calif.) invented by Dr. Feng Yao and a self-cleaving ribozyme, the first regulatable oncolytic virus, KTR27 (U.S. Pat. No. 8,236,941, which is incorporated herein by reference in its entirety), in which the HSV-1 ICP0 gene is replaced by DNA sequence encoding tetracycline repressor (tetR) was created, while the essential HSV-1 ICP27 gene is controlled by the tetO-bearing ICP27 promoter and a self-cleaving ribozyme in the 5′ untranslated region of the ICP27 coding sequence. Recent DNA sequence analyses of a KTR27-derived fusogenic virus, named KTR27-F, indicates that in addition to the deletion of both copies of ICP0 gene, both copies of HSV-1 ICP34.5 gene are also deleted from the said KTR27-F virus. Moreover, PCR analyses of KTR27 viral DNA with the ICP34.5 gene-specific primers has revealed that like KTR27-F, KTR27 does not encode ICP0 gene and ICP34.5 gene. ICP34.5 gene is located 5′ to the ICP0 gene in the inverted repeat region of HSV-1 genome that flanks the unique long sequence of HSV-1 genome. Various HSV-1 oncolytic viruses are based on the deletion of ICP34.5 gene (Aghi and Martuza, 2005; Kaur et al., 2012; Lawler et al., 2017), including the recently FDA-approved talimogene laherparepvec (T-VEC) for treatment of advanced-stage melanoma (Rehman et al., 2016).

Building on the tet-dependent viral replication and onco-selectivity profiles of KTR27 and the notion that the self-cleaving ribozyme employed in construction of KTR27 for achieving higher degree of tet-dependent viral replication significantly restricts viral replication in cancer cells because of less than optimal expression of ICP27, a new ICP0 null mutant-based tetR-expressing oncolytic virus QREO5 that encodes the late HSV-1 major capsid protein VP5 under the control of the tetO-containing VP5 promoter was recently developed. Because VP5 is a late viral gene product, whose expression is dependent on the expression of viral IE genes, it was hypothesized that the late kinetics of the tetO-bearing VP5 promoter would allow for more stringent control of VP5 expression than that of ICP27 under the control of the tetO-bearing ICP27 promoter by tetR expressed from the IE ICP0 promoter. Indeed, QREO5 exhibits significantly superior tet-dependent viral replication than KTR27 in infected H1299 cells and Vero cells. Moreover, because the QREO5 genome contains no self-cleaving ribozyme and encodes wild-type ICP34.5 gene, it replicates 100- and 450-fold more efficiently than KTR27 in Vero cells and H1299 cells, respectively.

HSV-1 is a human neurotropic virus that is capable of infecting virtually all vertebrate cells. Natural infections follow either a lytic, replicative cycle or establish latency, usually in peripheral ganglia, where the DNA is maintained indefinitely in an episomal state. HSV-1 contains a double-stranded, linear DNA genome, about 152 kilobases in length, which has been completely sequenced by McGeoch (McGeoch et al., J. Gen. Virol. 69: 1531 (1988); McGeoch et al., Nucleic Acids Res 14: 1727 (1986); McGeoch et al., J. Mol. Biol. 181: 1 (1985); Perry and McGeoch, J. Gen. Virol. 69:2831 (1988); Szpara M L et al., J Virol. 2010, 84:5303; Macdonald S J et al., J Virol. 2012, 86:6371). DNA replication and virion assembly occurs in the nucleus of infected cells. Late in infection, concatemeric viral DNA is cleaved into genome length molecules which are packaged into virions. In the CNS, herpes simplex virus spreads transneuronally followed by intraaxonal transport to the nucleus, either retrograde or anterograde, where replication occurs.

Accordingly, one aspect described herein provides an oncolytic Herpes Simplex Virus (HSV) comprising recombinant DNA, wherein the recombinant DNA comprises (a) a gene comprising a 5′ untranslated region and a HSV-1, or HSV-2, VP5 gene that is operably linked to an VP5 promoter comprising a TATA element; (b) a tetracycline operator sequence positioned between 6 and 24 nucleotides 3′ to said TATA element, wherein the VP5 gene lies 3′ to said tetracycline operator sequence; (c) a gene sequence encoding tetracycline repressor operably linked to an HSV immediate-early promoter, wherein the gene sequence is located at the ICP0 locus; (d) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; (e) a gene sequence encoding a functional ICP34.5 protein; and (f) a gene sequence operably linked to a modified HSV promoter, wherein the gene is located in an intergenic region of UL26 and UL27 genes, wherein said oncolytic HSV does not encode functional ICP0 and does not contain a ribozyme sequence located in said 5′ untranslated region of VP5.

One aspect described herein provides an oncolytic Herpes Simplex Virus (HSV) comprising recombinant DNA, wherein the recombinant DNA comprises (a) a gene comprising a 5′ untranslated region and a HSV-1, or HSV-2, VP5 gene that is operably linked to an VP5 promoter comprising a TATA element; (b) a tetracycline operator sequence positioned between 6 and 24 nucleotides 3′ to said TATA element, wherein the VP5 gene lies 3′ to said tetracycline operator sequence; (c) a gene sequence encoding tetracycline repressor operably linked to an HSV immediate-early promoter, wherein the gene sequence is located at the ICP0 locus; (d) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; (e) a gene sequence encoding a functional ICP34.5 protein; and (f) a gene sequence operably linked to a modified HSV promoter, wherein the gene is located in an intergenic region of UL21 and UL22 genes, wherein said oncolytic HSV does not encode functional ICP0 and does not contain a ribozyme sequence located in said 5′ untranslated region of VP5.

One aspect described herein provides an oncolytic Herpes Simplex Virus (HSV) comprising recombinant DNA, wherein the recombinant DNA comprises (a) a gene comprising a 5′ untranslated region and a HSV-1, or HSV-2, VP5 gene that is operably linked to an VP5 promoter comprising a TATA element; (b) a tetracycline operator sequence positioned between 6 and 24 nucleotides 3′ to said TATA element, wherein the VP5 gene lies 3′ to said tetracycline operator sequence; (c) a gene sequence encoding tetracycline repressor operably linked to an HSV immediate-early promoter, wherein the gene sequence is located at the ICP0 locus; (d) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant; (e) a gene sequence encoding a functional ICP34.5 protein; and (f) a gene sequence operably linked to a modified HSV promoter, wherein the gene is located in an intergenic region of UL21, UL22, UL26 and UL27 genes, wherein said oncolytic HSV does not encode functional ICP0 and does not contain a ribozyme sequence located in said 5′ untranslated region of VP5.

Another aspect described herein provides an oncolytic Herpes Simplex Virus (HSV) comprising recombinant DNA, wherein the recombinant DNA comprises: (a) a gene comprising a 5′ untranslated region and a HSV-1, or HSV-2, VP5 gene that is operably linked to an VP5 promoter comprising a TATA element; (b) a tetracycline operator sequence positioned between 6 and 24 nucleotides 3′ to said TATA element, wherein the VP5 gene lies 3′ to said tetracycline operator sequence; (c) a gene sequence encoding tetracycline repressor operably linked to an HSV immediate-early promoter, wherein the gene sequence is located at the ICP0 locus; (d) a variant gene that increases syncytium formation as compared to wild type, wherein the HSV-1, or HSV-2, variant gene is selected from the group consisting of a glycoprotein K (gK) variant; a glycoprotein B (gB) variant; a UL24 variant; and UL20 gene variant; (e) a gene sequence encoding a functional ICP34.5 protein; and (f) a dominant-negative TGF-β mutant sequence operably linked to a modified HSV-2 immediate-early promoter, wherein the gene is located in an intergenic region of UL26 and UL27 genes, wherein said oncolytic HSV does not encode functional ICP0 and does not contain a ribozyme sequence located in said 5′ untranslated region of VP5.

Another aspect described herein provides an oncolytic Herpes Simplex Virus (HSV) comprising recombinant DNA, wherein the recombinant DNA comprises: (a) a gene comprising a 5′ untranslated region and a HSV-1, or HSV-2, VP5 gene that is operably linked to an VP5 promoter comprising a TATA element; (b) a tetracycline operator sequence positioned between 6 and 24 nucleotides 3′ to said TATA element, wherein the VP5 gene lies 3′ to said tetracycline operator sequence; (c) a gene sequence encoding tetracycline repressor operably linked to an HSV immediate-early promoter, wherein the gene sequence is located at the ICP0 locus; (d) a variant gene that increases syncytium formation as compared to wild type, wherein the HSV-1, or HSV-2, variant gene is selected from the group consisting of a glycoprotein K (gK) variant; a glycoprotein B (gB) variant; a UL24 variant; and UL20 gene variant; (e) a gene sequence encoding a functional ICP34.5 protein; and (f) a dominant-negative TGF-β mutant sequence operably linked to a modified HSV-2 immediate-early promoter, wherein the gene is located in an intergenic region of UL21 and UL22 genes, wherein said oncolytic HSV does not encode functional ICP0 and does not contain a ribozyme sequence located in said 5′ untranslated region of VP5.

Another aspect described herein provides an oncolytic Herpes Simplex Virus (HSV) comprising recombinant DNA, wherein the recombinant DNA comprises: (a) a gene comprising a 5′ untranslated region and a HSV-1, or HSV-2, VP5 gene that is operably linked to an VP5 promoter comprising a TATA element; (b) a tetracycline operator sequence positioned between 6 and 24 nucleotides 3′ to said TATA element, wherein the VP5 gene lies 3′ to said tetracycline operator sequence; (c) a gene sequence encoding tetracycline repressor operably linked to an HSV immediate-early promoter, wherein the gene sequence is located at the ICP0 locus; (d) a variant gene that increases syncytium formation as compared to wild type, wherein the HSV-1, or HSV-2, variant gene is selected from the group consisting of a glycoprotein K (gK) variant; a glycoprotein B (gB) variant; a UL24 variant; and UL20 gene variant; (e) a gene sequence encoding a functional ICP34.5 protein; and (f) a dominant-negative TGF-β mutant sequence operably linked to a modified HSV-2 immediate-early promoter, wherein the gene is located in an intergenic region of UL21, UL22, UL26 and UL27 genes, wherein said oncolytic HSV does not encode functional ICP0 and does not contain a ribozyme sequence located in said 5′ untranslated region of VP5.

A distinguishing feature of the oncolytic virus described herein is that the viral genome expression a gene sequence that encodes functional ICP34.5. Infected cell protein 34.5 (ICP34.5) is a protein (e.g., a gene product) expressed by the γ34.5 gene in viruses, such as the herpes simplex virus. ICP34.5 is one of HSV neurovirulence factors (Chou J, Kern E R, Whitley R J, and Roizman B, Science, 1990). One of the functions of ICP34.5 is to block the cellar stress response to a viral infection, i.e., blocking the double-stranded RNA-dependent protein kinase PKR-mediated antiviral response (Agarwalla, P. K., et al. Method in Mol. Bio., 2012).

The oncolytic virus described herein is a ICP0 null virus. Infected cell polypeptide 0 (ICP0) is a protein encoded by the HSV-1 α0 gene. ICP0 is generated during the immediate-early phase of viral gene expression. ICP0 is synthesized and transported to the nucleus of the infected host cell, where it promotes transcription from viral genes, disrupts nuclear and cytoplasmic cellular structures, such as the microtubule network, and alters the expression of host genes. One skilled in the art can determine if the ICP0 gene product has been deleted or if the virus does not express functional forms of this gene product using PCR-based assays to detect the presence of the gene in the viral genome or the expression of the gene products, or using functional assays to assess their function, respectively.

In one embodiment, the gene that encodes these gene products contain a mutation, for example, an inactivating mutation, that inhibits proper expression of the gene product. For example, the gene may encode a mutation in the gene product that inhibits proper folding, expression, function, etc. of the gene product. As used herein, the term “inactivating mutation” is intended to broadly mean a mutation or alteration to a gene wherein the expression of that gene is significantly decreased, or wherein the gene product is rendered nonfunctional, or its ability to function is significantly decreased. The term “gene” encompasses both the regions coding the gene product as well as regulatory regions for that gene, such as a promoter or enhancer, unless otherwise indicated.

Ways to achieve such alterations include: (a) any method to disrupt the expression of the product of the gene or (b) any method to render the expressed gene nonfunctional. Numerous methods to disrupt the expression of a gene are known, including the alterations of the coding region of the gene, or its promoter sequence, by insertions, deletions and/or base changes. (See, Roizman, B. and Jenkins, F. J., Science 229: 1208-1214 (1985)).

An essential feature of the DNA of the present invention is the presence of a gene needed for virus replication that is operably linked to a promoter having a TATA element. A tet operator sequence is located between 6 and 24 nucleotides 3′ to the last nucleotide in the TATA element of the promoter and 5′ to the gene. The strength with which the tet repressor binds to the operator sequence is enhanced by using a form of operator which contains two op2 repressor binding sites (each such site having the nucleotide sequence: TCCCTATCAGTGATAGAGA (SEQ ID NO: 8)) linked by a sequence of 2-20, preferably 1-3 or 10-13, nucleotides. When repressor is bound to this operator, very little or no transcription of the associated gene will occur. If DNA with these characteristics is present in a cell that also expresses the tetracycline repressor, transcription of the gene will be blocked by the repressor binding to the operator and replication of the virus will not occur. However, if tetracycline, for example, is introduced, it will bind to the repressor, cause it to dissociate from the operator, and virus replication will proceed.

During productive infection, HSV gene expression falls into three major classes based on the temporal order of expression: immediate-early (α), early (β), and late (γ), with late genes being further divided into two groups, γ1 and γ2. The expression of immediate-early genes does not require de novo viral protein synthesis and is activated by the virion-associated protein VP16 together with cellular transcription factors when the viral DNA enters the nucleus. The protein products of the immediate-early genes are designated infected cell polypeptides ICP0, ICP4, ICP22, ICP27, and ICP47 and it is the promoters of these genes that are preferably used in directing the expression of tet repressor (tetR). The expression of a gene needed for virus replication is under the control of the tetO-containing promoters and these essential genes may be immediate-early, early or late genes, e.g., ICP4, ICP27, ICP8, UL9, gD and VP5. In one embodiment, the tetR has the sequence of SEQ ID NO: 9.

ICP0 plays a major role in enhancing the reactivation of HSV from latency and confers a significant growth advantage on the virus at low multiplicities of infection. ICP4 is the major transcriptional regulatory protein of HSV-1, which activates the expression of viral early and late genes. ICP27 is essential for productive viral infection and is required for efficient viral DNA replication and the optimal expression of subset of viral β genes and γ1 genes as well as viral γ2 genes. The function of ICP47 during HSV infection appears to be to down-regulate the expression of the major histocompatibility complex (MHC) class I on the surface of infected cells.

The recombinant DNA may also include at least one, and preferably at least two, sequences coding for the tetracycline repressor with expression of these sequences being under the control of an immediate early promoter, preferably ICP0 or ICP4. The sequence for the HSV ICP0, ICP4 and ICP27 promoters and for the genes whose regulation they endogenously control are well known in the art (Perry, et al., J. Gen. Virol. 67:2365-2380 (1986); McGeoch et al., J. Gen. Virol. 72:3057-3075 (1991); McGeoch et al., Nucl. Acid Res. 14:1727-1745 (1986)) and procedures for making viral vectors containing these elements have been previously described (see U.S. published application 2005-0266564).

These promoters are not only very active in promoting gene expression, they are also specifically induced by VP16, a transactivator released when HSV-1 infects a cell. Thus, transcription from ICP0 promoter is particularly high when repressor is most needed to shut down virus replication. Once appropriate DNA constructs have been produced, they may be incorporated into HSV-1 virus using methods that are well known in the art. One appropriate procedure is described in U.S. 2005-0266564 but other methods known in the art may also be employed.

Additional promoters that can be utilized in the present inventor to drive gene expression in (f) include, but are not limited to, a modified HSV immediate-early promoter (e.g., the HSV ICP0, ICP4, ICP27, ICP22 and ICP47 promoter/regulatory sequences), an HCMV immediate-early promoter (e.g., pWRG7128 (Roy et al, Vaccine 19, 764-778, 2001), and pBC12/CMV and pJW4303 which are mentioned in WO 95/20660; which are incorporated herein by reference in their entities), or a human elongation factor-1 alpha (EF-1 alpha) promoter. These promotors are known in the art and a skilled person would be able identify the sequence of these promoters to be used. In one embodiment, the promoter of (f) is a HSV-2 immediate early promoter having a tet operator-containing.

In various embodiments, the variant gene comprises at least one amino acid change that deviates from the wild-type sequence of the gene. In one embodiment, an oncolytic HSV described herein can contain two or more amino acid substitutions in at least one variant gene. The at least two amino acid substitutions can be found in the same gene, for example, the gK variant gene contains at least two amino acid substitutions. Alternatively, the at least two amino acid substitutions can be found in the at least two different genes, for example, the gK variant gene and the UL24 variant gene each contains at least one amino acid substitutions.

SEQ ID NO: 2 is the amino acid sequence encoding
gK (strain KOS).
(SEQ ID NO: 2)
MLAVRSLQHLSTWLITAYGLVLVWYTVFGASPLHRCIYAVRPT
GTNNDTALVWMKMNQTLLFLGAPTHPPNGGWRNHAHICYANLIAGRVVP
FQVPPDATNRRIMNVHEAVNCLETLWYTRVRLVVVGWFLYLAFVALHQR
RCMFGVVSPAHKMVAPATYLLNYAGRIVSSVFLQYPYTKITRLLCELSV
QRQNLVQLFETDPVTFLYHRPAIGVIVGCELMLRFVAVGLIVGTAFISR
GACAITYPLFLTITTWCFVSTIGLTELYCILRRGPAPKNADKAAAPGRS
KGLSGVCGRCCSIILSGIAMRLCYIAWAGWLVALHYEQEIQRRLFDV

Another distinguishing feature of the oncolytic virus described herein is that the viral genome sequence does not contain a ribozyme sequence, for example, at the 5′ untranslated region of VP5. A ribozyme is an RNA molecule that is capable of catalyzing a biochemical reaction in a similar manner as a protein enzyme. Ribozymes are further described in, e.g., Yen et al., Nature 431:471-476, 2004, the contents of which are incorporated herein by reference in its entirety.

In one embodiment of various aspects, the oncolytic virus expresses a LacZ gene, which is well known in the art.

In one embodiment of various aspects, the oncolytic virus expresses a dominant negative TGFβ. As used herein, the term “dominant negative” refers to a mutated or modified protein that substantially prevents the corresponding protein having wild-type function from performing the wild-type function. For example, a dominate negative TGFβ will be capable of inhibiting the wild-type function of TGFβ in a cell that expresses the dominant negative.

In one embodiment, the dominant negative TGFβ is capable of inhibiting function (e.g., capacity to initiate TGFβ signaling) or expression levels (e.g., mRNA or protein levels) of wild-type TGFβ by at least 10%. In one embodiment, the dominant negative TGFβ is capable of inhibiting wild-type function (e.g., capacity to initiate TGFβ signaling) or expression levels (e.g., mRNA or protein levels) by at least 5%, 10%, 15%, 20%, 25%, 30%, 35%, 40%, 45%, 50%, 55%, 60%, 65%, 70%, 75%, 80%, 85%, 90%, 95%, 99% or more as compared to an appropriate control. As used herein, an appropriate control refers to the function or expression level of TGFβ in a cell that was not contacted by the dominate negative TGFβ. One skilled in the art can assess the function of TGFβ by, e.g., assessing the level of TGFβ signaling in the cell, or the expression level of TGFβ by, e.g., western blotting or PCR-based assays to assess the protein and mRNA levels, respectively.

In one embodiment, the dominant negative TGFβ comprises, consists of, or consists essentially of at least 90% sequence identity to wild-type TGFβ, and is capable of inhibiting the wild-type function of TGFβ. In another embodiment, the dominant negative TGFβ comprises, consists of, or consists essentially of at least 50%, 55%, 60%, 65%, 70%, 75%, 80%, 85%, 90%, 95%, 99%, or more sequence identity to wild-type TGFβ, and is capable of inhibiting the wild-type function of TGFβ.

In one embodiment, the dominant negative TGFβ is mmTGF-β2-7 M fragment, having a nucleotide sequence of SEQ ID NO: 10.

(SEQ ID NO: 10)
ATGGCCCTGGACGCCGCCTACTGCTTCCGCAACGTGCAGGACAACTGCT
GCCTGCGCCCCCTGTACATCGACTTCCGCAAGGACCTGGGCTGGAAGTG
GATCCACGAGCCCAAGGGCTACAACGCCAACTTCTGCGCCGGCGCCTGC
CCCTACCGCGCCAGCAAGAGCCCCAGCTGCGTGAGCCAGGACCTGGAGC
CCCTGACCATCGTGTACTACGTGGGCCGCAAGCCCAAGGTGGAGCAGCT
GAGCAACATGATCGTGAAGAGCTGCAAGTGCAGCTAA .

In one embodiment, the dominant negative TGFβ is mmTGF-β2-7 M fragment, having an amino acid sequence of SEQ ID NO: 11.

(SEQ ID NO 11)
ALDAAYCFRN VQDNCCLRPL YIDFRKDLGW KWIHEPKGYN
ANFCAGACPY
RASKSPSCVS QDLEPLTIVY YVGRKPKVEQ LSNMIVKSCK CS.

In one embodiment, the dominant negative TGFβ comprises, consists of, or consists essentially of at least 50%, 55%, 60%, 65%, 70%, 75%, 80%, 85%, 90%, 95%, 99%, or more sequence identity to SEQ ID NO: 10 or SEQ ID NO: 11.

In one embodiment, the oncolytic HSV described herein further comprises at least one polypeptide that encodes a product (e.g., a protein, a gene, a gene product, or an antibody or antibody reagent) that can increase the efficacy of the oncolytic HSV to induce an anti-tumor-specific immunity. Exemplary products include, but are not limited to, interleukin 2 (IL2), interleukin 12 (IL12), interleukin 15 (IL15), an anti-PD-1 antibody or antibody reagent, an anti-PD-L1 antibody or antibody reagent, an anti-OX40 antibody or antibody reagent, a CTLA-4 antibody or antibody reagent, a TIM-3 antibody or antibody reagent, a TIGIT antibody or antibody reagent, a soluble interleukin 10 receptor (IL10R), a fusion polypeptide between a soluble IL10R and IgG-Fc domain, a soluble TGF-β type II receptor (TGFBRII), a fusion polypeptide between a soluble TGFBRII and IgG-Fc domain, an anti-IL10R antibody or antibody reagent, an anti-IL10 antibody or antibody reagent, an anti-TGF-β1 antibody or antibody reagent, and an anti-TGFBRII antibody or antibody reagent. In one embodiment, the product is a fragment of IL-2, IL-12, or IL-15, that comprises the same functionality of IL-2, IL-12, or IL-15, as described herein below. One skilled in the art can determine if an anti-tumor specific immunity is induced using stand techniques in the art, which are further described in, for example, Clay, T M, et al. Clinical Cancer Research (2001); Malyguine, A, et al. J Transl Med (2004); or Macchia I, et al. BioMed Research International (2013), each of which are incorporated herein by reference in their entireties.

Interleukin-2 (IL-2) is an interleukin, a type of cytokine signaling molecule in the immune system. IL-2 regulates the activities of white blood cells (for example, leukocytes and lymphocytes) that are responsible for immunity. IL-2 is part of the body's natural response to microbial infection, and in discriminating between foreign “non-self” and “self”. It mediates its effects by binding to IL-2 receptors, which are expressed by lymphocytes. Sequences for IL-2, also known TCGF and lympokine, are known for a number of species, e.g., human IL-2 (NCBI Gene ID: 3558) polypeptide (e.g., NCBI Ref Seq NP_000577.2) and mRNA (e.g., NCBI Ref Seq NM_000586.3). IL-2 can refer to human IL-2, including naturally occurring variants, molecules, and alleles thereof. IL-2 refers to the mammalian IL-2 of, e.g., mouse, rat, rabbit, dog, cat, cow, horse, pig, and the like. The nucleic sequence of SEQ ID NO: 5 comprises the nucleic sequence which encodes IL-2.

SEQ ID NO: 5 is the nucleotide sequence encoding IL-2.

| (SEQ ID NO: 5) | |
|---|---|
| | atgta |
| 61 | caggatgcaa ctcctgtctt gcattgcact aagtcttgca cttgtcacaa acagtgcacc |
| 121 | tacttcaagt tctacaaaga aaacacagct acaactggag catttactgc tggatttaca |
| 181 | gatgattttg aatggaatta ataattacaa gaatcccaaa ctcaccagga tgctcacatt |
| 241 | taagttttac atgcccaaga aggccacaga actgaaacat cttcagtgtc tagaagaaga |
| 301 | actcaaacct ctggaggaag tgctaaattt agctcaaagc aaaaactttc acttaagacc |
| 361 | cagggactta atcagcaata tcaacgtaat agttctggaa ctaaagggat ctgaaacaac |
| 421 | attcatgtgt gaatatgctg atgagacagc aaccattgta gaatttctga acagatggat |
| 481 | taccttttgt caaagcatca tctcaacact gacttgataa |

Interleukin-12 (IL-12) is an interleukin naturally produced by dendritic cells, macrophages, neutrophils, and human B-lymphoblastoid cells (NC-37) in response to antigenic stimulation. IL-12 is involved in the differentiation of naive T cells into Th1 cells. It is known as a T cell-stimulating factor, which can stimulate the growth and function of T cells. It stimulates the production of interferon-gamma (IFN-γ) and tumor necrosis factor-alpha (TNF-α) from T cells and natural killer (NK) cells, and reduces IL-4 mediated suppression of IFN-γ. Sequences for IL-12a, also known P35, CLMF, NFSK, and KSF1, are known for a number of species, e.g., human IL-12a (NCBI Gene ID: 3592) polypeptide (e.g., NCBI Ref Seq NP_000873.2) and mRNA (e.g., NCBI Ref Seq NM 000882.3). IL-12 can refer to human IL-12, including naturally occurring variants, molecules, and alleles thereof. IL-12 refers to the mammalian IL-12 of, e.g., mouse, rat, rabbit, dog, cat, cow, horse, pig, and the like. The nucleic sequence of SEQ ID NO:6 comprises the nucleic sequence which encodes IL-12a.

SEQ ID NO: 6 is the nucleotide sequence encoding IL-12a.

| (SEQ ID NO: 6) | |
|---|---|
| | aatgtggccc cctgggtcag |
| 241 | cctcccagcc accgccctca cctgccgcgg ccacaggtct gcatccagcg gctcgccctg |
| 301 | tgtccctgca gtgccggctc agcatgtgtc cagcgcgcag cctcctcctt gtggctaccc |
| 361 | tggtcctcct ggaccacctc agtttggcca gaaacctccc cgtggccact ccagacccag |
| 421 | gaatgttccc atgccttcac cactcccaaa acctgctgag ggccgtcagc aacatgctcc |
| 481 | agaaggccag acaaactcta gaattttacc cttgcacttc tgaagagatt gatcatgaag |
| 541 | atatcacaaa agataaaacc agcacagtgg aggcctgttt accattggaa ttaaccaaga |
| 601 | atgagagttg cctaaattcc agagagacct ctttcataac taatgggagt tgcctggcct |
| 661 | ccagaaagac ctcttttatg atggccctgt gccttagtag tatttatgaa gacttgaaga |
| 721 | tgtaccaggt ggagttcaag accatgaatg caaagcttct gatggatcct aagaggcaga |
| 781 | tctttctaga tcaaaacatg ctggcagtta ttgatgagct gatgcaggcc ctgaatttca |
| 841 | acagtgagac tgtgccacaa aaatcctccc ttgaagaacc ggatttttat aaaactaaaa |
| 901 | tcaagctctg catacttctt catgctttca gaattcgggc agtgactatt gatagagtga |
| 961 | tgagctatct gaatgcttcc taa |

Interleukin-15 (IL-15) is an interleukin secreted by mononuclear phagocytes (and some other cells) following infection by virus(es). This cytokine induces cell proliferation of natural killer cells; cells of the innate immune system whose principal role is to kill virally infected cells. Sequences for IL-15 are known for a number of species, e.g., human IL-15 (NCBI Gene ID: 3600) polypeptide (e.g., NCBI Ref Seq NP_000585.4) and mRNA (e.g., NCBI Ref Seq NM_000576.1). IL-15 can refer to human IL-15, including naturally occurring variants, molecules, and alleles thereof. IL-15 refers to the mammalian IL-15 of, e.g., mouse, rat, rabbit, dog, cat, cow, horse, pig, and the like. The nucleic sequence of SEQ ID NO: 7 comprises the nucleic sequence which encodes IL-15.

SEQ ID NO: 7 is the nucleotide sequence encoding IL-15.

| (SEQ ID NO: 7) | |
|---|---|
| | atgaga atttcgaaac caca tttgag aagtatttcc atccagtgct |
| 421 | acttgtgttt acttctaaac agtcattttc taactgaagc tggcattcat gtcttcattt |
| 481 | tgggctgttt cagtgcaggg cttcctaaaa cagaagccaa ctgggtgaat gtaataagtg |
| 541 | atttgaaaaa aattgaagat cttattcaat ctatgcatat tgatgctact ttatatacgg |
| 601 | aaagtgatgt tcaccccagt tgcaaagtaa cagcaatgaa gtgctttctc ttggagttac |
| 661 | aagttatttc acttgagtcc ggagatgcaa gtattcatga tacagtagaa aatctgatca |
| 721 | tcctagcaaa caacagtttg tcttctaatg ggaatgtaac agaatctgga tgcaaagaat |
| 781 | gtgaggaact ggaggaaaaa aatattaaag aatttttgca gagttttgta catattgtcc |
| 841 | aaatgttcat caacacttct tga |

Interleukin 10 receptor (IL10R), either soluble or wild-type, has been shown to mediate the immunosuppressive signal of interleukin 10, resulting in the inhibition of the synthesis of proinflammatory cytokines. This receptor is reported to promote survival of progenitor myeloid cells through the insulin receptor substrate-2/PI 3-kinase/AKT pathway. Activation of IL10R leads to tyrosine phosphorylation of JAK1 and TYK2 kinases. Two transcript variants, one protein-coding and the other not protein-coding, have been found for this gene. Sequences for IL10R are known for a number of species, e.g., human IL10R (NCBI Gene ID: 3587) polypeptide (e.g., NCBI Ref Seq NP_001549.2) and mRNA (e.g., NCBI Ref Seq NM_001558.3). IL10R can refer to human IL10R, including naturally occurring variants, molecules, and alleles thereof. IL10R refers to the mammalian IL10R of, e.g., mouse, rat, rabbit, dog, cat, cow, horse, pig, and the like. The nucleic sequence of SEQ ID NO: 3 comprises the nucleic sequence which encodes IL10R.

SEQ ID NO: 3 is the nucleotide sequence encoding IL10R.

| (SEQ ID NO: 3) | |
|---|---|
| | atg ctgccgtgcc tcgtagtgct gctggcggcg ctcctcagcc |
| 121 | tccgtcttgg ctcagacgct catgcgacag agctgcccag ccctccgtct gtgtggtttg |
| 181 | aagcagaatt tttccaccac atcctccact ggacacccat cccaaatcag tctgaaagta |
| 241 | cctgctatga agtggcgctc ctgaggtatg gaatagagtc ctggaactcc atctccaact |
| 301 | gtagccagac cctgtcctat gaccttaccg cagtgacctt ggacctgtac cacagcaatg |
| 361 | gctaccgggc cagagtgcgg gctgtggacg gcagccggca ctccaactgg accgtcacca |
| 421 | acacccgctt ctctgtggat gaagtgactc tgacagttgg cagtgtgaac ctagagatcc |
| 481 | acaatggctt catcctcggg aagattcagc tacccaggcc caagatggcc cccgcaaatg |
| 541 | acacatatga aagcatcttc agtcacttcc gagagtatga gattgccatt cgcaaggtgc |
| 601 | cgggaaactt cacgttcaca cacaagaaag taaaacatga aaacttcagc ctcctaacct |
| 661 | ctggagaagt gggagagttc tgtgtccacg tgaaaccatc tgtcgcttcc cgaagtaaca |
| 721 | aggggatgtg gtctaaagag gagtgcatct ccctcaccag gcagtatttc accgtgacca |
| 781 | acgtcatcat cttctttgcc tttgtcctgc tgctctccgg agccctcgcc tactgcctgg |
| 841 | ccctccagct gtatgtgcgg cgccgaaaca agctacccag tgtcctgctc ttcaagaagc |
| 901 | ccagcccctt catcttcatc agccagcgtc cctccccaga gacccaagac accatccacc |
| 961 | cgcttgatga ggaggccttt ttgaaggtgt ccccagagct gaagaacttg gacctgcacg |
| 1021 | gcagcacaga cagtggcttt ggcagcacca agccatccct gcagactgaa gagccccagt |
| 1081 | tcctcctccc tgaccctcac ccccaggctg acagaacgct gggaaacagg gagccccctg |
| 1141 | tgctggggga cagctgcagt agtggcagca gcaatagcac agacagcggg atctgcctgc |
| 1201 | aggagcccag cctgagcccc agcacagggc ccacctggga gcaacaggtg gggagcaaca |
| 1261 | gcaggggcca ggatgacagt ggcattgact tagttcaaaa ctctgagggc cgggctgggg |
| 1321 | acacacaggg tggctcggcc ttgggccacc acagtccccc ggagcctgag gtgcctgggg |
| 1381 | aagaagaccc agctgctgtg gcattccagg gttacctgag gcagaccaga tgtgctgaag |
| 1441 | agaaggcaac caagacaggc tgcctggagg aagaatcgcc cttgacagat ggccttggcc |
| 1501 | ccaaattcgg gagatgcctg gttgatgagg caggcttgca tccaccagcc ctggccaagg |
| 1561 | gctatttgaa acaggatcct ctagaaatga ctctggcttc ctcaggggcc ccaacgggac |
| 1621 | agtggaacca gcccactgag gaatggtcac tcctggcctt gagcagctgc agtgacctgg |
| 1681 | gaatatctga ctggagcttt gcccatgacc ttgcccctct aggctgtgtg gcagccccag |
| 1741 | gtggtctcct gggcagcttt aactcagacc tggtcaccct gcccctcatc tctagcctgc |
| 1801 | agtcaagtga gtga |

Transforming growth factor beta receptor II (TGFBRII), either soluble or wild type form, is protein encoded by this gene forms a heteromeric complex with type II TGF-beta receptors when bound to TGF-beta, transducing the TGF-beta signal from the cell surface to the cytoplasm. Sequences for TGFBRII are known for a number of species, e.g., human TGFBRII (NCBI Gene ID: 7048) polypeptide (e.g., NCBI Ref Seq NP_001020018.1) and mRNA (e.g., NCBI Ref Seq NM_001024847.2). TGFBRII can refer to human TGFBRII, including naturally occurring variants, molecules, and alleles thereof. TGFBRII refers to the mammalian TGFBRII of, e.g., mouse, rat, rabbit, dog, cat, cow, horse, pig, and the like. The nucleic sequence of SEQ ID NO: 4 comprises the nucleic sequence which encodes TGFBRII.

SEQ ID NO: 4 is the nucleotide sequence encoding TGFBRII.

| (SEQ ID NO: 4) | |
|---|---|
| | ATGGGTCG GGGGCTGCTC AGGGGCCTGT GGCCGCTGCA |
| 421 | CATCGTCCTG TGGACGCGTA TCGCCAGCAC GATCCCACCG CACGTTCAGA AGTCGGATGT |
| 481 | GGAAATGGAG GCCCAGAAAG ATGAAATCAT CTGCCCCAGC TGTAATAGGA CTGCCCATCC |
| 541 | ACTGAGACAT ATTAATAACG ACATGATAGT CACTGACAAC AACGGTGCAG TCAAGTTTCC |
| 601 | ACAACTGTGT AAATTTTGTG ATGTGAGATT TTCCACCTGT GACAACCAGA AATCCTGCAT |
| 661 | GAGCAACTGC AGCATCACCT CCATCTGTGA GAAGCCACAG GAAGTCTGTG TGGCTGTATG |
| 721 | GAGAAAGAAT GACGAGAACA TAACACTAGA GACAGTTTGC CATGACCCCA AGCTCCCCTA |
| 781 | CCATGACTTT ATTCTGGAAG ATGCTGCTTC TCCAAAGTGC ATTATGAAGG AAAAAAAAAA |
| 841 | GCCTGGTGAG ACTTTCTTCA TGTGTTCCTG TAGCTCTGAT GAGTGCAATG ACAACATCAT |
| 901 | CTTCTCAGAA GAATATAACA CCAGCAATCC TGACTTGTTG CTAGTCATAT TTCAAGTGAC |
| 961 | AGGCATCAGC CTCCTGCCAC CACTGGGAGT TGCCATATCT GTCATCATCA TCTTCTACTG |
| 1021 | CTACCGCGTT AACCGGCAGC AGAAGCTGAG TTCAACCTGG GAAACCGGCA AGACGCGGAA |
| 1081 | GCTCATGGAG TTCAGCGAGC ACTGTGCCAT CATCCTGGAA GATGACCGCT CTGACATCAG |
| 1141 | CTCCACGTGT GCCAACAACA TCAACCACAA CACAGAGCTG CTGCCCATTG AGCTGGACAC |
| 1201 | CCTGGTGGGG AAAGGTCGCT TTGCTGAGGT CTATAAGGCC AAGCTGAAGC AGAACACTTC |
| 1261 | AGAGCAGTTT GAGACAGTGG CAGTCAAGAT CTTTCCCTAT GAGGAGTATG CCTCTTGGAA |
| 1321 | GACAGAGAAG GACATCTTCT CAGACATCAA TCTGAAGCAT GAGAACATAC TCCAGTTCCT |
| 1381 | GACGGCTGAG GAGCGGAAGA CGGAGTTGGG GAAACAATAC TGGCTGATCA CCGCCTTCCA |
| 1441 | CGCCAAGGGC AACCTACAGG AGTACCTGAC GCGGCATGTC ATCAGCTGGG AGGACCTGCG |
| 1501 | CAAGCTGGGC AGCTCCCTCG CCCGGGGGAT TGCTCACCTC CACAGTGATC ACACTCCATG |
| 1561 | TGGGAGGCCC AAGATGCCCA TCGTGCACAG GGACCTCAAG AGCTCCAATA TCCTCGTGAA |
| 1621 | GAACGACCTA ACCTGCTGCC TGTGTGACTT TGGGCTTTCC CTGCGTCTGG ACCCTACTCT |
| 1681 | GTCTGTGGAT GACCTGGCTA ACAGTGGGCA GGTGGGAACT GCAAGATACA TGGCTCCAGA |
| 1741 | AGTCCTAGAA TCCAGGATGA ATTTGGAGAA TGTTGAGTCC TTCAAGCAGA CCGATGTCTA |
| 1801 | CTCCATGGCT CTGGTGCTCT GGGAAATGAC ATCTCGCTGT AATGCAGTGG GAGAAGTAAA |
| 1861 | AGATTATGAG CCTCCATTTG GTTCCAAGGT GCGGGAGCAC CCCTGTGTCG AAAGCATGAA |
| 1921 | GGACAACGTG TTGAGAGATC GAGGGCGACC AGAAATTCCC AGCTTCTGGC TCAACCACCA |
| 1981 | GGGCATCCAG ATGGTGTGTG AGACGTTGAC TGAGTGCTGG GACCACGACC CAGAGGCCCG |
| 2041 | TCTCACAGCC CAGTGTGTGG CAGAACGCTT CAGTGAGCTG GAGCATCTGG ACAGGCTCTC |
| 2101 | GGGGAGGAGC TGCTCGGAGG AGAAGATTCC TGAAGACGGC TCCCTAAACA CTACCAAATA |
| 2161 | GCTCTTCTGG |

Antibodies or antibody reagents that bind to PD-1, or its ligand PD-L1, are described in, e.g., U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699; which are incorporated by reference herein in their entireties. In certain embodiments the PD-1 antibodies include nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD-1; AMP-224, a fusion protein of B7-DC; an antibody Fc portion; BMS-936559 (MDX-1105-01) for PD-L1 (B7-H1) blockade. Also specifically contemplated herein are agents that disrupt or block the interaction between PD-1 and PD-L1, such as a high affinity PD-L1 antagonist.

Non-limiting examples of PD-1 antibodies include: pembrolizumab (Merck); nivolumab (Bristol Meyers Squibb); pidilizumab (Medivation); and AUNP12 (Aurigene). Non-limiting examples of PD-L1 antibodies can include atezolizumab (Genentech); MPDL3280A (Roche); MEDI4736 (AstraZeneca); MSB0010718C (EMD Serono); avelumab (Merck); and durvalumab (Medimmune).

Antibodies that bind to OX40 (also known as CD134), are described in, e.g., U.S. Pat. Nos. 9,006,399, 9,738,723, 9,975,957, 9,969,810, 9,828,432; PCT Published Patent Application Nos: WO2015153513, WO2014148895, WO2017021791, WO2018002339; and U.S. application Nos: US20180273632; US20180237534; US20180230227; US20120269825; which are incorporated by reference herein in their entireties.

Antibodies that bind to CTLA-4, are described in, e.g., U.S. Pat. Nos. 9,714,290, 6,984,720, 7,605,238, 6,682,736, 7,452,535; PCT Published Patent Application No: WO2009100140; and U.S. application Nos: US20090117132A, US20030086930, US20050226875, US20090238820; which are incorporated by reference herein in their entireties. Non-limiting examples of CTLA-4 antibodies include: ipilimumab (Bristol-Myers Squibb)

Antibodies that bind to TIM3, are described in, e.g., U.S. Pat. Nos. 8,552,156, 9,605,070, 9,163,087, 8,329,660; PCT Published Patent Application No: WO2018036561, WO2017031242, WO2017178493; and U.S. application Nos: US20170306016, US20150110792, US20180057591, US20160200815; which are incorporated by reference herein in their entireties.

Antibodies that bind to TIGIT (also known as CD134), are described in, e.g., U.S. Ser. No. 10/017,572, U.S. Pat. No. 9,713,641; PCT Published Patent Application No: WO2017030823; and US application Nos: US20160355589, US20160176963, US20150322119; which are incorporated by reference herein in their entireties.

Antibodies that bind to Interleukin 10 receptor (IL10R) (e.g., soluble or wild-type) are described in, e.g., U.S. Pat. No. 7,553,932; and U.S. application Nos: US20040009939, US20030138413, US20070166307, US20090087440, and US201000028450, which are incorporated by reference herein in their entireties.

Antibodies that bind to TGFBRII (e.g., soluble or wild-type) are described in, e.g., U.S. Pat. No. 6,497,729; and U.S. application Nos: US2012114640, US20120021519, which are incorporated by reference herein in their entireties.

Another aspect provides an oncolytic Herpes Simplex Virus (HSV) comprising recombinant DNA that does not encode functional ICP0 or ICP34.5, and encodes a functional mmTGF-β2-7 M fragment sequence.

An oncolytic Herpes Simplex Virus (HSV) comprising recombinant DNA, wherein the recombinant DNA does not encode functional ICP0 and ICP34.5 genes; and encodes a functional mmTGF-β2-7 M fragment sequence.

Another aspect provides an oncolytic HSV comprising recombinant DNA that does not encode functional ICP0, and encodes a functional mmTGF-β2-7 M fragment sequence.

Another aspect provides an oncolytic HSV encoding a functional mmTGF-β2-7 M fragment sequence.

Yet another aspect provides a recombinant virus encoding a functional mmTGF-β2-7 M fragment sequence.

In one embodiment, the any of the oncolytic HSVs described herein further encodes fusogenic activity.

One aspect of the invention described herein provides a composition comprising any of the oncolytic HSV described herein. In one embodiment, the composition is a pharmaceutical composition. As used herein, the term “pharmaceutical composition” refers to the active agent in combination with a pharmaceutically acceptable carrier e.g. a carrier commonly used in the pharmaceutical industry.

In one embodiment, the composition further comprises at least one pharmaceutically acceptable carrier. Pharmaceutically acceptable carriers are well known in the art and include aqueous solutions such as physiologically buffered saline or other solvents or vehicles such as glycols, glycerol, vegetable oils (e.g., olive oil) or injectable organic esters. A pharmaceutically acceptable carrier can be used to administer the compositions of the invention to a cell in vitro or to a subject in vivo. A pharmaceutically acceptable carrier can contain a physiologically acceptable compound that acts, for example, to stabilize the composition or to increase the absorption of the agent. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. One skilled in the art would know that the choice of a pharmaceutically acceptable carrier, including a physiologically acceptable compound, depends, for example, on the route of administration of the oncolytic HSV.

### Hybrid Nucleic Acids

Another aspect provided herein is a hybrid nucleic acid sequence containing a sequence of a therapeutic antibody and a dominate negative TGFβ, wherein dominate negative TGFβ is fused to a Fc domain of the therapeutic antibody.

Another aspect provided herein is a hybrid nucleic acid sequence containing a sequence of a therapeutic antibody and a mmTGF-β2-7 M fragment, wherein mmTGF-β2-7 M is fused to a Fc domain of the therapeutic antibody.

In one embodiment, the therapeutic antibody sequence is a sequence of an immunotherapeutic antibody. For example, the therapeutic antibody sequence is a sequence can be selected from the list consisting of an anti-PD-1 antibody, an anti-PD-L1 antibody, an anti-Tim3 antibody, an anti-anti-CTLA4 antibody, and anti-TDM-1 antibody, and an anti-TIGIT antibody. Such therapeutic antibodies are described herein above.

Also provide herein are polypeptides encoded by any of the hybrid nucleic acids described herein.

Further provided herein are vectors expressing any of the hybrid nucleic acids described herein.

### Chimeric Antigen Receptors

The technology described herein provides improved CARS for use in treatment of cancer. The following discusses CARs and the various improvements.

The terms “chimeric antigen receptor” or “CAR” or “CARs” as used herein refer to engineered T cell receptors, which graft a ligand or antigen specificity onto T cells (for example naïve T cells, central memory T cells, effector memory T cells or combinations thereof). CARs are also known as artificial T-cell receptors, chimeric T-cell receptors or chimeric immunoreceptors.

A CAR places a chimeric extracellular target-binding domain that specifically binds a target, e.g., a polypeptide, expressed on the surface of a cell to be targeted for a T cell response onto a construct including a transmembrane domain and intracellular domain(s) of a T cell receptor molecule. In one embodiment, the chimeric extracellular target-binding domain comprises the antigen-binding domain(s) of an antibody that specifically binds an antigen expressed on a cell to be targeted for a T cell response. The properties of the intracellular signaling domain(s) of the CAR can vary as known in the art and as disclosed herein, but the chimeric target/antigen-binding domains(s) render the receptor sensitive to signaling activation when the chimeric target/antigen binding domain binds the target/antigen on the surface of a targeted cell.

With respect to intracellular signaling domains, so-called “first-generation” CARs include those that solely provide CD3zeta (CD3ζ) signals upon antigen binding. So-called “second-generation” CARs include those that provide both co-stimulation (e.g., CD28 or CD 137) and activation (CD3ζ) domains, and so-called “third-generation” CARs include those that provide multiple costimulatory (e.g., CD28 and CD 137) domains and activation domains (e.g., CD3ζ). In various embodiments, the CAR is selected to have high affinity or avidity for the target/antigen—for example, antibody-derived target or antigen binding domains will generally have higher affinity and/or avidity for the target antigen than would a naturally-occurring T cell receptor. This property, combined with the high specificity one can select for an antibody provides highly specific T cell targeting by CAR T cells.

As used herein, a “CAR T cell” or “CAR-T” refers to a T cell which expresses a CAR. When expressed in a T cell, CARs have the ability to redirect T-cell specificity and reactivity toward a selected target in a non-MHC-restricted manner, exploiting the antigen-binding properties of monoclonal antibodies. The non-MHC-restricted antigen recognition gives T-cells expressing CARS the ability to recognize an antigen independent of antigen processing, thus bypassing a major mechanism of tumor escape.

As used herein, the term “extracellular target binding domain” refers to a polypeptide found on the outside of the cell which is sufficient to facilitate binding to a target. The extracellular target binding domain will specifically bind to its binding partner, i.e. the target. As non-limiting examples, the extracellular target-binding domain can include a sequence encoding a dominate negative peptide, an antigen-binding domain of an antibody, or a ligand, which recognizes and binds with a cognate binding partner (for example, TGFβ) protein.

In one embodiment, the CAR is a bi-specific CAR. For example, the CAR comprises in its extracellular domain a dominate negative TGFβ sequence, e.g., mmTGF-f32-7 M fragment; and a sequence of a therapeutic antibody, for example, an anti-PD1 antibody, an anti-CTLA4 antibody, or an anti-TIM3 antibody.

### Transmembrane Domain

Each CAR as described herein necessarily includes a transmembrane domain that joins the extracellular target-binding domain to the intracellular signaling domain.

As used herein, “transmembrane domain” (TM domain) refers to the generally hydrophobic region of the CAR which crosses the plasma membrane of a cell. The TM domain can be the transmembrane region or fragment thereof of a transmembrane protein (for example a Type I transmembrane protein or other transmembrane protein), an artificial hydrophobic sequence, or a combination thereof. While specific examples are provided herein and used in the Examples, other transmembrane domains will be apparent to those of skill in the art and can be used in connection with alternate embodiments of the technology. A selected transmembrane region or fragment thereof would preferably not interfere with the intended function of the CAR. As used in relation to a transmembrane domain of a protein or polypeptide, “fragment thereof” refers to a portion of a transmembrane domain that is sufficient to anchor or attach a protein to a cell surface.

In one embodiment, the transmembrane domain or fragment thereof of the CAR described herein comprises a transmembrane domain selected from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1(CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

In one exemplary embodiment, a CAR's transmembrane domain or fragment thereof is derived from or comprises the transmembrane domain of CD8. CD8 is an antigen preferentially found on the cell surface of cytotoxic T lymphocytes. CD8 mediates cell-cell interactions within the immune system, and acts as a T cell coreceptor. CD8 consists of an alpha (CD8a) and beta (CD8β) chain. CD8a sequences are known for a number of species, e.g., human CD8a, (NCBI Gene ID: 925) polypeptide (e.g., NCBI Ref Seq NP_001139345.1) and mRNA (e.g., NCBI Ref Seq NM_000002.12). CD8 can refer to human CD8, including naturally occurring variants, molecules, and alleles thereof. In some embodiments of any of the aspects, e.g., in veterinary applications, CD8 can refer to the CD8 of, e.g., dog, cat, cow, horse, pig, and the like. Homologs and/or orthologs of human CD8 are readily identified for such species by one of skill in the art, e.g., using the NCBI ortholog search function or searching available sequence data for a given species for sequence similar to a reference CD8 sequence.

### Co-Stimulatory Domain

A CAR described herein can comprise an intracellular domain of a co-stimulatory molecule, or co-stimulatory domain. As used herein, the term “co-stimulatory domain” refers to an intracellular signaling domain of a co-stimulatory molecule. Co-stimulatory molecules are cell surface molecules other than antigen receptors or Fc receptors that provide a second signal required for efficient activation and function of T lymphocytes upon binding to antigen. Illustrative examples of such co-stimulatory molecules include CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70.

In one exemplary embodiment, the intracellular domain is the intracellular domain of 4-1BB. 4-1BBL is a type 2 transmembrane glycoprotein belonging to the TNF superfamily. 4-1BBL is expressed on activated T lymphocytes. 4-1BBL sequences are known for a number of species, e.g., human 4-1BBL, also known as TNFSF9 (NCBI Gene ID: 8744) polypeptide (e.g., NCBI Ref Seq NP_003802.1) and mRNA (e.g., NCBI Ref Seq NM_003811.3). 4-1BBL can refer to human 4-1BBL, including naturally occurring variants, molecules, and alleles thereof. In some embodiments of any of the aspects, e.g., in veterinary applications, 4-1BBL can refer to the 4-1BBL of, e.g., dog, cat, cow, horse, pig, and the like. Homologs and/or orthologs of human 4-1BBL are readily identified for such species by one of skill in the art, e.g., using the NCBI ortholog search function or searching available sequence data for a given species for sequence similar to a reference 4-1BBL sequence.

### Intracellular Signaling Domain

CARs as described herein can comprise an intracellular signaling domain. An “intracellular signaling domain,” refers to the part of a CAR polypeptide that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited following antigen binding to the extracellular CAR domain.

CD3 is a T cell co-receptor that facilitates T lymphocytes activation when simultaneously engaged with the appropriate co-stimulation (e.g., binding of a co-stimulatory molecule). A CD3 complex consists of 4 distinct chains; mammal CD3 consists of a CD3γchain, a CD3δ chain, and two CD3ε chains. These chains associate with a molecule known as the T cell receptor (TCR) and the CD3ζ to generate an activation signal in T lymphocytes. A complete TCR complex comprises a TCR, CD3ζ, and the complete CD3 complex.

In some embodiments of any aspect, a CAR polypeptide described herein comprises an intracellular signaling domain that comprises an Immunoreceptor Tyrosine-based Activation Motif or ITAM from CD3 zeta (CD3ζ). In some embodiments of any aspect, the ITAM comprises three motifs of ITAM of CD3ζ (ITAM3).

ITAMs are known as a primary signaling domains which regulate primary activation of the TCR complex either in a stimulatory way, or in an inhibitory way. Primary signaling domains that act in a stimulatory manner may contain signaling motifs which are known as immunoreceptor tyrosine-based activation motifs or ITAMs. Non-limiting examples of ITAM-containing intracellular signaling domains that are of particular use in the technology include those derived from TCRζ, FcRγ, FcRβ, CD3γ, CD3□, CD3δ, CD3ε, CD3ζ, CD22, CD79a, CD79b, and CD66d.

In one embodiment, the CAR further comprises a linker domain. As used herein “linker domain” refers to an oligo- or polypeptide region from about 2 to 100 amino acids in length, which links together any of the domains/regions of the CAR as described herein. In some embodiment, linkers can include or be composed of flexible residues such as glycine and serine so that the adjacent protein domains are free to move relative to one another. Longer linkers may be used when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof. In one embodiment, the linker region is T2A derived from Thosea asigna virus. Non-limiting examples of linkers that can be used in this technology include P2A and F2A.

A more detailed description of CARs and CAR T cells can be found in Maus et al. Blood 2014 123:2624-35; Reardon et al. Neuro-Oncology 2014 16:1441-1458; Hoyos et al. Haematologica 2012 97:1622; Byrd et al. J Clin Oncol 2014 32:3039-47; Maher et al. Cancer Res 2009 69:4559-4562; and Tamada et al. Clin Cancer Res 2012 18:6436-6445; each of which is incorporated by reference herein in its entirety.

Another aspect provided herein is nucleic acid encoding any of the CAR polypeptides described herein.

### Cells

Provided herein is a cell or populations thereof comprising any of the oncolytic or recombinant viruses described herein.

Provided herein is a cell or populations thereof comprising any of the hybrid nucleic acids, polypeptides encoding a hybrid nucleic acid, or a vector expressing the hybrid nucleic acids or polypeptides encoding a hybrid nucleic acid.

Further provided herein is a cell or population thereof comprising any of the CAR polypeptides, or any of the nucleic acids encoding a CAR polypeptide described herein.

In one embodiment, the cell is a mammalian cells. In one embodiment, the cell is a human cell. In one embodiment, the cell is a non-human mammalian cell.

In one embodiment, the cell is a T cell. In one embodiment, the cell is a CAR T cell.

In one embodiment, the cell is an immune cell. As used herein, “immune cell” refers to a cell that plays a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes. In some embodiments, the cell is a T cell; a NK cell; a NKT cell; lymphocytes, such as B cells and T cells; and myeloid cells, such as monocytes, macrophages, eosinophils, mast cells, basophils, and granulocytes.

In one embodiment, the cell is obtained from an individual having or diagnosed as having cancer.

In one embodiment, the cell is a CAR T cell.

In one embodiment, the cell is a bi-specific CAR T cell, meaning is comprising more than one CAR polypeptide. For example, the CAR T cells comprises a CAR polypeptide having an extracellular domain comprising a dominate negative TGFβ sequence, e.g., mmTGF-β2-7 M fragment, and second CAR polypeptide comprising an extracellular domain comprising a sequence of a therapeutic antibody, for example, an anti-PD1 antibody, an anti-CTLA4 antibody, or an anti-TIM3 antibody.

In certain embodiment, the cell has high levels of a dominant negative TGFβ, e.g., mmTGF-β2-7 M. Expression levels of a dominant negative TGFβ can be determined by one skilled in the art, e.g., via western blotting or PCR-based assays to assess the protein or mRNA levels of the dominant negative TGFβB, respectively.

### Methods of Treatment

The oncolytic viruses, hybrid nucleic acids, and CAR T cells described herein or composition thereof, can be administered to a subject having cancer. In certain embodiments, where appropriate, an agent that regulates the tet operator of the oncolytic virus is further administered with the oncolytic viruses described herein or composition thereof. Exemplary agents include, but are not limited to, doxycycline or tetracycline.

One aspect provides a method of treating cancer, the method comprising engineering a T cell to comprise any of the CAR polypeptides or nucleic acids encoding the CAR polypeptide described herein on the T cell surface; and administering the engineered T cell to the subject

In one embodiment, the cancer is a solid tumor. The solid tumor can be malignant or benign. In one embodiment, the subject is diagnosed or has been diagnosed with having a carcinoma, a melanoma, a sarcoma, a germ cell tumor, and a blastoma. Exemplary cancers include, but are in no way limited to, non-small-cell lung cancer, bladder cancer, breast cancer, brain cancer, colon cancer, prostate cancer, liver cancer, lung cancer, ovarian cancer, skin cancer, head and neck cancer, kidney cancer, and pancreatic cancer. In one embodiment, the cancer is metastatic. These types of cancers are known in the art and can be diagnosed by a skilled clinician using standard techniques known in the art, for example blood analysis, blood cell count analysis, tissue biopsy, non-invasive imaging, and/or review of family history.

In cases where tumors are readily accessible, e.g., tumors of the skin, mouth or which are accessible as the result of surgery, virus can be applied topically. In other cases, it can be administered by injection or infusion. The agent that regulates the tet operator and tetR interaction, for example doxycycline or tetracycline, used prior to infection or at a time of infection can also be administered in this way or it can be administered systemically, for example, orally.

Although certain routes of administration are provided in the foregoing description, according to the invention, any suitable route of administration of the vectors may be adapted, and therefore the routes of administration described above are not intended to be limiting. Routes of administration may include, but are not limited to, intravenous, regional artery infusion, oral, buccal, intranasal, inhalation, topical application to a mucosal membrane or injection, including intratumoral, intradermal, intrathecal, intracisternal, intralesional or any other type of injection. Administration can be effected continuously or intermittently and will vary with the subject and the condition to be treated. One of skill in the art would readily appreciate that the various routes of administration described herein would allow for the inventive vectors or compositions to be delivered on, in, or near the tumor or targeted cancer cells. One of skill in the art would also readily appreciate that various routes of administration described herein will allow for the vectors and compositions described herein to be delivered to a region in the vicinity of the tumor or individual cells to be treated. “In the vicinity” can include any tissue or bodily fluid in the subject that is in sufficiently close proximity to the tumor or individual cancer cells such that at least a portion of the vectors or compositions administered to the subject reach their intended targets and exert their therapeutic effects.

Prior to administration, the oncolytic viruses can be suspended in any pharmaceutically acceptable solution including sterile isotonic saline, water, phosphate buffered saline, 1,2-propylene glycol, polyglycols mixed with water, Ringer's solution, etc. The exact number of viruses to be administered is not crucial to the invention but should be an “effective amount,” i.e., an amount sufficient to cause cell lysis extensive enough to generate an immune response to released tumor antigens. Since virus is replicated in the cells after infection, the number initially administered will increase rapidly with time. Thus, widely different amounts of initially administered virus can give the same result by varying the time that they are allowed to replicate, i.e., the time during which cells are exposed to tetracycline. In general, it is expected that the number of viruses (PFU) initially administered will be between 1×10⁶ and 1×10¹⁰.

Tetracycline or doxycycline will be administered either locally or systemically to induce viral replication at a time of infection or 1-72 h prior to infection. The amount of tetracycline or doxycycline to be administered will depend upon the route of delivery. In vitro, 1 μg/ml of tetracycline is more than sufficient to allow viral replication in infected cells. Thus, when delivered locally, a solution containing anywhere from 0.1 μg/ml to 100 μg/ml may be administered. However, much higher doses of tetracycline or doxycycline (e.g., 1-5 mg/ml) can be employed if desired. The total amount given locally at a single time will depend on the size of the tumor or tumors undergoing treatment but in general, it is expected that between 0.5 and 200 ml of tetracycline or doxycycline solution would be used at a time. When given systemically, higher doses of tetracycline or doxycycline will be given but it is expected that the total amount needed will be significantly less than that typically used to treat bacterial infections (for example, with doxycycline, usually 1-2 grams per day in adults divided into 2-4 equal doses and, in children, 2.2-4.4 mg per kilogram of body weight, which can be divided into at least 2 doses, per day). It is expected that 5-100 mg per day should be effective in most cases. Dosing for tetracycline and doxycycline are well known in the art and can best be determined by a skilled clinician for a given patient.

In some embodiments, the pharmaceutical composition comprising CAR T cells as described herein can be a parenteral dose form. Since administration of parenteral dosage forms typically bypasses the patient's natural defenses against contaminants, the components apart from the CAR T cells themselves are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. Any of these can be added to the CAR T cells preparation prior to administration.

Suitable vehicles that can be used to provide parenteral dosage forms of CAR T cells as disclosed within are well known to those skilled in the art. Examples include, without limitation: saline solution; glucose solution; aqueous vehicles including but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

In some embodiments, the CAR T cells described herein are administered as a monotherapy, i.e., another treatment for the condition is not concurrently administered to the subject.

A pharmaceutical composition comprising the T cells described herein can generally be administered at a dosage of 10⁴ to 10⁹ cells/kg body weight, in some instances 10⁵ to 10⁶ cells/kg body weight, including all integer values within those ranges. If necessary, T cell compositions can also be administered multiple times at these dosages. The cells can be administered by using infusion techniques that are commonly known in immunotherapy (see, e.g., Rosenberg et al., New Eng. J. of Med. 319:1676, 1988).

In certain aspects, it may be desired to administer CAR T cells to a subject and then subsequently redraw blood (or have an apheresis performed), activate T cells therefrom as described herein, and reinfuse the patient with these activated and expanded T cells. This process can be carried out multiple times every few weeks. In certain aspects, T cells can be activated from blood draws of from 10 cc to 400 cc. In certain aspects, T cells are activated from blood draws of 20 cc, 30 cc, 40 cc, 50 cc, 60 cc, 70 cc, 80 cc, 90 cc, or 100 cc.

Modes of administration can include, for example intravenous (i.v.) injection or infusion. The compositions described herein can be administered to a patient transarterially, intratumorally, intranodally, or intramedullary. In some embodiments, the compositions of T cells may be injected directly into a tumor, lymph node, or site of infection. In one embodiment, the compositions described herein are administered into a body cavity or body fluid (e.g., ascites, pleural fluid, peritoneal fluid, or cerebrospinal fluid).

As used herein, the term “therapeutically effective amount” is intended to mean the amount of vector which exerts oncolytic activity, causing attenuation or inhibition of tumor cell proliferation, leading to tumor regression. An effective amount will vary, depending upon the pathology or condition to be treated, by the patient and his or her status, and other factors well known to those of skill in the art. Effective amounts are easily determined by those of skill in the art. In some embodiments a therapeutic range is from 10³ to 10¹² plaque forming units introduced once. In some embodiments a therapeutic dose in the aforementioned therapeutic range is administered at an interval from every day to every month via the intratumoral, intrathecal, convection-enhanced, intravenous or intra-arterial route.

### Combinational Therapy

The oncolytic viruses and CAR T cells described herein can be used in combination with other known agents and therapies. In one embodiment, the subject is further administered an anti-cancer therapy. Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The oncolytic viruses or CAR T cells described herein and the at least one additional therapeutic agent can be administered simultaneously, in the same or in separate compositions, or sequentially. For sequential administration, the oncolytic viruses or CAR T cells described herein can be administered first, and the additional agent can be administered second, or the order of administration can be reversed. The oncolytic viruses or CAR T cells and/or other therapeutic agents, procedures or modalities can be administered during periods of active disorder, or during a period of remission or less active disease. The oncolytic viruses or CAR T cells can be administered before another treatment, concurrently with the treatment, post-treatment, or during remission of the disorder.

When administered in combination, the oncolytic viruses or CAR T cells and the additional agent (e.g., second or third agent), or all, can be administered in an amount or dose that is higher, lower or the same as the amount or dosage of each agent used individually, e.g., as a monotherapy. In certain embodiments, the administered amount or dosage of the oncolytic viruses or CAR T cells, the additional agent (e.g., second or third agent), or all, is lower (e.g., at least 20%, at least 30%, at least 40%, or at least 50%) than the amount or dosage of each agent used individually. In other embodiments, the amount or dosage of the oncolytic viruses or CAR T cells, the additional agent (e.g., second or third agent), or all, that results in a desired effect (e.g., treatment of cancer) is lower (e.g., at least 20%, at least 30%, at least 40%, or at least 50% lower) than the amount or dosage of each agent individually required to achieve the same therapeutic effect. In further embodiments, the oncolytic viruses or CAR T cells described herein can be used in a treatment regimen in combination with surgery, chemotherapy, radiation, an mTOR pathway inhibitor, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, rapamycin, mycophenolic acid, steroids, FR901228, cytokines, or a peptide vaccine, such as that described in Izumoto et al. 2008 J Neurosurg 108:963-971.

In one embodiment, the oncolytic viruses or CAR T cells described herein can be used in combination with a checkpoint inhibitor. Exemplary checkpoint inhibitors include anti-PD-1 inhibitors (Nivolumab, MK-3475, Pembrolizumas, Pidilizumab, AMP-224, AMP-514), anti-CTLA4 inhibitors (Ipilimumab and Tremelimumab), anti-PDL1 inhibitors (Atezolizumab, Avelumab, MSB0010718C, MEDI4736, and MPDL3280A), and anti-TIM3 inhibitors.

In one embodiment, the oncolytic viruses or CAR T cells described herein can be used in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzumab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitabine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®). Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil Nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®). Exemplary mTOR inhibitors include, e.g., temsirolimus; ridaforolimus (formally known as deferolimus, (1R,2R,45)-4-[(2R)-2 [(1R,95,125,15R,16E,18R,19R,21R,235,24E,26E,28Z,305,325,35R)-1,18-dihydroxy-19,30-dimethoxy-15,17,21,23,29,35-hexamethyl-2,3,10,14,20-pentaoxo-11,36-dioxa-4-azatricyclo[30.3.1.04′9]hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RADOO1); rapamycin (AY22989, Sirolimus®); simapimod (CAS 164301-51-3); emsirolimus, (5-{2,4-Bis[(35)-3-methylmorpholin-4-yl]pyrido[2,3-(i]pyrimidin-7-yl}-2-methoxyphenyl)methanol (AZD8055); 2-Amino-8-[iraw5,-4-(2-hydroxyethoxy)cyclohexyl]-6-(6-methoxy-3-pyridinyl)-4-methyl-pyrido[2,3-JJpyrimidin-7(8H)-one (PF04691502, CAS 1013101-36-4); and N2-[1,4-dioxo-4-[[4-(4-oxo-8-phenyl-4H-1-benzopyran-2-yl)morpholinium-4-yl]methoxy]butyl]-L-arginylglycyl-L-a-aspartylL-serine-(SEQ ID NO: 29), inner salt (SF1126, CAS 936487-67-1), and XL765. Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics). Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin. Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®). Exemplary proteosome inhibitors include bortezomib (Velcade®); carfilzomib (PX-171-007, (5)-4-Methyl-N-((5)-1-(((5)-4-methyl-1-((R)-2-methyloxiran-2-yl)-1-oxopentan-2-yl)amino)-1-oxo-3-phenylpropan-2-yl)-2-((5)-2-(2-morpholinoacetamido)-4-phenylbutanamido)-pentanamide); marizomib (NPT0052); ixazomib citrate (MLN-9708); delanzomib (CEP-18770); and O-Methyl-N-[(2-methyl-5-thiazolyl)carbonyl]-L-seryl-O-methyl-N-[(1lS′)-2-[(2R)-2-methyl-2-oxiranyl]-2-oxo-1-(phenylmethyl)ethyl]-L-serinamide (ONX-0912).

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

In an embodiment, oncolytic viruses or CAR T cells described herein are administered to a subject in combination with a molecule that decreases the level and/or activity of a molecule targeting GITR and/or modulating GITR functions, a molecule that decreases the Treg cell population, an mTOR inhibitor, a GITR agonist, a kinase inhibitor, a non-receptor tyrosine kinase inhibitor, a CDK4 inhibitor, and/or a BTK inhibitor.

### Efficacy

The efficacy of oncolytic viruses or CAR T cells in, e.g. the treatment of a condition described herein, or to induce a response as described herein (e.g. a reduction in cancer cells, shrinkage of tumor size) can be determined by the skilled clinician. However, a treatment is considered “effective treatment,” as the term is used herein, if one or more of the signs or symptoms of a condition described herein is altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Treatment according to the methods described herein can reduce levels of a marker or symptom of a condition, e.g. by at least 10%, at least 15%, at least 20%, at least 25%, at least 30%, at least 40%, at least 50%, at least 60%, at least 70%, at least 80% or at least 90% or more.

Efficacy can also be measured by a failure of an individual to worsen as assessed by hospitalization, or need for medical interventions (i.e., progression of the disease is halted). Methods of measuring these indicators are known to those of skill in the art and/or are described herein.

Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy of a given approach can be assessed in animal models of a condition described herein, for example treatment of ALL. When using an experimental animal model, efficacy of treatment is evidenced when a statistically significant change in a marker is observed.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior technology or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

The invention provided herein can further be described in the following numbered paragraphs.
1. An oncolytic Herpes Simplex Virus (HSV) comprising recombinant DNA, wherein the recombinant DNA comprises:
a) a gene comprising a 5′ untranslated region and a HSV-1, or HSV-2, VP5 gene that is operably linked to an VP5 promoter comprising a TATA element;
b) a tetracycline operator sequence positioned between 6 and 24 nucleotides 3′ to said TATA element, wherein the VP5 gene lies 3′ to said tetracycline operator sequence;
c) a gene sequence encoding tetracycline repressor operably linked to an HSV immediate-early promoter, wherein the gene sequence is located at the ICP0 locus;
d) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant;
e) a gene sequence encoding a functional ICP34.5 protein; and
f) a gene sequence operably linked to a modified HSV promoter, wherein the gene is located in an intergenic region of UL26 and UL27 genes, wherein said oncolytic HSV does not encode functional ICP0 and does not contain a ribozyme sequence located in said 5′ untranslated region of VP5.
2. An oncolytic Herpes Simplex Virus (HSV) comprising recombinant DNA, wherein the recombinant DNA comprises:
a) a gene comprising a 5′ untranslated region and a HSV-1, or HSV-2, VP5 gene that is operably linked to an VP5 promoter comprising a TATA element;
b) a tetracycline operator sequence positioned between 6 and 24 nucleotides 3′ to said TATA element, wherein the VP5 gene lies 3′ to said tetracycline operator sequence;
c) a gene sequence encoding tetracycline repressor operably linked to an HSV immediate-early promoter, wherein the gene sequence is located at the ICP0 locus;
d) a variant gene that increases syncytium formation as compared to wild type, wherein the HSV-1, or HSV-2, variant gene is selected from the group consisting of a glycoprotein K (gK) variant; a glycoprotein B (gB) variant; a UL24 variant; and UL20 gene variant;
e) a gene sequence encoding a functional ICP34.5 protein; and
f) a gene sequence operably linked to a modified HSV promoter, wherein the gene is located in an intergenic region of UL21 and UL22 genes, wherein said oncolytic HSV does not encode functional ICP0 and does not contain a ribozyme sequence located in said 5′ untranslated region of VP5.
3. An oncolytic Herpes Simplex Virus (HSV) comprising recombinant DNA, wherein the recombinant DNA comprises:
a) a gene comprising a 5′ untranslated region and a HSV-1, or HSV-2, VP5 gene that is operably linked to an VP5 promoter comprising a TATA element;
b) a tetracycline operator sequence positioned between 6 and 24 nucleotides 3′ to said TATA element, wherein the VP5 gene lies 3′ to said tetracycline operator sequence;
c) a gene sequence encoding tetracycline repressor operably linked to an HSV immediate-early promoter, wherein the gene sequence is located at the ICP0 locus;
d) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant;
e) a gene sequence encoding a functional ICP34.5 protein; and
f) a gene sequence operably linked to a modified HSV promoter, wherein the gene is located in an intergenic region of UL26, UL27, UL21 and UL 21 genes, wherein said oncolytic HSV does not encode functional ICP0 and does not contain a ribozyme sequence located in said 5′ untranslated region of VP5.
4. The oncolytic HSV of any preceding paragraph, wherein the gene sequence of (f) is a LacZ gene sequence.
5. The oncolytic HSV of any preceding paragraph, wherein the gene sequence of (f) is a dominant-negative TGF-β mutant sequence.
6. The oncolytic HSV of any preceding paragraph, wherein the dominant-negative TGF-β mutant sequence is a mmTGF-β2-7 M fragment sequence.
7. The oncolytic HSV of any preceding paragraph, wherein the promoter of (f) is a modified HSV immediate-early promoter, an HCMV immediate-early promoter, or a human elongation alpha promoter.
8. The oncolytic HSV of any preceding paragraph, wherein the variant gene is a gK variant gene that encodes an amino acid substitution selected from the group consisting of: an Ala to Thr amino acid substitution corresponding to amino acid 40 of SEQ ID NO: 2; an Ala to “x” amino acid substitution corresponding to amino acid 40 of SEQ ID NO: 2, wherein “x” is any amino acid; an Asp to Asn amino acid substitution corresponding to amino acid 99 of SEQ ID NO: 2; a Len to Pro amino acid substitution corresponding to amino acid 304 of SEQ ID NO: 2; and an Arg to Len amino acid substitution corresponding to amino acid 310 of SEQ ID NO: 2.
9. The oncolytic HSV of any preceding paragraph, wherein the tetracycline operator sequence comprises two Op2 repressor binding sites.
10. The oncolytic HSV of any preceding paragraph, wherein the VP5 promoter is an HSV-1 or HSV-2 VP5 promoter.
11. The oncolytic HSV of any preceding paragraph, wherein the immediate-early promoter is an HSV-1 or HSV-2 immediate-early promoter.
12. The oncolytic HSV of any preceding paragraph, wherein the HSV immediate-early promoter is selected from the group consisting of: ICP0 promoter, ICP4 promoter and ICP27 promoter.
13. The oncolytic HSV of any preceding paragraph, wherein the recombinant DNA is part of the HSV-1 genome.
14. The oncolytic HSV of any preceding paragraph, wherein the recombinant DNA is part of the HSV-2 genome.
15. The oncolytic HSV of any preceding paragraph, further comprising a pharmaceutically acceptable carrier.
16. The oncolytic HSV of any preceding paragraph, further encoding at least one polypeptide that can increase the efficacy of the oncolytic HSV to induce an anti-tumor-specific immunity.
17. The oncolytic HSV of any preceding paragraph, wherein the at least one polypeptide encodes a product selected from the group consisting of interleukin 2 (IL2), interleukin 12 (IL12), interleukin 15 (IL15), an anti-PD-1 antibody or antibody reagent, an anti-PD-L1 antibody or antibody reagent, an anti-OX40 antibody or antibody reagent, a CTLA-4 antibody or antibody reagent, a TIM-3 antibody or antibody reagent, a TIGIT antibody or antibody reagent, a soluble interleukin 10 receptor (IL10R), a fusion polypeptide between a soluble IL10R and IgG-Fc domain, a soluble TGFβ type II receptor (TGFBRII), a fusion polypeptide between a soluble TGFBRII and IgG-Fc domain, an anti-IL10R antibody or antibody reagent, an anti-IL10 antibody or antibody reagent, an anti-TGFBRII antibody or antibody reagent, and an anti-TGFBRII antibody or antibody reagent.
18. The oncolytic HSV of any preceding paragraph, wherein the oncolytic HSV the further encodes fusogenic activity.
19. An oncolytic Herpes Simplex Virus (HSV) comprising recombinant DNA, wherein the recombinant DNA comprises:
a) a gene comprising a 5′ untranslated region and a HSV-1, or HSV-2, VP5 gene that is operably linked to an VP5 promoter comprising a TATA element;
b) a tetracycline operator sequence positioned between 6 and 24 nucleotides 3′ to said TATA element, wherein the VP5 gene lies 3′ to said tetracycline operator sequence;
c) a gene sequence encoding tetracycline repressor operably linked to an HSV immediate-early promoter, wherein the gene sequence is located at the ICP0 locus;
d) a variant gene that increases syncytium formation as compared to wild type, wherein the HSV-1, or HSV-2, variant gene is selected from the group consisting of a glycoprotein K (gK) variant; a glycoprotein B (gB) variant; a UL24 variant; and UL20 gene variant;
e) a gene sequence encoding a functional ICP34.5 protein; and
f) a dominant-negative TGF-β mutant sequence operably linked to a modified HSV-2 immediate-early promoter, wherein the gene is located in an intergenic region of UL26 and UL27 genes.
wherein said oncolytic HSV does not encode functional ICP0 and does not contain a ribozyme sequence located in said 5′ untranslated region of VP5.
20. An oncolytic Herpes Simplex Virus (HSV) comprising recombinant DNA, wherein the recombinant DNA comprises:
a) a gene comprising a 5′ untranslated region and a HSV-1, or HSV-2, VP5 gene that is operably linked to an VP5 promoter comprising a TATA element;
b) a tetracycline operator sequence positioned between 6 and 24 nucleotides 3′ to said TATA element, wherein the VP5 gene lies 3′ to said tetracycline operator sequence;
c) a gene sequence encoding tetracycline repressor operably linked to an HSV immediate-early promoter, wherein the gene sequence is located at the ICP0 locus;
d) a variant gene that increases syncytium formation as compared to wild type, wherein the HSV-1, or HSV-2, variant gene is selected from the group consisting of a glycoprotein K (gK) variant; a glycoprotein B (gB) variant; a UL24 variant; and UL20 gene variant;
e) a gene sequence encoding a functional ICP34.5 protein; and
f) a dominant-negative TGF-β mutant sequence operably linked to a modified HSV-2 immediate-early promoter, wherein the gene is located in an intergenic region of UL21 and UL22 genes.
wherein said oncolytic HSV does not encode functional ICP0 and does not contain a ribozyme sequence located in said 5′ untranslated region of VP5.
21. An oncolytic Herpes Simplex Virus (HSV) comprising recombinant DNA, wherein the recombinant DNA comprises:
a) a gene comprising a 5′ untranslated region and a HSV-1, or HSV-2, VP5 gene that is operably linked to an VP5 promoter comprising a TATA element;
b) a tetracycline operator sequence positioned between 6 and 24 nucleotides 3′ to said TATA element, wherein the VP5 gene lies 3′ to said tetracycline operator sequence;
c) a gene sequence encoding tetracycline repressor operably linked to an HSV immediate-early promoter, wherein the gene sequence is located at the ICP0 locus;
d) a variant gene that increases syncytium formation as compared to wild type, wherein the HSV-1, or HSV-2, variant gene is selected from the group consisting of a glycoprotein K (gK) variant; a glycoprotein B (gB) variant; a UL24 variant; and UL20 gene variant;
e) a gene sequence encoding a functional ICP34.5 protein; and
f) a dominant-negative TGF-β mutant sequence operably linked to a modified HSV-2 immediate-early promoter, wherein the gene is located in an intergenic region of UL 21, UL22, UL26 and UL27 genes.
wherein said oncolytic HSV does not encode functional ICP0 and does not contain a ribozyme sequence located in said 5′ untranslated region of VP5.
22. The oncolytic HSV of any preceding paragraph, wherein the dominant-negative TGF-β mutant sequence is a mmTGF-β2-7 M fragment sequence.
23. The oncolytic HSV of any preceding paragraph, wherein the HSV-2 immediate-early promoter of (f) is selected from the group consisting of ICP0, ICP4, and ICP27.
24. The oncolytic HSV of any preceding paragraph, wherein the HSV-2 immediate-early promoter of (f) is tet operator-containing.
25. The oncolytic HSV of any preceding paragraph, wherein the HSV is tetracycline or doxycycline-regulatable.
26. An oncolytic Herpes Simplex Virus (HSV) comprising recombinant DNA, wherein the recombinant DNA does not encode functional ICP0 or ICP34.5 genes; and encodes a functional mmTGF-β2-7 M fragment sequence.
27. An oncolytic Herpes Simplex Virus (HSV) comprising recombinant DNA, wherein the recombinant DNA does not encode functional ICP0; and encodes a functional mmTGF-β2-7 M fragment sequence.
28. The oncolytic HSV of any preceding paragraph, wherein the HSV further encodes fusogenic activity.
29. The oncolytic HSV of any preceding paragraph, wherein the HSV is tetracycline or doxycycline-regulatable.
30. An oncolytic virus encoding a functional mmTGF-β2-7 M fragment sequence.
31. A recombinant virus encoding a functional mmTGF-β2-7 M fragment sequence.
32. A composition comprising a virus of any of any preceding paragraph.
33. The composition of any preceding paragraph, further comprising a pharmaceutically acceptable carrier.
34. A cell expressing any of the viruses of any preceding paragraph or composition of any preceding paragraph.
35. The cell of any preceding paragraph, wherein the cell is mammalian.
36. The cell of any preceding paragraph, wherein the cell is a cancer cell or an immune cell.
37. The cell of any preceding paragraph, wherein the immune cell is a B cell or T cell.
38. The cell of any preceding paragraph, wherein the cell expresses high levels of mmTGF-β2-7 M.
39. A method for treating cancer, the method comprising administering the virus of any preceding paragraph or the composition of any preceding paragraph to a subject having cancer.
40. The method of any preceding paragraph, wherein the cancer is a solid tumor.
41. The method of any preceding paragraph, wherein the tumor is benign or malignant.
42. The method of any preceding paragraph, wherein the subject is diagnosed or has been diagnosed as having cancer is selected from the list consisting of a carcinoma, a melanoma, a sarcoma, a germ cell tumor, and a blastoma.
43. The method of any preceding paragraph, wherein the subject is diagnosed or has been diagnosed as having a cancer selected from the group consisting of non-small-cell lung cancer, bladder cancer, breast cancer, brain cancer, colon cancer, prostate cancer, liver cancer, lung cancer, ovarian cancer, skin cancer, head and neck cancer, kidney cancer, and pancreatic cancer.
44. The method of any preceding paragraph, wherein the cancer is metastatic.
45. The method of any preceding paragraph, further comprising administering an agent that regulates the tet operator-containing promoter.
46. The method of any preceding paragraph, wherein the agent is doxycycline or tetracycline.
47. The method of any preceding paragraph, wherein the agent is administered locally or systemically.
48. The method of any preceding paragraph, wherein the systemic administration is oral administration.
49. The method of any preceding paragraph, wherein the virus or composition is administered directly to the tumor.
50. A hybrid nucleic acid sequence containing a sequence of a therapeutic antibody and mmTGF-β2-7 M, wherein mmTGF-β2-7 M is fused to a Fc domain of the therapeutic antibody.
51. The hybrid nucleic acid sequence of any preceding paragraph, wherein the therapeutic antibody sequence is a sequence of an immunotherapeutic antibody.
52. The hybrid nucleic acid sequence of any preceding paragraph, wherein the therapeutic antibody sequence is a sequence selected from the list consisting of an anti-PD-1 antibody, an anti-PD-L1 antibody, an anti-Tim3 antibody, an anti-anti-CTLA4 antibody, and anti-TDM-1 antibody, and an anti-TIGIT antibody.
53. A polypeptide encoded by the hybrid nucleic acid of any preceding paragraph.
54. A vector expressing any of the hybrid nucleic acid of any preceding paragraph or polypeptides of any preceding paragraph.
55. A chimeric antigen receptor (CAR) polypeptide comprising at least one of
a. an extracellular domain comprising a dominant-negative TGF-β mutant sequence;
b. a transmembrane domain;
c. a co-stimulatory domain; and
d. an intracellular signaling domain.
56. The CAR polypeptide of any preceding paragraph, wherein the dominant-negative TGF-β mutant sequence is a mmTGF-β2-7 M fragment sequence.
57. A nucleic acid encoding the CAR polypeptide of any preceding paragraph.
58. A mammalian cell comprising:
a. the CAR polypeptide of any preceding paragraph; or
b. a nucleic acid encoding of any preceding paragraph.
59. The cell of any preceding paragraph, wherein the cell is a T cell.
60. The cell of any preceding paragraph, wherein the cell is a human cell.
61. The cell of any preceding paragraph, further comprising at least a second CAR polypeptide.
62. The cell of any preceding paragraph, where the at least second CAR polypeptide comprises an extracellular domain comprising a sequence that binds a checkpoint inhibitor.
63. The cell of any preceding paragraph, where the checkpoint inhibitor is selected from the group selected from: PD-L1, PD-1, TIGIT, TIM3, and CTLA4.
64. The cell of any preceding paragraph, wherein the cell is obtained from an individual having or diagnosed as having cancer.
65. A method of treating cancer in a subject in need thereof, the method comprising administering the cell of any preceding paragraph to the subject.
66. A method of treating cancer in a subject in need thereof, the method comprising:
a. engineering a T cell to comprise the CAR polypeptide of any preceding paragraph or nucleic acid encoding of any preceding paragraph on the T cell surface; and
b. administering the engineered T cell to the subject.
67. The method of any preceding paragraph, wherein the engineered T cell further comprises at least a second CAR polypeptide.
68. The method of any preceding paragraph, further comprising administering at least one additional anti-cancer therapeutic.
69. An oncolytic Herpes Simplex Virus (HSV) comprising recombinant DNA, wherein the recombinant DNA does not encode functional ICP0 and ICP34.5 genes; and encodes a functional mmTGF-β2-7 M fragment sequence.

### Example 1

### Introduction

Immune checkpoint blockade (ICB) represents an exciting new paradigm for the treatment of various cancers. The response rates to ICB, however, is generally between 10-35% (Bellmunt et al., 2017; Powles et al., 2018; Zou et al., 2016), leaving a larger proportion of patients unresponsiveness to ICB therapy. Immune suppressive tumor microenvironment presents one of the major obstacles that markedly limits the effectiveness of ICB in cancer immunotherapy. TGF-β plays a key role in promoting and maintenance of immune suppressive state of the tumor microenvironment (Bollard et al., 2002; Gorelik and Flavell, 2002; Loffek, 2018; Massague, 2008; Wrzesinski et al., 2007; Zhang et al., 2016). Over expression of TGF-β has been detected in a variety of human cancer types and is associated with poor prognosis (Calon et al., 2015; Dong and Blobe, 2006; Haque and Morris, 2017; Lin and Zhao, 2015; Mariathasan et al., 2018; Massague, 2008; Wikstrom et al., 1998; Wrzesinski et al., 2007).

Studies have revealed that TGF-β suppresses the Th1 response and CD8+ T cell activity, while promoting CD4⁺CD25⁺ T-reg cell function (Chen et al., 2005; Fantini et al., 2004; Loffek, 2018; Mariathasan et al., 2018; Tauriello et al., 2018; Verrecchia and Redini, 2018). Additionally, TGF-β inhibits dendritic cell maturation and antigen presentation, and anti-tumoral activity of NK cells, M1 macrophages and N1 neutrophils (Fridlender et al., 2009; Gong et al., 2012; Kmeta et al., 2017; Loffek, 2018; Luo et al., 2006; Verrecchia and Redini, 2018; Zhang et al., 2016; Zheng et al., 2017). Mariathasan et al. have recently shown that TGF-β attenuates tumor response to PD-L1 immune blockade by preventing T-cell infiltration, while blocking TGF-β signaling in the tumor microenvironment led to strong enhancement in anti-tumor T cell response and tumor regression (Mariathasan et al., 2018). Thus, inhibition of TGF-β signaling in the tumor microenvironment has gained a significant interest in cancer immunotherapy (Bendle et al., 2013; Biswas et al., 2007; de Gramont et al., 2017; Haque and Morris, 2017; Hutzen et al., 2017; Knudson et al., 2018; Loffek, 2018; Muraoka et al., 2002; Strauss et al., 2018) (Ahn Myung-ju, Barlesi F et al., 2019-J Clinical Oncology-Abstract; Strauss J et al. and Gulley J, 2019-Cancer Res).

Over the years, various molecules that are capable of blocking TGF-β signaling have been developed, including small molecules, peptides, and soluble forms of TGF-β type II receptor (TβRII), and anti-TGF-β1 antibodies (Biswas et al., 2007; Bottinger et al., 1997; de Gramont et al., 2017; Gil-Guerrero et al., 2008; Haque and Morris, 2017; Muraoka et al., 2002; Qin et al., 2016; Rowland-Goldsmith et al., 2001; Tian et al., 2015; Tojo et al., 2005). Recently, Kim et al. developed a novel completely non-functional monomeric form of dominant-negative TGF-β polypeptide, mmTGF-β2-7 M, that exhibits high affinity to the TGF-β type II receptor (TβRII), while is incapable of binding to TGF-β type I receptor (TβRI) (Kim et al., 2017). Moreover, mmTGF-β2-7 M produced and purified from E. Coli is highly effective in blocking TGF-β1, TGF-β2, and TGF-β3 signaling in TGF-β reporter cell line (Kim et al., 2017). To date, no report has described expression of mmTGF-β2-7 M in mammalian cells. It, thus, remains to be determined whether the mmTGF-β2-7 M expressed from mammalian cells can function as a potent dominant-negative mutant capable of blocking the signaling of TGF-β.

QREO5-F is the second generation of fusogenic tetracycline-regulatable oncolytic HSV-1 recombinant virus recently developed by the inventors. Infection of multiple human cancer cell types with QREO5-F lead to 35,000- to 5×10⁷-fold tetracycline-dependent progeny virus production, while little viral replication and virus-associated cytotoxicity are observed in infected growing as well as growth-arrested normal human fibroblasts. QREO5-F is highly effective against pre-established Hep1-6 hepatoma and CT26.WT colon carcinoma tumor in immune-competent mice. Importantly, QREO5-F virotherapy can lead to induction of an effective tumor-specific immunity that can prevent the tumor growth following re-challenge tumor-free mice with the same type of tumor cells. In light of the key roles of TGF-β signaling in tumor biology and its potent immune suppressive activities, it was specifically contemplated that the therapeutic efficacy of QREO5-F in cancer immunotherapy could be further enhanced when armed with de novo expression of mmTGF-β2-7 M in the localized tumor microenvironment.

Construction and characterization of QREOF-lacZ, a QREO5-F derived recombinant that encodes the lacZ gene under the control of the HSV-2 ICP0 immediate-early promoter at the intergenic region of the HSV-1 UL26 and UL27 genes.
Description of Plasmids pQUL2627-TO and pQUL2627-lacZ

pQUL2627-TO contains a synthesized DNA fragment consisting of 1) HSV-1 DNA sequence consisting of 963 bp upstream of HSV-1 UL26 poly A signal to 30 bp downstream of UL26 poly A signal sequence, 2) DNA sequence containing a modified HSV-2 ICP0 promoter in which the HSV-2 TATA element is changed to the HCMV TATATAA followed by two tandem tet operators as described by Yao et al. (Yao et al., 1998), MCS, and the SV40 poly A signal sequence, and 3) HSV-1 DNA sequence consisting of 59 bp downstream of the HSV-1 UL27 poly A signal to 935 bp upstream of UL27 poly A signal. pQUL2627-v is a pQUL2627-TO-derived plasmid without the tet operator sequence. pQUL2627-lacZ is a pQUL2627-v-derived plasmid that encodes the lacZ gene under the control of the modified HSV-2 ICP0 promoter.

### Construction and Characterization of QREOF-lacZ

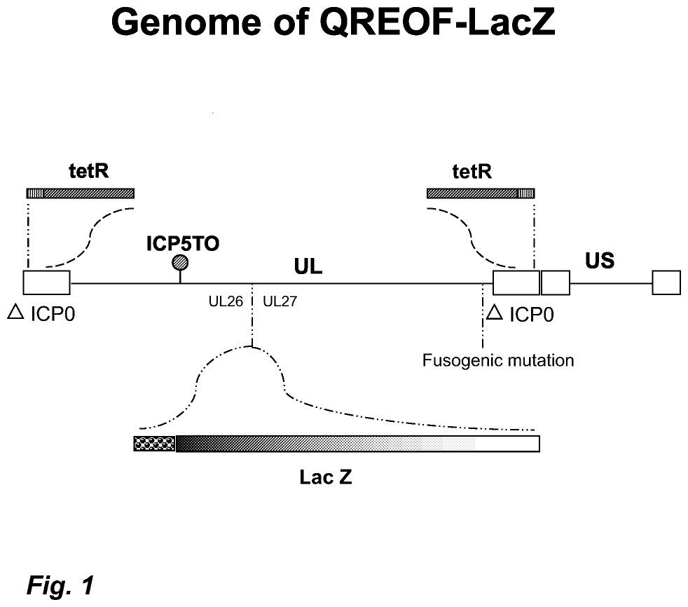

QREOF-lacZ is a QREO5-F-derived recombinant virus, in which the lacZ gene under the control of the modified HSV-2 ICP0 promoter is inserted into the intergenic region of UL26 and UL27 genes (FIG. 1). QREOF-lacZ was generated by co-transfecting U2OS cells with Sap I/Xmn I-linearized pQUL2627-lacZ and infectious QREO5-F viral DNA through Lipofectamine 2000-mediated transfection (Akhrameyeva et al., 2011). The lacZ-expressing viruses were then selected and plaque-purified on U2OS cells in the presence of 5-bromo-4-chloro-3-indolyl-b-D-galactopyranoside (X-Gal).

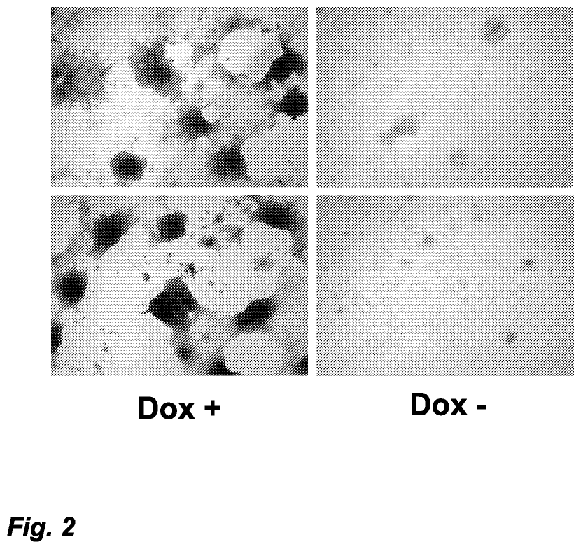

QREOF-lacZ is a third-round plaque-purified QREO5-F-derived recombinant virus that exhibits uniform blue fusogenic plaques in U2OS cells and the ICP0-expressing Vero cell line, Q0-19 cells. The result presented in FIG. 2 shows that both the expression of the lacZ gene and replication of QREOF-lacZ can be tightly regulated in QREOF-lacZ infected cells.

Construction and characterization of QREO-DNT, a QREOF-lacZ derived recombinant that encodes the dominant-negative TGF-β mutant, mmTGF-β2-7 M, under the control of the modified HSV-2 ICP4 immediate-early promoter at the intergenic region of the HSV-1 UL26 and UL27 genes.
Description of Plasmid pQUL2627-TGFDN and In Vitro Expression of mmTGF-β2-7 M by Transient Transfection Assay

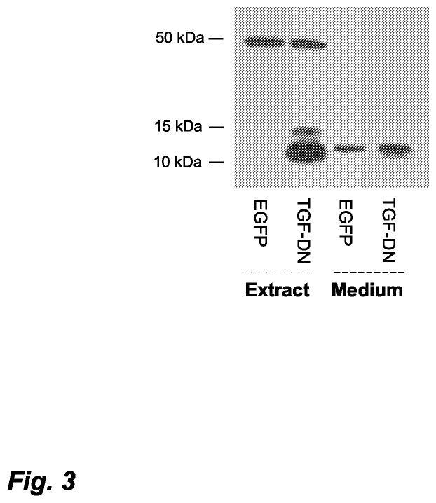

pQUL2627-TGF-DN was constructed by replacing the HSV-2 ICP0/lacZ gene-containing DNA fragment in pQUL2627-lacZ with a synthesized DNA fragment consisting of the codon optimized mmTGF-β2-7 M with the HSV-1 gD signal peptide under the control of the tetO-containing HSV-2 ICP4/TO promoter. mmTGF-β2-7 M consists of 92 amino acids. To assess the expression of mmTGF-β2-7 M, U2OS cells were mock-transfected or transfected with pQUL26.27-TGF-DN, or pICP6-eGFP, an eGFP-expressing plasmid that encodes eGFP under the control of the HSV-1 ICP6 promoter. The western blot analyses presented in FIG. 3 show that while a protein with a MW close to 50 kDa, representing the full-length TGF-β1 precursor (390 amino acids), is present in both pICP6-EGFP and pQUL26.27-TGFDN transfected cell extracts, only pQUL26.27-TGF-DN-transfected cells yields a protein with a MW between 11-12 kDa that was strongly recognized by the TGF β1-specific antibody. No mature form of TGF-β1 could be detected in eGFP-transfected cell extract. The mature TGF-β1, however, is detectable in both extracellular medium collected from pICP6-eGFP and pQUL26.27-TGF-DN transfected cells.

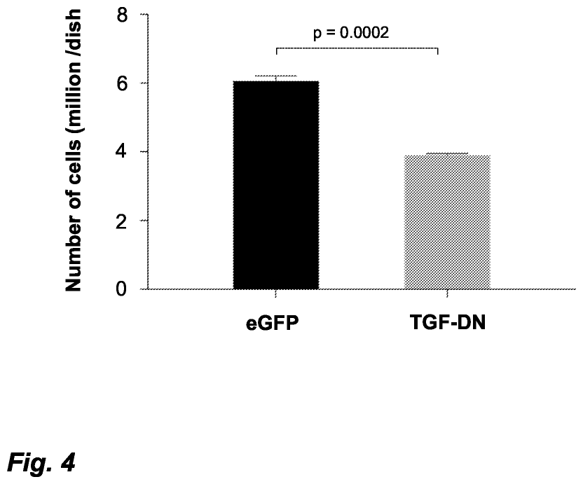

When examined the transfected cells described above under the phase contrast light and fluorescence microscope, it was observed that about 35-40% cells are eGFP positive in pICP6-eGFP transfected dishes at 40 h post-transfection. While eGFP transfected cells were morphologically similar to that of mock-transfected cells from 28 h to 70 h post-transfection, U2OS cells transfected with pQUL2627-TGF-DN appeared flat, markedly enlarged and significantly stressed at 48 h and 70 h post-transfection. Moreover, it appeared that dishes transfected with pQUL2627-TGF-DN contained significantly less cells than pICP6-EGFP-transfected dishes. These observations were further confirmed by an independent experiment presented in FIG. 4, which shows that the number of cells per dish in dishes transfected with pICP6-eGFP is close to 1.6-fold higher than that of dishes transfected with pQUL2627-TGF-DN, indicating that mmTGF-β2-7 M expressed from the transfected cells can effectively block the TGF-β signaling in U2OS cells, consistent with the previous studies that TGF-β signaling is required for the proliferation, migration and invasiveness of osteosarcoma cells, including U2OS cells (Li et al., 2014; Matsuyama et al., 2003; Verrecchia and Redini, 2018).

### Construction and Characterization of QREO-DNT

QREO-DNT is a QREOF-lacZ-derived recombinant virus, in which the lacZ gene under the control of the modified HSV-2 ICP0 promoter is replaced by the DNA fragment encoding the codon optimized codon optimized mmTGF-β2-7 M under the control of the HSV-2 ICP4/TO promoter sequence.

QREO-DNT was generated by co-transfecting U2OS cells with Nde I/Bbs I-linearized pQUL2627-TGF-DN and infectious QREOF-lacZ viral DNA by Lipofectamine 2000. The mmTGF-β2-7 M-expressing viruses were selected and plaque-purified on U2OS cells in the presence of X-Gal. In short, progeny viruses of the transfection were screened for the recombinational replacement of the LacZ gene of QREOF-lacZ with the HSV2 ICP4TO/mmTGF-β2-7 M-containing DNA sequence by standard plaque assays. Plaques were stained with X-Gal at 72 h post-infection. White plaques, reflecting the replacement of the LacZ gene by the mmTGF-β2-7 M DNA-encoding sequence, were isolated. The replacement of the lacZ gene with the mmTGF-f32-7 M-encoding DNA sequence at the UL26 and UL27 intergenic region was confirmed by PCR analysis with the primers specific for the HSV2 ICP4TO promoter sequence and the UL27 flanking sequence. QREO-DNT is a second-round plaque-purified mmTGF-β2-7 M-encoding recombinant virus that exhibits uniform white fusogenic plaques in U2OS cells and ICP0-expressing Vero cell line, Q0-19 cells.

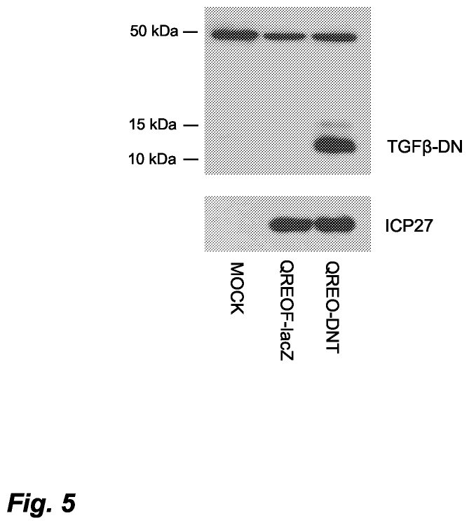

The ability of QREO-DNT to efficiently express dominant-negative form of TGF-β (TGF-DN) was assessed in U2OS cells at a MOI of 3 PFU/cell in the presence of doxycycline. The western blot analyses presented in FIG. 5 show that, while similar levels of ICP27 are detected in QREOF-lacZ- and QREO-DNT-infected cells, only QREO-DNT expresses a protein with a MW around 11-12 kDa that reacted strongly with the anti-TGF-β1-specific antibody. As expected, the full-length precursor form of TGF-β1 is detectable in both mock-infected and infected cell extracts. Notably, a very light protein band with a MW slightly higher than that of mmTGF-β2-7 M was detected in mock-infected cell extract, which likely represents the mature form of TGF-β1 (112 amino acids). Collectively, the results presented in FIG. 5 demonstrate that QREO-DNT is capable of expressing high-levels of dominant-negative form of TGF-β1, mmTGF-β2-7 M.

### REFERENCES

Akhrameyeva, N. V., Zhang, P., Sugiyama, N., Behar, S. M., and Yao, F. (2011). Development of a glycoprotein D-expressing dominant-negative and replication-defective herpes simplex virus 2 (HSV-2) recombinant viral vaccine against HSV-2 infection in mice. J Virol 85, 5036-5047.
Bellmunt, J., de Wit, R., Vaughn, D. J., Fradet, Y., Lee, J. L., Fong, L., Vogelzang, N. J., Climent, M. A., Petrylak, D. P., Choueiri, T. K., et al. (2017). Pembrolizumab as Second-Line Therapy for Advanced Urothelial Carcinoma. N Engl J Med 376, 1015-1026.
Bendle, G. M., Linnemann, C., Bies, L., Song, J. Y., and Schumacher, T. N. (2013). Blockade of TGF-beta signaling greatly enhances the efficacy of TCR gene therapy of cancer. J Immunol 191, 3232-3239.
Biswas, S., Guix, M., Rinehart, C., Dugger, T. C., Chytil, A., Moses, H. L., Freeman, M. L., and Arteaga, C. L. (2007). Inhibition of TGF-beta with neutralizing antibodies prevents radiation-induced acceleration of metastatic cancer progression. J Clin Invest 117, 1305-1313.
Bollard, C. M., Rossig, C., Calonge, M. J., Huls, M. H., Wagner, H. J., Massague, J., Brenner, M. K., Heslop, H. E., and Rooney, C. M. (2002). Adapting a transforming growth factor beta-related tumor protection strategy to enhance antitumor immunity. Blood 99, 3179-3187.
Bottinger, E. P., Jakubczak, J. L., Roberts, I. S., Mumy, M., Hemmati, P., Bagnall, K., Merlino, G., and Wakefield, L. M. (1997). Expression of a dominant-negative mutant TGF-beta type II receptor in transgenic mice reveals essential roles for TGF-beta in regulation of growth and differentiation in the exocrine pancreas. EMBO J 16, 2621-2633.
Calon, A., Lonardo, E., Berenguer-Llergo, A., Espinet, E., Hernando-Momblona, X., Iglesias, M., Sevillano, M., Palomo-Ponce, S., Tauriello, D. V., Byrom, D., et al. (2015). Stromal gene expression defines poor-prognosis subtypes in colorectal cancer. Nat Genet 47, 320-329.
Chen, M. L., Pittet, M. J., Gorelik, L., Flavell, R. A., Weissleder, R., von Boehmer, H., and Khazaie, K. (2005). Regulatory T cells suppress tumor-specific CD8 T cell cytotoxicity through TGF-beta signals in vivo. Proc Natl Acad Sci USA 102, 419-424.
de Gramont, A., Faivre, S., and Raymond, E. (2017). Novel TGF-beta inhibitors ready for prime time in onco-immunology. Oncoimmunology 6, e1257453.
Dong, M., and Blobe, G. C. (2006). Role of transforming growth factor-beta in hematologic malignancies. Blood 107, 4589-4596.
Fantini, M. C., Becker, C., Monteleone, G., Pallone, F., Galle, P. R., and Neurath, M. F. (2004). Cutting edge: TGF-beta induces a regulatory phenotype in CD4+CD25− T cells through Foxp3 induction and down-regulation of Smad7. J Immunol 172, 5149-5153.
Fridlender, Z. G., Sun, J., Kim, S., Kapoor, V., Cheng, G., Ling, L., Worthen, G. S., and Albelda, S. M. (2009). Polarization of tumor-associated neutrophil phenotype by TGF-beta: “N1” versus “N2” TAN. Cancer Cell 16, 183-194.
Gil-Guerrero, L., Dotor, J., Huibregtse, I. L., Casares, N., Lopez-Vazquez, A. B., Rudilla, F., Riezu-Boj, J. I., Lopez-Sagaseta, J., Hermida, J., Van Deventer, S., et al. (2008). In vitro and in vivo down-regulation of regulatory T cell activity with a peptide inhibitor of TGF-beta1. J Immunol 181, 126-135.
Gong, D., Shi, W., Yi, S. J., Chen, H., Groffen, J., and Heisterkamp, N. (2012). TGFbeta signaling plays a critical role in promoting alternative macrophage activation. BMC Immunol 13, 31.
Gorelik, L., and Flavell, R. A. (2002). Transforming growth factor-beta in T-cell biology. Nat Rev Immunol 2, 46-53.
Haque, S., and Morris, J. C. (2017). Transforming growth factor-beta: A therapeutic target for cancer. Hum Vaccin Immunother 13, 1741-1750.
Hutzen, B., Chen, C. Y., Wang, P. Y., Sprague, L., Swain, H. M., Love, J., Conner, J., Boon, L., and Cripe, T. P. (2017). TGF-beta Inhibition Improves Oncolytic Herpes Viroimmunotherapy in Murine Models of Rhabdomyosarcoma. Mol Ther Oncolytics 7, 17-26.
Kim, S. K., Barron, L., Hinck, C. S., Petrunak, E. M., Cano, K. E., Thangirala, A., Iskra, B., Brothers, M., Vonberg, M., Leal, B., et al. (2017). An engineered transforming growth factor beta (TGF-beta) monomer that functions as a dominant negative to block TGF-beta signaling. J Biol Chem 292, 7173-7188.
Knudson, K. M., Hicks, K. C., Luo, X., Chen, J. Q., Schlom, J., and Gameiro, S. R. (2018). M7824, a novel bifunctional anti-PD-L1/TGFbeta Trap fusion protein, promotes anti-tumor efficacy as monotherapy and in combination with vaccine. Oncoimmunology 7, e1426519.
Kmeta, T., Gillgrass, A., Poznanski, S., Chew, M., Lee, A. J., Kolb, M., and Ashkar, A. A. (2017). M2-polarized and tumor-associated macrophages alter N K cell phenotype and function in a contact-dependent manner. J Leukoc Biol 101, 285-295.
Li, F., Li, S., and Cheng, T. (2014). TGF-beta1 promotes osteosarcoma cell migration and invasion through the miR-143-versican pathway. Cell Physiol Biochem 34, 2169-2179.
Lin, R. L., and Zhao, L. J. (2015). Mechanistic basis and clinical relevance of the role of transforming growth factor-beta in cancer. Cancer Biol Med 12, 385-393.
Loffek, S. (2018). Transforming of the Tumor Microenvironment: Implications for TGF-beta Inhibition in the Context of Immune-Checkpoint Therapy. J Oncol 2018, 9732939.
Luo, Y., Zhou, H., Krueger, J., Kaplan, C., Lee, S. H., Dolman, C., Markowitz, D., Wu, W., Liu, C., Reisfeld, R. A., et al. (2006). Targeting tumor-associated macrophages as a novel strategy against breast cancer. J Clin Invest 116, 2132-2141.
Mariathasan, S., Turley, S. J., Nickles, D., Castiglioni, A., Yuen, K., Wang, Y., Kadel, E. E., III, Koeppen, H., Astarita, J. L., Cubas, R., et al. (2018). TGFbeta attenuates tumour response to PD-L1 blockade by contributing to exclusion of T cells. Nature 554, 544-548.
Massague, J. (2008). TGFbeta in Cancer. Cell 134, 215-230.
Matsuyama, S., Iwadate, M., Kondo, M., Saitoh, M., Hanyu, A., Shimizu, K., Aburatani, H., Mishima, H. K., Imamura, T., Miyazono, K., et al. (2003). SB-431542 and Gleevec inhibit transforming growth factor-beta-induced proliferation of human osteosarcoma cells. Cancer Res 63, 7791-7798.
Muraoka, R. S., Dumont, N., Ritter, C. A., Dugger, T. C., Brantley, D. M., Chen, J., Easterly, E., Roebuck, L. R., Ryan, S., Gotwals, P. J., et al. (2002). Blockade of TGF-beta inhibits mammary tumor cell viability, migration, and metastases. J Clin Invest 109, 1551-1559.
Powles, T., Duran, I., van der Heijden, M. S., Loriot, Y., Vogelzang, N. J., De Giorgi, U., Oudard, S., Retz, M. M., Castellano, D., Bamias, A., et al. (2018). Atezolizumab versus chemotherapy in patients with platinum-treated locally advanced or metastatic urothelial carcinoma (IMvigor211): a multicentre, open-label, phase 3 randomised controlled trial. Lancet 391, 748-757.
Qin, T., Barron, L., Xia, L., Huang, H., Villarreal, M. M., Zwaagstra, J., Collins, C., Yang, J., Zwieb, C., Kodali, R., et al. (2016). A novel highly potent trivalent TGF-beta receptor trap inhibits early-stage tumorigenesis and tumor cell invasion in murine Pten-deficient prostate glands. Oncotarget 7, 86087-86102.
Rowland-Goldsmith, M. A., Maruyama, H., Kusama, T., Ralli, S., and Korc, M. (2001). Soluble type II transforming growth factor-beta (TGF-beta) receptor inhibits TGF-beta signaling in COLO-357 pancreatic cancer cells in vitro and attenuates tumor formation. Clin Cancer Res 7, 2931-2940.
Strauss, J., Heery, C. R., Schlom, J., Madan, R. A., Cao, L., Kang, Z., Lamping, E., Marte, J. L., Donahue, R. N., Grenga, I., et al. (2018). Phase I Trial of M7824 (MSB0011359C), a Bifunctional Fusion Protein Targeting PD-L1 and TGFbeta, in Advanced Solid Tumors. Clin Cancer Res 24, 1287-1295.
Tauriello, D. V. F., Palomo-Ponce, S., Stork, D., Berenguer-Llergo, A., Badia-Ramentol, J., Iglesias, M., Sevillano, M., Ibiza, S., Canellas, A., Hemando-Momblona, X., et al. (2018). TGFbeta drives immune evasion in genetically reconstituted colon cancer metastasis. Nature 554, 538-543.
Tian, H., Liu, J., Chen, J., Gatza, M. L., and Blobe, G. C. (2015). Fibulin-3 is a novel TGF-beta pathway inhibitor in the breast cancer microenvironment. Oncogene 34, 5635-5647.
Tojo, M., Hamashima, Y., Hanyu, A., Kajimoto, T., Saitoh, M., Miyazono, K., Node, M., and Imamura, T. (2005). The ALK-5 inhibitor A-83-01 inhibits Smad signaling and epithelial-to-mesenchymal transition by transforming growth factor-beta. Cancer Sci 96, 791-800.
Verrecchia, F., and Redini, F. (2018). Transforming Growth Factor-beta Signaling Plays a Pivotal Role in the Interplay Between Osteosarcoma Cells and Their Microenvironment. Front Oncol 8, 133.
Wikstrom, P., Stattin, P., Franck-Lissbrant, I., Damber, J. E., and Bergh, A. (1998). Transforming growth factor beta1 is associated with angiogenesis, metastasis, and poor clinical outcome in prostate cancer. Prostate 37, 19-29.
Wrzesinski, S. H., Wan, Y. Y., and Flavell, R. A. (2007). Transforming growth factor-beta and the immune response: implications for anticancer therapy. Clin Cancer Res 13, 5262-5270.
Yao, F., Svensjo, T., Winkler, T., Lu, M., Eriksson, C., and Eriksson, E. (1998). Tetracycline repressor, tetR, rather than the tetR-mammalian cell transcription factor fusion derivatives, regulates inducible gene expression in mammalian cells. Hum Gene Ther 9, 1939-1950.
Zhang, F., Wang, H., Wang, X., Jiang, G., Liu, H., Zhang, G., Wang, H., Fang, R., Bu, X., Cai, S., et al. (2016). TGF-beta induces M2-like macrophage polarization via SNAIL-mediated suppression of a pro-inflammatory phenotype. Oncotarget 7, 52294-52306.
Zheng, X., Turkowski, K., Mora, J., Brune, B., Seeger, W., Weigert, A., and Savai, R. (2017). Redirecting tumor-associated macrophages to become tumoricidal effectors as a novel strategy for cancer therapy. Oncotarget 8, 48436-48452.
Zou, W., Wolchok, J. D., and Chen, L. (2016). PD-L1 (B7-H1) and PD-1 pathway blockade for cancer therapy: Mechanisms, response biomarkers, and combinations. Sci Transl Med 8, 328rv324.

### Example 2

### DNA Sequences and Amino Acid Sequences

Transcriptional cassette for targeting gene of interest under the control of the modified tetO-
bearing HSV-2 ICPO promoter into the HSV-1 UL26/UL27 locus:
tcccattaca accagctcgt cgccggccac gccgcgcccc aaccccagcc
gcattccgcg tttggtttcc cggctgcggc gggggccgtg gcctatgggc ctcacggcgc
gggtctttcc cagcattacc ctccccacgt cgcccatcag tatcccgggg tgctgttctc
gggacccagc ccactcgagg cgcagatagc cgcgttggtg ggggccatag ccgcggaccg
ccaggcgggc ggtcagccgg ccgcgggaga ccctggggtc cgggggtcgg gaaagcgtcg
ccggtacgag gcggggccgt cggagtccta ctgcgaccag gacgaaccgg acgcggacta
cccgtactac cccggggagg ctcgaggcgg gccgcgcggg gtcgactctc ggcgcgcggc
ccgccagtct cccgggacca acgagaccat cacggcgctg atgggggcgg tgacgtctct
gcagcaggaa ctggcgcaca tgcgggctcg gaccagcgcc ccctatggaa tgtacacgcc
ggtggcgcac tatcgccctc aggtggggga gccggaacca acaacgaccc acccggccct
ttgtcccccg gaggccgtgt atcgcccccc accacacagc gccccctacg gtcctcccca
gggtccggcg tcccatgccc ccactccccc gtatgcccca gctgcctgcc cgccaggccc
gccaccgccc ccatgtcctt ccacccagac gcgcgcccct ctaccgacgg agcccgcgtt
cccccccgcc gccaccggat cccaaccgga ggcatccaac gcggaggccg gggcccttgt
caacgccagc agcgcagcac acgtggacgt tgacacggcc cgcgccgccg atttgttcgt
ctctcagatg atgggggccc gctgattcgc cccggtcttt ggtaccatgg gatgtcttac
tgtatatctt tttaaataaa ccaggtaata ccaaataaga cccattggtg
TATGTTCTTTTTTTATTGGCGCGCCATATG cattcccgct tatgctaatg agatgcgttc
ggacggcccc tcgctccccg cgtaattact ccctcggggt tccgggttat gctgattact
ttcttggcag aacacgcaga gcctcgcgcg ccgccgggtg ggtgggctga tcggccccta
ttggtcccct gggcttccta gtatgctaat gatattctcc ccgggggcgg gcaccactca
gggccgcgcc ggcggggcgc cggggggact cccatctgcg tcggcggggg gcggcgcatg
ctaatgatat tcttggagta cacccggttg gtccccgggg acggggccgc cccgagaggg
ggggattccc tccctccgcc cccgccgggg cgcgcggcta ttgggggaat cgtaaatgcc
gcccctttgg gggagtggat aggcgccggg tatataagca gagctctccc tatcagtgat
agagatctcc ctatcagtga tagagatcgt cgacgagctc gcgAAGCTTA CCGGTCGATA
TCTATCGCGA GCTCGCCAAT TGCGAGATCT AGAAGCGGAA TTCTgcccgt CAGAACTTGT
TTATTGCAGC TTATAATGGT TACAAATAAA GCAATAGCAT CACAAATTTC ACAAATAAAG
CATTTTTTTC ACTGCATTCT AGTTGTGGTT TGTCCAAACT CATCAATGTA TCTTATCATG
TCTGGTTACC caaaaggcgt accaaacaaa aaaaccaaaa gatgcgcatg cggtttaaca
cccgtggttt ttatttacaa caaacccccc gtcacaggtc gtcctcgtcg gcgtcaccgt
ctttgttggg aacttgggtg tagttggtgt tgcggcgctt gcgcatgacc atgtcggtga
ccttggcgct gagcagcgcg ctcgtgccct tcttcttggc cttgtgttcc gtgcgctcca
tggccgacac cagggccatg taccgtatca tctccctggc ctcggctagc ttggcctcgt
caaagtcgcc gccctcctcg ccctccccgg acgcgtccgg gttggtgggg ttcttgagct
ccttggtggt tagagggtac agggccttca tggggttgct ctgcagccgc atgacgtaac
gaaaggcgaa gaaggccgcc gccaggccgg ccaggaccaa cagacccacg gccagcgccc
caaaggggtt ggacatgaag gaggacacgc ccgacacggc cgataccacg ccgcccacga
tgcccatcac caccttgccg accgcgcgcc ccaggtcgcc catcccctcg aagaacgcgc
ccaggcccgc gaacatggcg gcgttggcgt cggcgtggat gaccgtgtcg atgtcggcga
agcgcaggtc gtgcagctgg ttgcggcgct ggacctccgt gtagtccagc aggccgctgt
ccttgatctc gtggcgggtg tacacctcca gggggacaaa ctcgtgatcc tccagcatgg
tgatgttgag gtcgatgaag gtgctgacgg tggtgatgtc ggcgcggctc agctggtggg
agtacgcgta ctcctcgaag tacacgtagc ccccaccgaa ggtgaagtag cgccggtgtc
ccacggtgca cggctcgatc gcatcgcgcg tcagccgcag ctcgttgttc tcccccagct
gcccctcgac caacgggccc tggtcttcgt accgaaagct gaccaggggg (SEQ ID NO:
12)
DNA sequence consisting of 963 bp upstream of the poly A signal of UL26 to 30 bp down stream
of the UL26 poly A signal:
tcccattaca accagctcgt cgccggccac gccgcgcccc aaccccagcc
gcattccgcg tttggtttcc cggctgcggc gggggccgtg gcctatgggc ctcacggcgc
gggtctttcc cagcattacc ctccccacgt cgcccatcag tatcccgggg tgctgttctc
gggacccagc ccactcgagg cgcagatagc cgcgttggtg ggggccatag ccgcggaccg
ccaggcgggc ggtcagccgg ccgcgggaga ccctggggtc cgggggtcgg gaaagcgtcg
ccggtacgag gcggggccgt cggagtccta ctgcgaccag gacgaaccgg acgcggacta
cccgtactac cccggggagg ctcgaggcgg gccgcgcggg gtcgactctc ggcgcgcggc
ccgccagtct cccgggacca acgagaccat cacggcgctg atgggggcgg tgacgtctct
gcagcaggaa ctggcgcaca tgcgggctcg gaccagcgcc ccctatggaa tgtacacgcc
ggtggcgcac tatcgccctc aggtggggga gccggaacca acaacgaccc acccggccct
ttgtcccccg gaggccgtgt atcgcccccc accacacagc gccccctacg gtcctcccca
gggtccggcg tcccatgccc ccactccccc gtatgcccca gctgcctgcc cgccaggccc
gccaccgccc ccatgtcctt ccacccagac gcgcgcccct ctaccgacgg agcccgcgtt
cccccccgcc gccaccggat cccaaccgga ggcatccaac gcggaggccg gggcccttgt
caacgccagc agcgcagcac acgtggacgt tgacacggcc cgcgccgccg atttgttcgt
ctctcagatg atgggggccc gctgattcgc cccggtcttt ggtaccatgg gatgtcttac
tgtatatctt tttaaataaa ccaggtaata ccaaataaga cccattggtg (SEQ ID NO:
13)
DNA sequence consisting of 59 bp downstream of the UL27 poly A signal to 935 bp upstream of
the poly A signal of UL27:
caaaaggcgt accaaacaaa aaaaccaaaa gatgcgcatg cggtttaaca
cccgtggttt ttatttacaa caaacccccc gtcacaggtc gtcctcgtcg gcgtcaccgt
ctttgttggg aacttgggtg tagttggtgt tgcggcgctt gcgcatgacc atgtcggtga
ccttggcgct gagcagcgcg ctcgtgccct tcttcttggc cttgtgttcc gtgcgctcca
tggccgacac cagggccatg taccgtatca tctccctggc ctcggctagc ttggcctcgt
caaagtcgcc gccctcctcg ccctccccgg acgcgtccgg gttggtgggg ttcttgagct
ccttggtggt tagagggtac agggccttca tggggttgct ctgcagccgc atgacgtaac
gaaaggcgaa gaaggccgcc gccaggccgg ccaggaccaa cagacccacg gccagcgccc
caaaggggtt ggacatgaag gaggacacgc ccgacacggc cgataccacg ccgcccacga
tgcccatcac caccttgccg accgcgcgcc ccaggtcgcc catcccctcg aagaacgcgc
ccaggcccgc gaacatggcg gcgttggcgt cggcgtggat gaccgtgtcg atgtcggcga
agcgcaggtc gtgcagctgg ttgcggcgct ggacctccgt gtagtccagc aggccgctgt
ccttgatctc gtggcgggtg tacacctcca gggggacaaa ctcgtgatcc tccagcatgg
tgatgttgag gtcgatgaag gtgctgacgg tggtgatgtc ggcgcggctc agctggtggg
agtacgcgta ctcctcgaag tacacgtagc ccccaccgaa ggtgaagtag cgccggtgtc
ccacggtgca cggctcgatc gcatcgcgcg tcagccgcag ctcgttgttc tcccccagct
gcccctcgac caacgggccc tggtcttcgt accgaaagct gaccaggggg (SEQ ID NO:
14)
TetO-bearing modified HSV-2 ICPO promoter plus designed mcs followed by sv40 poly A signal
sequence:
cattcccgct tatgctaatg agatgcgttc ggacggcccc tcgctccccg
cgtaattact ccctcggggt tccgggttat gctgattact ttcttggcag aacacgcaga
gcctcgcgcg ccgccgggtg ggtgggctga tcggccccta ttggtcccct gggcttccta
gtatgctaat gatattctcc ccgggggcgg gcaccactca gggccgcgcc ggcggggcgc
cggggggact cccatctgcg tcggcggggg gcggcgcatg ctaatgatat tcttggagta
cacccggttg gtccccgggg acggggccgc cccgagaggg ggggattccc tccctccgcc
cccgccgggg cgcgcggcta ttgggggaat cgtaaatgcc gcccctttgg gggagtggat
aggcgccggg tatataagca gagctctccc tatcagtgat agagatctcc ctatcagtga
tagagatcgt cgacgagctc gcgAAGCTTA CCGGTCGATA TCTATCGCGA GCTCGCCAAT
TGCGAGATCT AGAAGCGGAA TTCTgcccgt
CAGAACTTGTTTATTGCAGCTTATAATGGTTACAAATAAAGCAATAGCATCACAAATTTCACAAATAA
AGCATTTTTTTCACTGCATTCTAGTTGTGGTTTGTCCAAACTCATCAATGTATCTTATCATGTCTG
(SEQ ID NO: 15)
SV40 Poly A signal sequence:
CAGAACTTGTTTATTGCAGCTTATAATGGTTACAAATAAAGCAATAGCATCACAAAT
TTCACAAATAAAGCATTTTTTTCACTGCATTCTAGTTGTGGTTTGTCCAAACTCATCAATGTATCTTA
TCATGTCTG (SEQ ID NO: 16)
Codon Optimized mmTGF-P2-7M with HSV-1 gD signal peptide under the control of the HSV-
2 ICP4/TO promoter sequence:
ggcgcgccgggccggcgggggccaacgggagcgcggggccggcatctcattaccacg
aacccggaagggcaggggagcgagcccgcccgcgacgagggtctcattagcatcgcgggcggaagcgg
aagccgcccgcgccgggcgctaatgagatgccgcgcgggcggagcggcggcggcgcgaccaacgggcc
gccgccacggacgcggacgcgcgggcgtcggggcggggccgcgcataatgcggttccacctgggggcg
gaaccccggcgagccggggcgcggcggcgtcgatcgctcctcctccgcgtcctcctcctttccccccg
ccccgcgcgccccgaggacTATATGAGCCGAGCTCTCCCTATCAGTGATAGAGATCTCCCTATCAGTG
ATAGAGATCGAGCTCGCGTGTGCATCGCGTATCACCCAAGCTTgccaccATGGGCGGCGCCGCCGCCC
GCCTGGGCGCCGTGATCCTGTTCGTGGTGATCGTGGGCCTGCACGGCGTGCGCGGCGCCCTGGACGCC
GCCTACTGCTTCCGCAACGTGCAGGACAACTGCTGCCTGCGCCCCCTGTACATCGACTTCCGCAAGGA
CCTGGGCTGGAAGTGGATCCACGAGCCCAAGGGCTACAACGCCAACTTCTGCGCCGGCGCCTGCCCCT
ACCGCGCCAGCAAGAGCCCCAGCTGCGTGAGCCAGGACCTGGAGCCCCTGACCATCGTGTACTACGTG
GGCCGCAAGCCCAAGGTGGAGCAGCTGAGCAACATGATCGTGAAGAGCTGCAAGTGCAGCTAAgaatt
c (SEQ ID NO: 17)
HSV-2 ICP4/TO promoter sequence:
ggcgcgccgggccggcgggggccaacgggagcgcggggccggcatctcattaccacg
aacccggaagggcaggggagcgagcccgcccgcgacgagggtctcattagcatcgcgggcggaagcgg
aagccgcccgcgccgggcgctaatgagatgccgcgcgggcggagcggcggcggcgcgaccaacgggcc
gccgccacggacgcggacgcgcgggcgtcggggcggggccgcgcataatgcggttccacctgggggcg
gaaccccggcgagccggggcgcggcggcgtcgatcgctcctcctccgcgtcctcctcctttccccccg
ccccgcgcgccccgaggacTATATGAGCCGAGCTCTCCCTATCAGTGATAGAGATCTCCCTATCAGTG
ATAGAGATCGAGCTCGCGTGTGCATCGCGTATCACCCAAGCTT (SEQ ID NO: 18)

Because HSV ICP4 promoters are subject to auto-repression by ICP4, the ICP4 binding site in the HSV-2 ICP4 promoter was deleted from the described tetO-containing HSV-2 ICP4/TO promoter.

Codon Optimized mmTGF-P2-7M with HSV-1 gD signal peptide at the N-terminal:
gccaccATGGGCGGCGCCGCCGCCCGCCTGGGCGCCGTGATCCTGTTCGTGGTGATC
GTGGGCCTGCACGGCGTGCGCGGCGCCCTGGACGCCGCCTACTGCTTCCGCAACGTGCAGGACAACTG
CTGCCTGCGCCCCCTGTACATCGACTTCCGCAAGGACCTGGGCTGGAAGTGGATCCACGAGCCCAAGG
GCTACAACGCCAACTTCTGCGCCGGCGCCTGCCCC
TACCGCGCCAGCAAGAGCCCCAGCTGCGTGAGCCAGGACCTGGAGCCCCT
GACCATCGTGTACTACGTGGGCCGCAAGCCCAAGGTGGAGCAGCTGAGCA
ACATGATCGTGAAGAGCTGCAAGTGCAGCTAAgaattc (SEQ ID NO: 19)
Codon Optimized gD signal peptide sequence:
ATGGGCGGCGCCGCCGCCCGCCTGGGCGCCGTGATCCTGTTCGTGGTGAT
CGTGGGCCTGCACGGCGTGCGCGGC(SEQ ID NO: 20)
Atezolizumab (USAN/INN); Atezolizumab (genetical recombination) (JAN); Tecentriq (TN)
Heavy Chain (448 Amino acids):
EVQLVESGGG LVQPGGSLRL SCAASGFTFS DSWIHWVRQA PGKGLEWVAW ISPYGGSTYY
ADSVKGRFTI SADTSKNTAY LQMNSLRAED TAVYYCARRH WPGGFDYWGQ GTLVTVSSAS
TKGPSVFPLA PSSKSTSGGT AALGCLVKDY FPEPVTVSWN SGALTSGVHT FPAVLQSSGL
YSLSSVVTVP SSSLGTQTYI CNVNHKPSNT KVDKKVEPKS CDKTHTCPPC PAPELLGGPS
VFLFPPKPKD TLMISRTPEV TCVVVDVSHE DPEVKFNWYV DGVEVHNAKT KPREEQYAST
YRVVSVLTVL HQDWLNGKEY KCKVSNKALP APIEKTISKA KGQPREPQVY TLPPSREEMT
KNQVSLTCLV KGFYPSDIAV EWESNGQPEN NYKTTPPVLD SDGSFFLYSK LTVDKSRWQQ
GNVFSCSVMH EALHNHYTQK SLSLSPGK (SEQ ID NO: 21)
Light Chain (214 Amino acids):
DIQMTQSPSS LSASVGDRVT ITCRASQDVS TAVAWYQQKP GKAPKLLIYS ASFLYSGVPS
RFSGSGSGTD FTLTISSLQP EDFATYYCQQ YLYHPATFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC (SEQ ID NO: 22)
Atezolizumab Heavy Chain consisting of mmTGF-beta 2-7M with GGGGGGS linker fused to
the C-terminal of the heavy chain:
EVQLVESGGG LVQPGGSLRL SCAASGFTFS DSWIHWVRQA PGKGLEWVAW
ISPYGGSTYY ADSVKGRFTI SADTSKNTAY LQMNSLRAED TAVYYCARRH WPGGFDYWGQ
GTLVTVSSAS TKGPSVFPLA PSSKSTSGGT AALGCLVKDY FPEPVTVSWN SGALTSGVHT
FPAVLQSSGL YSLSSVVTVP SSSLGTQTYI CNVNHKPSNT KVDKKVEPKS CDKTHTCPPC
PAPELLGGPS VFLFPPKPKD TLMISRTPEV TCVVVDVSHE DPEVKFNWYV DGVEVHNAKT
KPREEQYAST
YRVVSVLTVL HQDWLNGKEY KCKVSNKALP APIEKTISKA KGQPREPQVY TLPPSREEMT
KNQVSLTCLV KGFYPSDIAV EWESNGQPEN NYKTTPPVLD SDGSFFLYSK LTVDKSRWQQ
GNVFSCSVMH EALHNHYTQK SLSLSPGK
GGGGGGS ALDAAYCFRN VQDNCCLRPL YIDFRKDLGW KWIHEPKGYN ANFCAGACPY
RASKSPSCVS QDLEPLTIVY YVGRKPKVEQ LSNMIVKSCK CS (SEQ ID NO: 23)

The fusion protein could be no linker or with 2-4 copies of linker. The linker could also be GGGGS (SEQ ID NO: 27), or GGGGGS (SEQ ID NO: 28) or other linker commonly used for fusing 2 different functional proteins.

KEGG DRUG: (e.g., Pembrolizuma; Pembrolizumab (USAN); Pembrolizumab (genetical
recombination) (JAN); Keytruda (TN))
Heavy Chain (447 Amino acids)
QVQLVQSGVE VKKPGASVKV SCKASGYTFT NYYMYWVRQA PGQGLEWMGG INPSNGGTNF
NEKFKNRVTL TTDSSTTTAY MELKSLQFDD TAVYYCARRD YRFDMGFDYW GQGTTVTVSS
ASTKGPSVFP LAPCSRSTSE STAALGCLVK DYFPEPVTVS WNSGALTSGV HTFPAVLQSS
GLYSLSSVVT VPSSSLGTKT YTCNVDHKPS NTKVDKRVES KYGPPCPPCP APEFLGGPSV
FLFPPKPKDT LMISRTPEVT CVVVDVSQED PEVQFNWYVD GVEVHNAKTK PREEQFNSTY
RVVSVLTVLH QDWLNGKEYK CKVSNKGLPS SIEKTISKAK GQPREPQVYT LPPSQEEMTK
NQVSLTCLVK GFYPSDIAVE WESNGQPENN YKTTPPVLDS DGSFFLYSRL TVDKSRWQEG
NVFSCSVMHE ALHNHYTQKS LSLSLGK (SEQ ID NO: 24)
Light Chain (218 Amino acids)
EIVLTQSPAT LSLSPGERAT LSCRASKGVS TSGYSYLHWY QQKPGQAPRL LIYLASYLES
GVPARFSGSG SGTDFTLTIS SLEPEDFAVY YCQHSRDLPL TFGGGTKVEI KRTVAAPSVF
IFPPSDEQLK SGTASVVCLL NNFYPREAKV QWKVDNALQS GNSQESVTEQ DSKDSTYSLS
STLTLSKADY EKHKVYACEV THQGLSSPVT KSFNRGEC (SEQ ID NO: 25)
Pembrolizumab Heavy Chain consisting of mmTGF-beta 2-7M with GGGGGGS linker fused to
the C-terminal of the heavy chain:
QVQLVQSGVE VKKPGASVKV SCKASGYTFT NYYMYWVRQA PGQGLEWMGG
INPSNGGTNF NEKFKNRVTL TTDSSTTTAY MELKSLQFDD TAVYYCARRD YRFDMGFDYW
GQGTTVTVSS ASTKGPSVFP LAPCSRSTSE STAALGCLVK DYFPEPVTVS WNSGALTSGV
HTFPAVLQSS GLYSLSSVVT VPSSSLGTKT YTCNVDHKPS NTKVDKRVES KYGPPCPPCP
APEFLGGPSV FLFPPKPKDT LMISRTPEVT CVVVDVSQED PEVQFNWYVD GVEVHNAKTK
PREEQFNSTY
RVVSVLTVLH QDWLNGKEYK CKVSNKGLPS SIEKTISKAK GQPREPQVYT LPPSQEEMTK
NQVSLTCLVK GFYPSDIAVE WESNGQPENN YKTTPPVLDS DGSFFLYSRL TVDKSRWQEG
NVFSCSVMHE ALHNHYTQKS LSLSLGK
GGGGGGS ALDAAYCFRN VQDNCCLRPL YIDFRKDLGW KWIHEPKGYN ANFCAGACPY
RASKSPSCVS QDLEPLTIVY YVGRKPKVEQ LSNMIVKSCK CS (SEQ ID NO: 26)

The fusion protein could be no linker or with 2-4 copies of linker. The linker could also be GGGGS (SEQ ID NO: 27), or GGGGGS (SEQ ID NO: 28).

1. An oncolytic Herpes Simplex Virus (HSV) comprising recombinant DNA, wherein the recombinant DNA comprises:
g) a gene comprising a 5′ untranslated region and a HSV-1, or HSV-2, VP5 gene that is operably linked to an VP5 promoter comprising a TATA element;
h) a tetracycline operator sequence positioned between 6 and 24 nucleotides 3′ to said TATA element, wherein the VP5 gene lies 3′ to said tetracycline operator sequence;
i) a gene sequence encoding tetracycline repressor operably linked to an HSV immediate-early promoter, wherein the gene sequence is located at the ICP0 locus;
j) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant;
k) a gene sequence encoding a functional ICP34.5 protein; and
l) a gene sequence operably linked to a modified HSV promoter, wherein the gene is located in an intergenic region of UL26 and UL27 genes,
wherein said oncolytic HSV does not encode functional ICP0 and does not contain a ribozyme sequence located in said 5′ untranslated region of VP5.

2. An oncolytic Herpes Simplex Virus (HSV) comprising recombinant DNA, wherein the recombinant DNA comprises:
g) a gene comprising a 5′ untranslated region and a HSV-1, or HSV-2, VP5 gene that is operably linked to an VP5 promoter comprising a TATA element;
h) a tetracycline operator sequence positioned between 6 and 24 nucleotides 3′ to said TATA element, wherein the VP5 gene lies 3′ to said tetracycline operator sequence;
i) a gene sequence encoding tetracycline repressor operably linked to an HSV immediate-early promoter, wherein the gene sequence is located at the ICP0 locus;
j) a variant gene that increases syncytium formation as compared to wild type, wherein the HSV-1, or HSV-2, variant gene is selected from the group consisting of a glycoprotein K (gK) variant; a glycoprotein B (gB) variant; a UL24 variant; and UL20 gene variant;
k) a gene sequence encoding a functional ICP34.5 protein; and
l) a gene sequence operably linked to a modified HSV promoter, wherein the gene is located in an intergenic region of UL21 and UL22 genes,
wherein said oncolytic HSV does not encode functional ICP0 and does not contain a ribozyme sequence located in said 5′ untranslated region of VP5.

3. An oncolytic Herpes Simplex Virus (HSV) comprising recombinant DNA, wherein the recombinant DNA comprises:
g) a gene comprising a 5′ untranslated region and a HSV-1, or HSV-2, VP5 gene that is operably linked to an VP5 promoter comprising a TATA element;
h) a tetracycline operator sequence positioned between 6 and 24 nucleotides 3′ to said TATA element, wherein the VP5 gene lies 3′ to said tetracycline operator sequence;
i) a gene sequence encoding tetracycline repressor operably linked to an HSV immediate-early promoter, wherein the gene sequence is located at the ICP0 locus;
j) a variant gene that increases syncytium formation as compared to wild type, wherein the HSV-1, or HSV-2, variant gene is selected from the group consisting of: a glycoprotein K (gK) variant; a glycoprotein B (gB) variant; a UL24 variant; and UL20 gene variant;
k) a gene sequence encoding a functional ICP34.5 protein; and
l) a gene sequence operably linked to a modified HSV promoter, wherein the gene is located in an intergenic region of UL26, UL27, UL21 and UL 21 genes,
wherein said oncolytic HSV does not encode functional ICP0 and does not contain a ribozyme sequence located in said 5′ untranslated region of VP5.

4. The oncolytic HSV of any of claims 1-3, wherein the gene sequence of (f) is a LacZ gene sequence.

5. The oncolytic HSV of any of claims 1-3, wherein the gene sequence of (f) is a dominant-negative TGF-β mutant sequence.

6. The oncolytic HSV of claim 5, wherein the dominant-negative TGF-β mutant sequence is a mmTGF-β2-7 M fragment sequence.

7. The oncolytic HSV of any of claims 1-3, wherein the promoter of (f) is a modified HSV immediate-early promoter, an HCMV immediate-early promoter, or a human elongation alpha promoter.

8. The oncolytic HSV of any of claims 1-3, wherein the variant gene is a gK variant gene that encodes an amino acid substitution selected from the group consisting of: an Ala to Thr amino acid substitution corresponding to amino acid 40 of SEQ ID NO: 2; an Ala to “x” amino acid substitution corresponding to amino acid 40 of SEQ ID NO: 2, wherein “x” is any amino acid; an Asp to Asn amino acid substitution corresponding to amino acid 99 of SEQ ID NO: 2; a Leu to Pro amino acid substitution corresponding to amino acid 304 of SEQ ID NO: 2; and an Arg to Leu amino acid substitution corresponding to amino acid 310 of SEQ ID NO: 2.

9. The oncolytic HSV of any of claims 1-8, wherein the tetracycline operator sequence comprises two Op2 repressor binding sites.

10. The oncolytic HSV of any of claims 1-9, wherein the VP5 promoter is an HSV-1 or HSV-2 VP5 promoter.

11. The oncolytic HSV of any of claims 1-10, wherein the immediate-early promoter is an HSV-1 or HSV-2 immediate-early promoter.

12. The oncolytic HSV of any of claims 1-11, wherein the HSV immediate-early promoter is selected from the group consisting of: ICP0 promoter, ICP4 promoter, and ICP27 promoter.

13. The oncolytic HSV of any of claims 1-12, wherein the recombinant DNA is part of the HSV-1 genome.

14. The oncolytic HSV of any of claims 1-12, wherein the recombinant DNA is part of the HSV-2 genome.

15. The oncolytic HSV of any of claims 1-13, further comprising a pharmaceutically acceptable carrier.

16. The oncolytic HSV of any of claims 1-15, further encoding at least one polypeptide that can increase the efficacy of the oncolytic HSV to induce an anti-tumor-specific immunity.

17. The oncolytic HSV of claim 16, wherein the at least one polypeptide encodes a product selected from the group consisting of: interleukin 2 (IL2), interleukin 12 (IL12), interleukin 15 (IL15), an anti-PD-1 antibody or antibody reagent, an anti-PD-L1 antibody or antibody reagent, an anti-OX40 antibody or antibody reagent, a CTLA-4 antibody or antibody reagent, a TIM-3 antibody or antibody reagent, a TIGIT antibody or antibody reagent, a soluble interleukin 10 receptor (IL10R), a fusion polypeptide between a soluble IL10R and IgG-Fc domain, a soluble TGFβ type II receptor (TGFBRII), a fusion polypeptide between a soluble TGFBRII and IgG-Fc domain, an anti-IL10R antibody or antibody reagent, an anti-IL10 antibody or antibody reagent, an anti-TGFBRII antibody or antibody reagent, and an anti-TGFBRII antibody or antibody reagent.

18. The oncolytic HSV of any of claims 1-17, wherein the oncolytic HSV the further encodes fusogenic activity.

19. An oncolytic Herpes Simplex Virus (HSV) comprising recombinant DNA, wherein the recombinant DNA comprises:
g) a gene comprising a 5′ untranslated region and a HSV-1, or HSV-2, VP5 gene that is operably linked to an VP5 promoter comprising a TATA element;
h) a tetracycline operator sequence positioned between 6 and 24 nucleotides 3′ to said TATA element, wherein the VP5 gene lies 3′ to said tetracycline operator sequence;
i) a gene sequence encoding tetracycline repressor operably linked to an HSV immediate-early promoter, wherein the gene sequence is located at the ICP0 locus;
j) a variant gene that increases syncytium formation as compared to wild type, wherein the HSV-1, or HSV-2, variant gene is selected from the group consisting of a glycoprotein K (gK) variant; a glycoprotein B (gB) variant; a UL24 variant; and UL20 gene variant;
k) a gene sequence encoding a functional ICP34.5 protein; and
l) a dominant-negative TGF-β mutant sequence operably linked to a modified HSV-2 immediate-early promoter, wherein the gene is located in an intergenic region of UL26 and UL27 genes.
wherein said oncolytic HSV does not encode functional ICP0 and does not contain a ribozyme sequence located in said 5′ untranslated region of VP5.

20. An oncolytic Herpes Simplex Virus (HSV) comprising recombinant DNA, wherein the recombinant DNA comprises:
g) a gene comprising a 5′ untranslated region and a HSV-1, or HSV-2, VP5 gene that is operably linked to an VP5 promoter comprising a TATA element;
h) a tetracycline operator sequence positioned between 6 and 24 nucleotides 3′ to said TATA element, wherein the VP5 gene lies 3′ to said tetracycline operator sequence;
i) a gene sequence encoding tetracycline repressor operably linked to an HSV immediate-early promoter, wherein the gene sequence is located at the ICP0 locus;
j) a variant gene that increases syncytium formation as compared to wild type, wherein the HSV-1, or HSV-2, variant gene is selected from the group consisting of a glycoprotein K (gK) variant; a glycoprotein B (gB) variant; a UL24 variant; and UL20 gene variant;
k) a gene sequence encoding a functional ICP34.5 protein; and
l) a dominant-negative TGF-β mutant sequence operably linked to a modified HSV-2 immediate-early promoter, wherein the gene is located in an intergenic region of UL21 and UL22 genes.
wherein said oncolytic HSV does not encode functional ICP0 and does not contain a ribozyme sequence located in said 5′ untranslated region of VP5.

21. An oncolytic Herpes Simplex Virus (HSV) comprising recombinant DNA, wherein the recombinant DNA comprises:
g) a gene comprising a 5′ untranslated region and a HSV-1, or HSV-2, VP5 gene that is operably linked to an VP5 promoter comprising a TATA element;
h) a tetracycline operator sequence positioned between 6 and 24 nucleotides 3′ to said TATA element, wherein the VP5 gene lies 3′ to said tetracycline operator sequence;
i) a gene sequence encoding tetracycline repressor operably linked to an HSV immediate-early promoter, wherein the gene sequence is located at the ICP0 locus;
j) a variant gene that increases syncytium formation as compared to wild type, wherein the HSV-1, or HSV-2, variant gene is selected from the group consisting of a glycoprotein K (gK) variant; a glycoprotein B (gB) variant; a UL24 variant; and UL20 gene variant;
k) a gene sequence encoding a functional ICP34.5 protein; and
l) a dominant-negative TGF-β mutant sequence operably linked to a modified HSV-2 immediate-early promoter, wherein the gene is located in an intergenic region of UL 21, UL22, UL26 and UL27 genes.
wherein said oncolytic HSV does not encode functional ICP0 and does not contain a ribozyme sequence located in said 5′ untranslated region of VP5.

22. The oncolytic HSV of any of claims 19-21, wherein the dominant-negative TGF-β mutant sequence is a mmTGF-β2-7 M fragment sequence.

23. The oncolytic HSV of any of claims 19-21, wherein the HSV-2 immediate-early promoter of (f) is selected from the group consisting of ICP0, ICP4, and ICP27.

24. The oncolytic HSV of any of claims 19-21, wherein the HSV-2 immediate-early promoter of (f) is tet operator-containing.

25. The oncolytic HSV of any of claims 19-21, wherein the HSV is tetracycline or doxycycline-regulatable.

26. An oncolytic Herpes Simplex Virus (HSV) comprising recombinant DNA, wherein the recombinant DNA does not encode functional ICP0 or ICP34.5 genes; and encodes a functional mmTGF-β2-7 M fragment sequence.

27. An oncolytic Herpes Simplex Virus (HSV) comprising recombinant DNA, wherein the recombinant DNA does not encode functional ICP0; and encodes a functional mmTGF-β2-7 M fragment sequence.

28. The oncolytic HSV of claim 26 or 27, wherein the HSV further encodes fusogenic activity.

29. The oncolytic HSV of claim 26 or 27, wherein the HSV is tetracycline or doxycycline-regulatable.

30. An oncolytic virus encoding a functional mmTGF-β2-7 M fragment sequence.

31. A recombinant virus encoding a functional mmTGF-β2-7 M fragment sequence.

32. A composition comprising a virus of any of claims 1-31.

33. The composition of claim 32, further comprising a pharmaceutically acceptable carrier.

34. A cell expressing any of the viruses of any of claims 1-31 or composition of claims 32-33.

35. The cell of claim 34, wherein the cell is mammalian.

36. The cell of claims 34-35, wherein the cell is a cancer cell or an immune cell.

37. The cell of claim 36, wherein the immune cell is a B cell or T cell.

38. The cell of any of claims 34-37, wherein the cell expresses high levels of mmTGF-β2-7 M.

39. A method for treating cancer, the method comprising administering the virus of any of claims 1-31 or the composition of any of claims 32-33 to a subject having cancer.

40. The method of claim 39, wherein the cancer is a solid tumor.

41. The method of claim 40, wherein the tumor is benign or malignant.

42. The method of any of claims 39-41, wherein the subject is diagnosed or has been diagnosed as having cancer is selected from the list consisting of a carcinoma, a melanoma, a sarcoma, a germ cell tumor, and a blastoma.

43. The method of any of claims 39-42, wherein the subject is diagnosed or has been diagnosed as having a cancer selected from the group consisting of non-small-cell lung cancer, bladder cancer, breast cancer, brain cancer, colon cancer, prostate cancer, liver cancer, lung cancer, ovarian cancer, skin cancer, head and neck cancer, kidney cancer, and pancreatic cancer.

44. The method of any of claims 39-43, wherein the cancer is metastatic.

45. The method of any of claims 39-44, further comprising administering an agent that regulates the tet operator-containing promoter.

46. The method of claim 45, wherein the agent is doxycycline or tetracycline.

47. The method of claim 46, wherein the agent is administered locally or systemically.

48. The method of claim 47, wherein the systemic administration is oral administration.

49. The method of any of claims 39-48, wherein the virus or composition is administered directly to the tumor.

50. A hybrid nucleic acid sequence containing a sequence of a therapeutic antibody and mmTGF-β2-7 M, wherein mmTGF-β2-7 M is fused to a Fc domain of the therapeutic antibody.

51. The hybrid nucleic acid sequence of claim 50, wherein the therapeutic antibody sequence is a sequence of an immunotherapeutic antibody.

52. The hybrid nucleic acid sequence of claim 50 or 51, wherein the therapeutic antibody sequence is a sequence selected from the list consisting of an anti-PD-1 antibody, an anti-PD-L1 antibody, an anti-Tim3 antibody, an anti-anti-CTLA4 antibody, and anti-TDM-1 antibody, and an anti-TIGIT antibody.

53. A polypeptide encoded by the hybrid nucleic acid of any of claims 50-52.

54. A vector expressing any of the hybrid nucleic acid of any of claims 50-52 or polypeptides of claim 53.

55. A chimeric antigen receptor (CAR) polypeptide comprising at least one of
a. an extracellular domain comprising a dominant-negative TGF-β mutant sequence;
b. a transmembrane domain;
c. a co-stimulatory domain; and
d. an intracellular signaling domain.

56. The CAR polypeptide of claim 55, wherein the dominant-negative TGF-β mutant sequence is a mmTGF-β2-7 M fragment sequence.

57. A nucleic acid encoding the CAR polypeptide of claims 55 or 56.

58. A mammalian cell comprising:
a. the CAR polypeptide of claims 55 or 56; or
b. a nucleic acid encoding of claim 57.

59. The cell of claim 58, wherein the cell is a T cell.

60. The cell of claims 58 or 59, wherein the cell is a human cell.

61. The cell of any of claims 58-60, further comprising at least a second CAR polypeptide.

62. The cell of claim 61, where the at least second CAR polypeptide comprises an extracellular domain comprising a sequence that binds a checkpoint inhibitor.

63. The cell of claim 62, where the checkpoint inhibitor is selected from the group selected from: PD-L1, PD-1, TIGIT, TIM3, and CTLA4.

64. The cell of any of claims 58-63, wherein the cell is obtained from an individual having or diagnosed as having cancer.

65. A method of treating cancer in a subject in need thereof, the method comprising administering the cell of any of claims 58-64 to the subject.

66. A method of treating cancer in a subject in need thereof, the method comprising:
a. engineering a T cell to comprise the CAR polypeptide of claims 55 or 56 or nucleic acid encoding of claim 57 on the T cell surface; and
b. administering the engineered T cell to the subject.

67. The method of claim 66, wherein the engineered T cell further comprises at least a second CAR polypeptide.

68. The method of any of the previous claims, further comprising administering at least one additional anti-cancer therapeutic.

69. An oncolytic Herpes Simplex Virus (HSV) comprising recombinant DNA, wherein the recombinant DNA does not encode functional ICP0 and ICP34.5 genes; and encodes a functional mmTGF-β2-7 M fragment sequence.


SEQUENCE LISTING (30 sequences)

SEQ ID NO: 1 - skipped

SEQ ID NO: 2 - protein, 338 aa, Artificial Sequence
  Description of Artificial Sequence: Synthetic polypeptide
MLAVRSLQHLSTVVLITAYGLVLVWYTVFGASPLHRCIYAVRPTGTNNDTALVWMKMNQTLLFLGAPTHPPNGGWRNHAHICYANLIAGRVVPFQVPPDATNRRIMNVHEAVNCLETLWYTRVRLVVVGWFLYLAFVALHQRRCMFGVVSPAHKMVAPATYLLNYAGRIVSSVFLQYPYTKITRLLCELSVQRQNLVQLFETDPVTFLYHRPAIGVIVGCELMLRFVAVGLIVGTAFISRGACAITYPLFLTITTWCFVSTIGLTELYCILRRGPAPKNADKAAAPGRSKGLSGVCGRCCSIILSGIAMRLCYIAVVAGVVLVALHYEQEIQRRLFDV

SEQ ID NO: 3 - DNA, 1737 bp, Unknown
  Description of Unknown: IL10R sequence
atgctgccgtgcctcgtagtgctgctggcggcgctcctcagcctccgtcttggctcagacgctcatgggacagagctgcccagccctccgtctgtgtggtttgaagcagaatttttccaccacatcctccactggacacccatcccaaatcagtctgaaagtacctgctatgaagtggcgctcctgaggtatggaatagagtcctggaactccatctccaactgtagccagaccctgtcctatgaccttaccgcagtgaccttggacctgtaccacagcaatggctaccgggccagagtgcgggctgtggacggcagccggcactccaactggaccgtcaccaacacccgcttctctgtggatgaagtgactctgacagttggcagtgtgaacctagagatccacaatggcttcatcctcgggaagattcagctacccaggcccaagatggcccccgcaaatgacacatatgaaagcatcttcagtcacttccgagagtatgagattgccattcgcaaggtgccgggaaacttcacgttcacacacaagaaagtaaaacatgaaaacttcagcctcctaacctctggagaagtgggagagttctgtgtccaggtgaaaccatctgtcgcttcccgaagtaacaaggggatgtggtctaaagaggagtgcatctccctcaccaggcagtatttcaccgtgaccaacgtcatcatcttctttgcctttgtcctgctgctctccggagccctcgcctactgcctggccctccagctgtatgtgcggcgccgaaagaagctacccagtgtcctgctcttcaagaagcccagccccttcatcttcatcagccagcgtccctccccagagacccaagacaccatccacccgcttgatgaggaggcctttttgaaggtgtccccagagctgaagaacttggacctgcacggcagcacagacagtggctttggcagcaccaagccatccctgcagactgaagagccccagttcctcctccctgaccctcacccccaggctgacagaacgctgggaaacagggagccccctgtgctgggggacagctgcagtagtggcagcagcaatagcacagacagcgggatctgcctgcaggagcccagcctgagccccagcacagggcccacctgggagcaacaggtggggagcaacagcaggggccaggatgacagtggcattgacttagttcaaaactctgagggccgggctggggacacacagggtggctcggccttgggccaccacagtcccccggagcctgaggtgcctggggaagaagacccagctgctgtggcattccagggttacctgaggcagaccagatgtgctgaagagaaggcaaccaagacaggctgcctggaggaagaatcgcccttgacagatggccttggccccaaattcgggagatgcctggttgatgaggcaggcttgcatccaccagccctggccaagggctatttgaaacaggatcctctagaaatgactctggcttcctcaggggccccaacgggacagtggaaccagcccactgaggaatggtcactcctggccttgagcagctgcagtgacctgggaatatctgactggagctttgcccatgaccttgcccctctaggctgtgtggcagccccaggtggtctcctgggcagctttaactcagacctggtcaccctgcccctcatctctagcctgcagtcaagtgagtga

SEQ ID NO: 4 - DNA, 1788 bp, Unknown
  Description of Unknown: TGFBRII sequence
atgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgttgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaatagctcttctgg

SEQ ID NO: 5 - DNA, 465 bp, Unknown
  Description of Unknown: IL-2 sequence
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataa

SEQ ID NO: 6 - DNA, 763 bp, Unknown
  Description of Unknown: IL-12a sequence
aatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 7 - DNA, 489 bp, Unknown
  Description of Unknown: IL-15 sequence
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 8 - DNA, 19 bp, Artificial Sequence
  Description of Artificial Sequence: Synthetic oligonucleotide
tccctatcagtgatagaga

SEQ ID NO: 9 - skipped

SEQ ID NO: 10 - DNA, 282 bp, Artificial Sequence
  Description of Artificial Sequence: Synthetic polynucleotide
atggccctggacgccgcctactgcttccgcaacgtgcaggacaactgctgcctgcgccccctgtacatcgacttccgcaaggacctgggctggaagtggatccacgagcccaagggctacaacgccaacttctgcgccggcgcctgcccctaccgcgccagcaagagccccagctgcgtgagccaggacctggagcccctgaccatcgtgtactacgtgggccgcaagcccaaggtggagcagctgagcaacatgatcgtgaagagctgcaagtgcagctaa

SEQ ID NO: 11 - protein, 92 aa, Artificial Sequence
  Description of Artificial Sequence: Synthetic polypeptide
ALDAAYCFRNVQDNCCLRPLYIDFRKDLGWKWIHEPKGYNANFCAGACPYRASKSPSCVSQDLEPLTIVYYVGRKPKVEQLSNMIVKSCKCS

SEQ ID NO: 12 - DNA, 2730 bp, Artificial Sequence
  Description of Artificial Sequence: Synthetic polynucleotide
tcccattacaaccagctcgtcgccggccacgccgcgccccaaccccagccgcattccgcgtttggtttcccggctgcggcgggggccgtggcctatgggcctcacggcgcgggtctttcccagcattaccctccccacgtcgcccatcagtatcccggggtgctgttctcgggacccagcccactcgaggcgcagatagccgcgttggtgggggccatagccgcggaccgccaggcgggcggtcagccggccgcgggagaccctggggtccgggggtcgggaaagcgtcgccggtacgaggcggggccgtcggagtcctactgcgaccaggacgaaccggacgcggactacccgtactaccccggggaggctcgaggcgggccgcgcggggtcgactctcggcgcgcggcccgccagtctcccgggaccaacgagaccatcacggcgctgatgggggcggtgacgtctctgcagcaggaactggcgcacatgcgggctcggaccagcgccccctatggaatgtacacgccggtggcgcactatcgccctcaggtgggggagccggaaccaacaacgacccacccggccctttgtcccccggaggccgtgtatcgccccccaccacacagcgccccctacggtcctccccagggtccggcgtcccatgcccccactcccccgtatgccccagctgcctgcccgccaggcccgccaccgcccccatgtccttccacccagacgcgcgcccctctaccgacggagcccgcgttcccccccgccgccaccggatcccaaccggaggcatccaacgcggaggccggggcccttgtcaacgccagcagcgcagcacacgtggacgttgacacggcccgcgccgccgatttgttcgtctctcagatgatgggggcccgctgattcgccccggtctttggtaccatgggatgtcttactgtatatctttttaaataaaccaggtaataccaaataagacccattggtgtatgttctttttttattggcgcgccatatgcattcccgcttatgctaatgagatgcgttcggacggcccctcgctccccgcgtaattactccctcggggttccgggttatgctgattactttcttggcagaacacgcagagcctcgcgcgccgccgggtgggtgggctgatcggcccctattggtcccctgggcttcctagtatgctaatgatattctccccgggggcgggcaccactcagggccgcgccggcggggcgccggggggactcccatctgcgtcggcggggggcggcgcatgctaatgatattcttggagtacacccggttggtccccggggacggggccgccccgagagggggggattccctccctccgcccccgccggggcgcgcggctattgggggaatcgtaaatgccgcccctttgggggagtggataggcgccgggtatataagcagagctctccctatcagtgatagagatctccctatcagtgatagagatcgtcgacgagctcgcgaagcttaccggtcgatatctatcgcgagctcgccaattgcgagatctagaagcggaattctgcccgtcagaacttgtttattgcagcttataatggttacaaataaagcaatagcatcacaaatttcacaaataaagcatttttttcactgcattctagttgtggtttgtccaaactcatcaatgtatcttatcatgtctggttacccaaaaggcgtaccaaacaaaaaaaccaaaagatgcgcatgcggtttaacacccgtggtttttatttacaacaaaccccccgtcacaggtcgtcctcgtcggcgtcaccgtctttgttgggaacttgggtgtagttggtgttgcggcgcttgcgcatgaccatgtcggtgaccttggcgctgagcagcgcgctcgtgcccttcttcttggccttgtgttccgtgcgctccatggccgacaccagggccatgtaccgtatcatctccctggcctcggctagcttggcctcgtcaaagtcgccgccctcctcgccctccccggacgcgtccgggttggtggggttcttgagctccttggtggttagagggtacagggccttcatggggttgctctgcagccgcatgacgtaacgaaaggcgaagaaggccgccgccaggccggccaggaccaacagacccacggccagcgccccaaaggggttggacatgaaggaggacacgcccgacacggccgataccacgccgcccacgatgcccatcaccaccttgccgaccgcgcgccccaggtcgcccatcccctcgaagaacgcgcccaggcccgcgaacatggcggcgttggcgtcggcgtggatgaccgtgtcgatgtcggcgaagcgcaggtcgtgcagctggttgcggcgctggacctccgtgtagtccagcaggccgctgtccttgatctcgtggcgggtgtacacctccagggggacaaactcgtgatcctccagcatggtgatgttgaggtcgatgaaggtgctgacggtggtgatgtcggcgcggctcagctggtgggagtacgcgtactcctcgaagtacacgtagcccccaccgaaggtgaagtagcgccggtgtcccacggtgcacggctcgatcgcatcgcgcgtcagccgcagctcgttgttctcccccagctgcccctcgaccaacgggccctggtcttcgtaccgaaagctgaccaggggg

SEQ ID NO: 13 - DNA, 1000 bp, Artificial Sequence
  Description of Artificial Sequence: Synthetic polynucleotide
tcccattacaaccagctcgtcgccggccacgccgcgccccaaccccagccgcattccgcgtttggtttcccggctgcggcgggggccgtggcctatgggcctcacggcgcgggtctttcccagcattaccctccccacgtcgcccatcagtatcccggggtgctgttctcgggacccagcccactcgaggcgcagatagccgcgttggtgggggccatagccgcggaccgccaggcgggcggtcagccggccgcgggagaccctggggtccgggggtcgggaaagcgtcgccggtacgaggcggggccgtcggagtcctactgcgaccaggacgaaccggacgcggactacccgtactaccccggggaggctcgaggcgggccgcgcggggtcgactctcggcgcgcggcccgccagtctcccgggaccaacgagaccatcacggcgctgatgggggcggtgacgtctctgcagcaggaactggcgcacatgcgggctcggaccagcgccccctatggaatgtacacgccggtggcgcactatcgccctcaggtgggggagccggaaccaacaacgacccacccggccctttgtcccccggaggccgtgtatcgccccccaccacacagcgccccctacggtcctccccagggtccggcgtcccatgcccccactcccccgtatgccccagctgcctgcccgccaggcccgccaccgcccccatgtccttccacccagacgcgcgcccctctaccgacggagcccgcgttcccccccgccgccaccggatcccaaccggaggcatccaacgcggaggccggggcccttgtcaacgccagcagcgcagcacacgtggacgttgacacggcccgcgccgccgatttgttcgtctctcagatgatgggggcccgctgattcgccccggtctttggtaccatgggatgtcttactgtatatctttttaaataaaccaggtaataccaaataagacccattggtg

SEQ ID NO: 14 - DNA, 1000 bp, Artificial Sequence
  Description of Artificial Sequence: Synthetic polynucleotide
caaaaggcgtaccaaacaaaaaaaccaaaagatgcgcatgcggtttaacacccgtggtttttatttacaacaaaccccccgtcacaggtcgtcctcgtcggcgtcaccgtctttgttgggaacttgggtgtagttggtgttgcggcgcttgcgcatgaccatgtcggtgaccttggcgctgagcagcgcgctcgtgcccttcttcttggccttgtgttccgtgcgctccatggccgacaccagggccatgtaccgtatcatctccctggcctcggctagcttggcctcgtcaaagtcgccgccctcctcgccctccccggacgcgtccgggttggtggggttcttgagctccttggtggttagagggtacagggccttcatggggttgctctgcagccgcatgacgtaacgaaaggcgaagaaggccgccgccaggccggccaggaccaacagacccacggccagcgccccaaaggggttggacatgaaggaggacacgcccgacacggccgataccacgccgcccacgatgcccatcaccaccttgccgaccgcgcgccccaggtcgcccatcccctcgaagaacgcgcccaggcccgcgaacatggcggcgttggcgtcggcgtggatgaccgtgtcgatgtcggcgaagcgcaggtcgtgcagctggttgcggcgctggacctccgtgtagtccagcaggccgctgtccttgatctcgtggcgggtgtacacctccagggggacaaactcgtgatcctccagcatggtgatgttgaggtcgatgaaggtgctgacggtggtgatgtcggcgcggctcagctggtgggagtacgcgtactcctcgaagtacacgtagcccccaccgaaggtgaagtagcgccggtgtcccacggtgcacggctcgatcgcatcgcgcgtcagccgcagctcgttgttctcccccagctgcccctcgaccaacgggccctggtcttcgtaccgaaagctgaccaggggg

SEQ ID NO: 15 - DNA, 694 bp, Artificial Sequence
  Description of Artificial Sequence: Synthetic polynucleotide
cattcccgcttatgctaatgagatgcgttcggacggcccctcgctccccgcgtaattactccctcggggttccgggttatgctgattactttcttggcagaacacgcagagcctcgcgcgccgccgggtgggtgggctgatcggcccctattggtcccctgggcttcctagtatgctaatgatattctccccgggggcgggcaccactcagggccgcgccggcggggcgccggggggactcccatctgcgtcggcggggggcggcgcatgctaatgatattcttggagtacacccggttggtccccggggacggggccgccccgagagggggggattccctccctccgcccccgccggggcgcgcggctattgggggaatcgtaaatgccgcccctttgggggagtggataggcgccgggtatataagcagagctctccctatcagtgatagagatctccctatcagtgatagagatcgtcgacgagctcgcgaagcttaccggtcgatatctatcgcgagctcgccaattgcgagatctagaagcggaattctgcccgtcagaacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 16 - DNA, 134 bp, Artificial Sequence
  Description of Artificial Sequence: Synthetic polynucleotide
cagaacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 17 - DNA, 806 bp, Artificial Sequence
  Description of Artificial Sequence: Synthetic polynucleotide
ggcgcgccgggccggcgggggccaacgggagcgcggggccggcatctcattaccacgaacccggaagggcaggggagcgagcccgcccgcgacgagggtctcattagcatcgcgggcggaagcggaagccgcccgcgccgggcgctaatgagatgccgcgcgggcggagcggcggcggcgcgaccaacgggccgccgccacggacgcggacgcgcgggcgtcggggcggggccgcgcataatgcggttccacctgggggcggaaccccggcgagccggggcgcggcggcgtcgatcgctcctcctccgcgtcctcctcctttccccccgccccgcgcgccccgaggactatatgagccgagctctccctatcagtgatagagatctccctatcagtgatagagatcgagctcgcgtgtgcatcgcgtatcacccaagcttgccaccatgggcggcgccgccgcccgcctgggcgccgtgatcctgttcgtggtgatcgtgggcctgcacggcgtgcgcggcgccctggacgccgcctactgcttccgcaacgtgcaggacaactgctgcctgcgccccctgtacatcgacttccgcaaggacctgggctggaagtggatccacgagcccaagggctacaacgccaacttctgcgccggcgcctgcccctaccgcgccagcaagagccccagctgcgtgagccaggacctggagcccctgaccatcgtgtactacgtgggccgcaagcccaaggtggagcagctgagcaacatgatcgtgaagagctgcaagtgcagctaagaattc

SEQ ID NO: 18 - DNA, 440 bp, Artificial Sequence
  Description of Artificial Sequence: Synthetic polynucleotide
ggcgcgccgggccggcgggggccaacgggagcgcggggccggcatctcattaccacgaacccggaagggcaggggagcgagcccgcccgcgacgagggtctcattagcatcgcgggcggaagcggaagccgcccgcgccgggcgctaatgagatgccgcgcgggcggagcggcggcggcgcgaccaacgggccgccgccacggacgcggacgcgcgggcgtcggggcggggccgcgcataatgcggttccacctgggggcggaaccccggcgagccggggcgcggcggcgtcgatcgctcctcctccgcgtcctcctcctttccccccgccccgcgcgccccgaggactatatgagccgagctctccctatcagtgatagagatctccctatcagtgatagagatcgagctcgcgtgtgcatcgcgtatcacccaagctt

SEQ ID NO: 19 - DNA, 366 bp, Artificial Sequence
  Description of Artificial Sequence: Synthetic polynucleotide
gccaccatgggcggcgccgccgcccgcctgggcgccgtgatcctgttcgtggtgatcgtgggcctgcacggcgtgcgcggcgccctggacgccgcctactgcttccgcaacgtgcaggacaactgctgcctgcgccccctgtacatcgacttccgcaaggacctgggctggaagtggatccacgagcccaagggctacaacgccaacttctgcgccggcgcctgcccctaccgcgccagcaagagccccagctgcgtgagccaggacctggagcccctgaccatcgtgtactacgtgggccgcaagcccaaggtggagcagctgagcaacatgatcgtgaagagctgcaagtgcagctaagaattc

SEQ ID NO: 20 - DNA, 75 bp, Artificial Sequence
  Description of Artificial Sequence: Synthetic oligonucleotide
atgggcggcgccgccgcccgcctgggcgccgtgatcctgttcgtggtgatcgtgggcctgcacggcgtgcgcggc

SEQ ID NO: 21 - protein, 448 aa, Artificial Sequence
  Description of Artificial Sequence: Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 214 aa, Artificial Sequence
  Description of Artificial Sequence: Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 547 aa, Artificial Sequence
  Description of Artificial Sequence: Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGGSALDAAYCFRNVQDNCCLRPLYIDFRKDLGWKWIHEPKGYNANFCAGACPYRASKSPSCVSQDLEPLTIVYYVGRKPKVEQLSNMIVKSCKCS

SEQ ID NO: 24 - protein, 447 aa, Artificial Sequence
  Description of Artificial Sequence: Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 218 aa, Artificial Sequence
  Description of Artificial Sequence: Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 546 aa, Artificial Sequence
  Description of Artificial Sequence: Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGGGSALDAAYCFRNVQDNCCLRPLYIDFRKDLGWKWIHEPKGYNANFCAGACPYRASKSPSCVSQDLEPLTIVYYVGRKPKVEQLSNMIVKSCKCS

SEQ ID NO: 27 - protein, 5 aa, Artificial Sequence
  Description of Artificial Sequence: Synthetic peptide
GGGGS

SEQ ID NO: 28 - protein, 6 aa, Artificial Sequence
  Description of Artificial Sequence: Synthetic peptide
GGGGGS

SEQ ID NO: 29 - protein, 4 aa, Artificial Sequence
  Description of Artificial Sequence: Synthetic peptide
RGDS

SEQ ID NO: 30 - protein, 7 aa, Artificial Sequence
  Description of Artificial Sequence: Synthetic peptide
GGGGGGS